SEQUENCE LISTING (6 sequences)

SEQ ID NO: 1 - RNA, 3413 nt, unidentified
  modified_base 2: mod_base = OTHER; note = thymine
  modified_base 7: mod_base = OTHER; note = thymine
  modified_base 15: mod_base = OTHER; note = thymine
  modified_base 18: mod_base = OTHER; note = thymine
  modified_base 25..28: mod_base = OTHER; note = thymine
  modified_base 31..32: mod_base = OTHER; note = thymine
  modified_base 37: mod_base = OTHER; note = thymine
  modified_base 39: mod_base = OTHER; note = thymine
  modified_base 42..43: mod_base = OTHER; note = thymine
  modified_base 45: mod_base = OTHER; note = thymine
  modified_base 49: mod_base = OTHER; note = thymine
  modified_base 51..54: mod_base = OTHER; note = thymine
  modified_base 56: mod_base = OTHER; note = thymine
  modified_base 58..60: mod_base = OTHER; note = thymine
  modified_base 69: mod_base = OTHER; note = thymine
  modified_base 78: mod_base = OTHER; note = thymine
  modified_base 82..83: mod_base = OTHER; note = thymine
  modified_base 90: mod_base = OTHER; note = thymine
  modified_base 98..99: mod_base = OTHER; note = thymine
  modified_base 102..103: mod_base = OTHER; note = thymine
  modified_base 107..108: mod_base = OTHER; note = thymine
  modified_base 111: mod_base = OTHER; note = thymine
  modified_base 120: mod_base = OTHER; note = thymine
  modified_base 128..132: mod_base = OTHER; note = thymine
  modified_base 142: mod_base = OTHER; note = thymine
  modified_base 144: mod_base = OTHER; note = thymine
  modified_base 150..151: mod_base = OTHER; note = thymine
  modified_base 154: mod_base = OTHER; note = thymine
  modified_base 156..157: mod_base = OTHER; note = thymine
  modified_base 159..160: mod_base = OTHER; note = thymine
  modified_base 165: mod_base = OTHER; note = thymine
  modified_base 169: mod_base = OTHER; note = thymine
  modified_base 171: mod_base = OTHER; note = thymine
  modified_base 174..175: mod_base = OTHER; note = thymine
  modified_base 180: mod_base = OTHER; note = thymine
  modified_base 183..184: mod_base = OTHER; note = thymine
  modified_base 190: mod_base = OTHER; note = thymine
  modified_base 192: mod_base = OTHER; note = thymine
  modified_base 195: mod_base = OTHER; note = thymine
  modified_base 200..201: mod_base = OTHER; note = thymine
  modified_base 203: mod_base = OTHER; note = thymine
  modified_base 211: mod_base = OTHER; note = thymine
  modified_base 214..216: mod_base = OTHER; note = thymine
  modified_base 218..219: mod_base = OTHER; note = thymine
  modified_base 224..225: mod_base = OTHER; note = thymine
  modified_base 227..229: mod_base = OTHER; note = thymine
  modified_base 231: mod_base = OTHER; note = thymine
  modified_base 233..234: mod_base = OTHER; note = thymine
  modified_base 237: mod_base = OTHER; note = thymine
  modified_base 243..244: mod_base = OTHER; note = thymine
  modified_base 254: mod_base = OTHER; note = thymine
  modified_base 257: mod_base = OTHER; note = thymine
  modified_base 271..273: mod_base = OTHER; note = thymine
  modified_base 279: mod_base = OTHER; note = thymine
  modified_base 285: mod_base = OTHER; note = thymine
  modified_base 292: mod_base = OTHER; note = thymine
  modified_base 296..297: mod_base = OTHER; note = thymine
  modified_base 304: mod_base = OTHER; note = thymine
  modified_base 306: mod_base = OTHER; note = thymine
  modified_base 308: mod_base = OTHER; note = thymine
  modified_base 311..312: mod_base = OTHER; note = thymine
  modified_base 314: mod_base = OTHER; note = thymine
  modified_base 318: mod_base = OTHER; note = thymine
  modified_base 321: mod_base = OTHER; note = thymine
  modified_base 325: mod_base = OTHER; note = thymine
  modified_base 327: mod_base = OTHER; note = thymine
  modified_base 329..332: mod_base = OTHER; note = thymine
  modified_base 340: mod_base = OTHER; note = thymine
  modified_base 346: mod_base = OTHER; note = thymine
  modified_base 351: mod_base = OTHER; note = thymine
  modified_base 355..357: mod_base = OTHER; note = thymine
  modified_base 359..360: mod_base = OTHER; note = thymine
  modified_base 362: mod_base = OTHER; note = thymine
  modified_base 364: mod_base = OTHER; note = thymine
  modified_base 368..369: mod_base = OTHER; note = thymine
  modified_base 382: mod_base = OTHER; note = thymine
  modified_base 384..386: mod_base = OTHER; note = thymine
  modified_base 390..391: mod_base = OTHER; note = thymine
  modified_base 403: mod_base = OTHER; note = thymine
  modified_base 406..407: mod_base = OTHER; note = thymine
  modified_base 410..412: mod_base = OTHER; note = thymine
  modified_base 417: mod_base = OTHER; note = thymine
  modified_base 422..424: mod_base = OTHER; note = thymine
  modified_base 427: mod_base = OTHER; note = thymine
  modified_base 430: mod_base = OTHER; note = thymine
  modified_base 432..433: mod_base = OTHER; note = thymine
  modified_base 438: mod_base = OTHER; note = thymine
  modified_base 440..441: mod_base = OTHER; note = thymine
  modified_base 443..444: mod_base = OTHER; note = thymine
  modified_base 452: mod_base = OTHER; note = thymine
  modified_base 454: mod_base = OTHER; note = thymine
  modified_base 457: mod_base = OTHER; note = thymine
  modified_base 459..460: mod_base = OTHER; note = thymine
  modified_base 462: mod_base = OTHER; note = thymine
  modified_base 465: mod_base = OTHER; note = thymine
  modified_base 472: mod_base = OTHER; note = thymine
  modified_base 481: mod_base = OTHER; note = thymine
  modified_base 483: mod_base = OTHER; note = thymine
  modified_base 488: mod_base = OTHER; note = thymine
  modified_base 491: mod_base = OTHER; note = thymine
  modified_base 493..494: mod_base = OTHER; note = thymine
  modified_base 502: mod_base = OTHER; note = thymine
  modified_base 505: mod_base = OTHER; note = thymine
  modified_base 508: mod_base = OTHER; note = thymine
  modified_base 513..515: mod_base = OTHER; note = thymine
  modified_base 518..519: mod_base = OTHER; note = thymine
  modified_base 521: mod_base = OTHER; note = thymine
  modified_base 523: mod_base = OTHER; note = thymine
  modified_base 527: mod_base = OTHER; note = thymine
  modified_base 530..531: mod_base = OTHER; note = thymine
  modified_base 534: mod_base = OTHER; note = thymine
  modified_base 538: mod_base = OTHER; note = thymine
  modified_base 541: mod_base = OTHER; note = thymine
  modified_base 543: mod_base = OTHER; note = thymine
  modified_base 550: mod_base = OTHER; note = thymine
  modified_base 553..554: mod_base = OTHER; note = thymine
  modified_base 559: mod_base = OTHER; note = thymine
  modified_base 561: mod_base = OTHER; note = thymine
  modified_base 572..574: mod_base = OTHER; note = thymine
  modified_base 576: mod_base = OTHER; note = thymine
  modified_base 578: mod_base = OTHER; note = thymine
  modified_base 580: mod_base = OTHER; note = thymine
  modified_base 589: mod_base = OTHER; note = thymine
  modified_base 605..606: mod_base = OTHER; note = thymine
  modified_base 609: mod_base = OTHER; note = thymine
  modified_base 611..613: mod_base = OTHER; note = thymine
  modified_base 618: mod_base = OTHER; note = thymine
  modified_base 626: mod_base = OTHER; note = thymine
  modified_base 634: mod_base = OTHER; note = thymine
  modified_base 637: mod_base = OTHER; note = thymine
  modified_base 639: mod_base = OTHER; note = thymine
  modified_base 643: mod_base = OTHER; note = thymine
  modified_base 649..650: mod_base = OTHER; note = thymine
  modified_base 652: mod_base = OTHER; note = thymine
  modified_base 655: mod_base = OTHER; note = thymine
  modified_base 662..664: mod_base = OTHER; note = thymine
  modified_base 670: mod_base = OTHER; note = thymine
  modified_base 675: mod_base = OTHER; note = thymine
  modified_base 679: mod_base = OTHER; note = thymine
  modified_base 682..684: mod_base = OTHER; note = thymine
  modified_base 686: mod_base = OTHER; note = thymine
  modified_base 688: mod_base = OTHER; note = thymine
  modified_base 695: mod_base = OTHER; note = thymine
  modified_base 697: mod_base = OTHER; note = thymine
  modified_base 700: mod_base = OTHER; note = thymine
  modified_base 706..707: mod_base = OTHER; note = thymine
  modified_base 710..711: mod_base = OTHER; note = thymine
  modified_base 715: mod_base = OTHER; note = thymine
  modified_base 723: mod_base = OTHER; note = thymine
  modified_base 725: mod_base = OTHER; note = thymine
  modified_base 728..729: mod_base = OTHER; note = thymine
  modified_base 732: mod_base = OTHER; note = thymine
  modified_base 745: mod_base = OTHER; note = thymine
  modified_base 751..752: mod_base = OTHER; note = thymine
  modified_base 757: mod_base = OTHER; note = thymine
  modified_base 759: mod_base = OTHER; note = thymine
  modified_base 764: mod_base = OTHER; note = thymine
  modified_base 766: mod_base = OTHER; note = thymine
  modified_base 771: mod_base = OTHER; note = thymine
  modified_base 780..781: mod_base = OTHER; note = thymine
  modified_base 788..789: mod_base = OTHER; note = thymine
  modified_base 794..795: mod_base = OTHER; note = thymine
  modified_base 798..799: mod_base = OTHER; note = thymine
  modified_base 808: mod_base = OTHER; note = thymine
  modified_base 816..817: mod_base = OTHER; note = thymine
  modified_base 822..823: mod_base = OTHER; note = thymine
  modified_base 833: mod_base = OTHER; note = thymine
  modified_base 836..837: mod_base = OTHER; note = thymine
  modified_base 845..846: mod_base = OTHER; note = thymine
  modified_base 848..849: mod_base = OTHER; note = thymine
  modified_base 851: mod_base = OTHER; note = thymine
  modified_base 854: mod_base = OTHER; note = thymine
  modified_base 857: mod_base = OTHER; note = thymine
  modified_base 859..861: mod_base = OTHER; note = thymine
  modified_base 869: mod_base = OTHER; note = thymine
  modified_base 872: mod_base = OTHER; note = thymine
  modified_base 883: mod_base = OTHER; note = thymine
  modified_base 889: mod_base = OTHER; note = thymine
  modified_base 896: mod_base = OTHER; note = thymine
  modified_base 898..899: mod_base = OTHER; note = thymine
  modified_base 901: mod_base = OTHER; note = thymine
  modified_base 903: mod_base = OTHER; note = thymine
  modified_base 911: mod_base = OTHER; note = thymine
  modified_base 915: mod_base = OTHER; note = thymine
  modified_base 918: mod_base = OTHER; note = thymine
  modified_base 921: mod_base = OTHER; note = thymine
  modified_base 933: mod_base = OTHER; note = thymine
  modified_base 938: mod_base = OTHER; note = thymine
  modified_base 940..943: mod_base = OTHER; note = thymine
  modified_base 949: mod_base = OTHER; note = thymine
  modified_base 952: mod_base = OTHER; note = thymine
  modified_base 955: mod_base = OTHER; note = thymine
  modified_base 957: mod_base = OTHER; note = thymine
  modified_base 965: mod_base = OTHER; note = thymine
  modified_base 970..972: mod_base = OTHER; note = thymine
  modified_base 976: mod_base = OTHER; note = thymine
  modified_base 979: mod_base = OTHER; note = thymine
  modified_base 983: mod_base = OTHER; note = thymine
  modified_base 985: mod_base = OTHER; note = thymine
  modified_base 989..990: mod_base = OTHER; note = thymine
  modified_base 992: mod_base = OTHER; note = thymine
  modified_base 994: mod_base = OTHER; note = thymine
  modified_base 999: mod_base = OTHER; note = thymine
  modified_base 1001..1002: mod_base = OTHER; note = thymine
  modified_base 1008: mod_base = OTHER; note = thymine
  modified_base 1010..1012: mod_base = OTHER; note = thymine
  modified_base 1015..1016: mod_base = OTHER; note = thymine
  modified_base 1027: mod_base = OTHER; note = thymine
  modified_base 1030: mod_base = OTHER; note = thymine
  modified_base 1032: mod_base = OTHER; note = thymine
  modified_base 1041: mod_base = OTHER; note = thymine
  modified_base 1045: mod_base = OTHER; note = thymine
  modified_base 1048: mod_base = OTHER; note = thymine
  modified_base 1054: mod_base = OTHER; note = thymine
  modified_base 1065: mod_base = OTHER; note = thymine
  modified_base 1069: mod_base = OTHER; note = thymine
  modified_base 1071: mod_base = OTHER; note = thymine
  modified_base 1074: mod_base = OTHER; note = thymine
  modified_base 1086: mod_base = OTHER; note = thymine
  modified_base 1090: mod_base = OTHER; note = thymine
  modified_base 1095: mod_base = OTHER; note = thymine
  modified_base 1098: mod_base = OTHER; note = thymine
  modified_base 1100: mod_base = OTHER; note = thymine
  modified_base 1102: mod_base = OTHER; note = thymine
  modified_base 1108..1110: mod_base = OTHER; note = thymine
  modified_base 1112..1113: mod_base = OTHER; note = thymine
  modified_base 1116: mod_base = OTHER; note = thymine
  modified_base 1128..1129: mod_base = OTHER; note = thymine
  modified_base 1137: mod_base = OTHER; note = thymine
  modified_base 1140: mod_base = OTHER; note = thymine
  modified_base 1144: mod_base = OTHER; note = thymine
  modified_base 1146: mod_base = OTHER; note = thymine
  modified_base 1150..1151: mod_base = OTHER; note = thymine
  modified_base 1154: mod_base = OTHER; note = thymine
  modified_base 1159..1160: mod_base = OTHER; note = thymine
  modified_base 1169..1172: mod_base = OTHER; note = thymine
  modified_base 1175: mod_base = OTHER; note = thymine
  modified_base 1177: mod_base = OTHER; note = thymine
  modified_base 1179: mod_base = OTHER; note = thymine
  modified_base 1191..1193: mod_base = OTHER; note = thymine
  modified_base 1205: mod_base = OTHER; note = thymine
  modified_base 1208..1209: mod_base = OTHER; note = thymine
  modified_base 1217: mod_base = OTHER; note = thymine
  modified_base 1220: mod_base = OTHER; note = thymine
  modified_base 1223..1224: mod_base = OTHER; note = thymine
  modified_base 1227: mod_base = OTHER; note = thymine
  modified_base 1241: mod_base = OTHER; note = thymine
  modified_base 1243..1245: mod_base = OTHER; note = thymine
  modified_base 1251: mod_base = OTHER; note = thymine
  modified_base 1253: mod_base = OTHER; note = thymine
  modified_base 1255: mod_base = OTHER; note = thymine
  modified_base 1259..1260: mod_base = OTHER; note = thymine
  modified_base 1264: mod_base = OTHER; note = thymine
  modified_base 1267: mod_base = OTHER; note = thymine
  modified_base 1271: mod_base = OTHER; note = thymine
  modified_base 1275: mod_base = OTHER; note = thymine
  modified_base 1277..1278: mod_base = OTHER; note = thymine
  modified_base 1285: mod_base = OTHER; note = thymine
  modified_base 1289..1290: mod_base = OTHER; note = thymine
  modified_base 1292..1293: mod_base = OTHER; note = thymine
  modified_base 1296..1298: mod_base = OTHER; note = thymine
  modified_base 1300: mod_base = OTHER; note = thymine
  modified_base 1305: mod_base = OTHER; note = thymine
  modified_base 1308: mod_base = OTHER; note = thymine
  modified_base 1319: mod_base = OTHER; note = thymine
  modified_base 1321..1322: mod_base = OTHER; note = thymine
  modified_base 1336: mod_base = OTHER; note = thymine
  modified_base 1342: mod_base = OTHER; note = thymine
  modified_base 1345..1347: mod_base = OTHER; note = thymine
  modified_base 1351..1353: mod_base = OTHER; note = thymine
  modified_base 1356..1357: mod_base = OTHER; note = thymine
  modified_base 1362: mod_base = OTHER; note = thymine
  modified_base 1364: mod_base = OTHER; note = thymine
  modified_base 1366: mod_base = OTHER; note = thymine
  modified_base 1368..1370: mod_base = OTHER; note = thymine
  modified_base 1380: mod_base = OTHER; note = thymine
  modified_base 1382..1383: mod_base = OTHER; note = thymine
  modified_base 1385..1386: mod_base = OTHER; note = thymine
  modified_base 1394: mod_base = OTHER; note = thymine
  modified_base 1398: mod_base = OTHER; note = thymine
  modified_base 1400..1401: mod_base = OTHER; note = thymine
  modified_base 1404..1405: mod_base = OTHER; note = thymine
  modified_base 1409..1410: mod_base = OTHER; note = thymine
  modified_base 1413: mod_base = OTHER; note = thymine
  modified_base 1417: mod_base = OTHER; note = thymine
  modified_base 1419: mod_base = OTHER; note = thymine
  modified_base 1424: mod_base = OTHER; note = thymine
  modified_base 1437: mod_base = OTHER; note = thymine
  modified_base 1451: mod_base = OTHER; note = thymine
  modified_base 1456..1458: mod_base = OTHER; note = thymine
  modified_base 1469..1470: mod_base = OTHER; note = thymine
  modified_base 1478..1479: mod_base = OTHER; note = thymine
  modified_base 1483: mod_base = OTHER; note = thymine
  modified_base 1486..1487: mod_base = OTHER; note = thymine
  modified_base 1493: mod_base = OTHER; note = thymine
  modified_base 1495: mod_base = OTHER; note = thymine
  modified_base 1498: mod_base = OTHER; note = thymine
  modified_base 1502: mod_base = OTHER; note = thymine
  modified_base 1510: mod_base = OTHER; note = thymine
  modified_base 1512: mod_base = OTHER; note = thymine
  modified_base 1527..1529: mod_base = OTHER; note = thymine
  modified_base 1534: mod_base = OTHER; note = thymine
  modified_base 1544: mod_base = OTHER; note = thymine
  modified_base 1551: mod_base = OTHER; note = thymine
  modified_base 1554: mod_base = OTHER; note = thymine
  modified_base 1560: mod_base = OTHER; note = thymine
  modified_base 1579: mod_base = OTHER; note = thymine
  modified_base 1588: mod_base = OTHER; note = thymine
  modified_base 1591: mod_base = OTHER; note = thymine
  modified_base 1593: mod_base = OTHER; note = thymine
  modified_base 1604: mod_base = OTHER; note = thymine
  modified_base 1613: mod_base = OTHER; note = thymine
  modified_base 1624: mod_base = OTHER; note = thymine
  modified_base 1635: mod_base = OTHER; note = thymine
  modified_base 1638..1639: mod_base = OTHER; note = thymine
  modified_base 1644: mod_base = OTHER; note = thymine
  modified_base 1648: mod_base = OTHER; note = thymine
  modified_base 1653: mod_base = OTHER; note = thymine
  modified_base 1657..1658: mod_base = OTHER; note = thymine
  modified_base 1676: mod_base = OTHER; note = thymine
  modified_base 1680: mod_base = OTHER; note = thymine
  modified_base 1683: mod_base = OTHER; note = thymine
  modified_base 1686: mod_base = OTHER; note = thymine
  modified_base 1692: mod_base = OTHER; note = thymine
  modified_base 1694: mod_base = OTHER; note = thymine
  modified_base 1698..1699: mod_base = OTHER; note = thymine
  modified_base 1702: mod_base = OTHER; note = thymine
  modified_base 1705: mod_base = OTHER; note = thymine
  modified_base 1708: mod_base = OTHER; note = thymine
  modified_base 1714..1715: mod_base = OTHER; note = thymine
  modified_base 1720: mod_base = OTHER; note = thymine
  modified_base 1722: mod_base = OTHER; note = thymine
  modified_base 1728: mod_base = OTHER; note = thymine
  modified_base 1732: mod_base = OTHER; note = thymine
  modified_base 1739..1740: mod_base = OTHER; note = thymine
  modified_base 1742: mod_base = OTHER; note = thymine
  modified_base 1747: mod_base = OTHER; note = thymine
  modified_base 1749: mod_base = OTHER; note = thymine
  modified_base 1762: mod_base = OTHER; note = thymine
  modified_base 1764: mod_base = OTHER; note = thymine
  modified_base 1767..1768: mod_base = OTHER; note = thymine
  modified_base 1772: mod_base = OTHER; note = thymine
  modified_base 1783..1784: mod_base = OTHER; note = thymine
  modified_base 1793: mod_base = OTHER; note = thymine
  modified_base 1795: mod_base = OTHER; note = thymine
  modified_base 1798..1801: mod_base = OTHER; note = thymine
  modified_base 1805: mod_base = OTHER; note = thymine
  modified_base 1807..1811: mod_base = OTHER; note = thymine
  modified_base 1813..1814: mod_base = OTHER; note = thymine
  modified_base 1816..1817: mod_base = OTHER; note = thymine
  modified_base 1827: mod_base = OTHER; note = thymine
  modified_base 1831..1832: mod_base = OTHER; note = thymine
  modified_base 1834: mod_base = OTHER; note = thymine
  modified_base 1837: mod_base = OTHER; note = thymine
  modified_base 1839..1841: mod_base = OTHER; note = thymine
  modified_base 1845: mod_base = OTHER; note = thymine
  modified_base 1849: mod_base = OTHER; note = thymine
  modified_base 1855: mod_base = OTHER; note = thymine
  modified_base 1857..1859: mod_base = OTHER; note = thymine
  modified_base 1861: mod_base = OTHER; note = thymine
  modified_base 1870: mod_base = OTHER; note = thymine
  modified_base 1872..1873: mod_base = OTHER; note = thymine
  modified_base 1876: mod_base = OTHER; note = thymine
  modified_base 1882: mod_base = OTHER; note = thymine
  modified_base 1886: mod_base = OTHER; note = thymine
  modified_base 1890..1891: mod_base = OTHER; note = thymine
  modified_base 1893..1894: mod_base = OTHER; note = thymine
  modified_base 1899: mod_base = OTHER; note = thymine
  modified_base 1901: mod_base = OTHER; note = thymine
  modified_base 1906..1907: mod_base = OTHER; note = thymine
  modified_base 1912: mod_base = OTHER; note = thymine
  modified_base 1920..1922: mod_base = OTHER; note = thymine
  modified_base 1927: mod_base = OTHER; note = thymine
  modified_base 1929: mod_base = OTHER; note = thymine
  modified_base 1931..1932: mod_base = OTHER; note = thymine
  modified_base 1934..1935: mod_base = OTHER; note = thymine
  modified_base 1939: mod_base = OTHER; note = thymine
  modified_base 1945: mod_base = OTHER; note = thymine
  modified_base 1954..1955: mod_base = OTHER; note = thymine
  modified_base 1958: mod_base = OTHER; note = thymine
  modified_base 1961: mod_base = OTHER; note = thymine
  modified_base 1963: mod_base = OTHER; note = thymine
  modified_base 1966: mod_base = OTHER; note = thymine
  modified_base 1971: mod_base = OTHER; note = thymine
  modified_base 1974..1975: mod_base = OTHER; note = thymine
  modified_base 1979: mod_base = OTHER; note = thymine
  modified_base 1982: mod_base = OTHER; note = thymine
  modified_base 1991: mod_base = OTHER; note = thymine
  modified_base 1993: mod_base = OTHER; note = thymine
  modified_base 1995: mod_base = OTHER; note = thymine
  modified_base 2012: mod_base = OTHER; note = thymine
  modified_base 2017: mod_base = OTHER; note = thymine
  modified_base 2021: mod_base = OTHER; note = thymine
  modified_base 2028: mod_base = OTHER; note = thymine
  modified_base 2031: mod_base = OTHER; note = thymine
  modified_base 2036..2037: mod_base = OTHER; note = thymine
  modified_base 2041: mod_base = OTHER; note = thymine
  modified_base 2043..2044: mod_base = OTHER; note = thymine
  modified_base 2046: mod_base = OTHER; note = thymine
  modified_base 2048: mod_base = OTHER; note = thymine
  modified_base 2050: mod_base = OTHER; note = thymine
  modified_base 2053..2054: mod_base = OTHER; note = thymine
  modified_base 2057..2058: mod_base = OTHER; note = thymine
  modified_base 2063: mod_base = OTHER; note = thymine
  modified_base 2068: mod_base = OTHER; note = thymine
  modified_base 2074: mod_base = OTHER; note = thymine
  modified_base 2076: mod_base = OTHER; note = thymine
  modified_base 2078: mod_base = OTHER; note = thymine
  modified_base 2081: mod_base = OTHER; note = thymine
  modified_base 2086..2090: mod_base = OTHER; note = thymine
  modified_base 2097..2099: mod_base = OTHER; note = thymine
  modified_base 2101: mod_base = OTHER; note = thymine
  modified_base 2104: mod_base = OTHER; note = thymine
  modified_base 2107: mod_base = OTHER; note = thymine
  modified_base 2109: mod_base = OTHER; note = thymine
  modified_base 2112..2115: mod_base = OTHER; note = thymine
  modified_base 2117: mod_base = OTHER; note = thymine
  modified_base 2125: mod_base = OTHER; note = thymine
  modified_base 2129: mod_base = OTHER; note = thymine
  modified_base 2131: mod_base = OTHER; note = thymine
  modified_base 2134: mod_base = OTHER; note = thymine
  modified_base 2137..2138: mod_base = OTHER; note = thymine
  modified_base 2142: mod_base = OTHER; note = thymine
  modified_base 2148..2149: mod_base = OTHER; note = thymine
  modified_base 2154: mod_base = OTHER; note = thymine
  modified_base 2157: mod_base = OTHER; note = thymine
  modified_base 2162: mod_base = OTHER; note = thymine
  modified_base 2164: mod_base = OTHER; note = thymine
  modified_base 2173: mod_base = OTHER; note = thymine
  modified_base 2177: mod_base = OTHER; note = thymine
  modified_base 2181: mod_base = OTHER; note = thymine
  modified_base 2184: mod_base = OTHER; note = thymine
  modified_base 2187..2188: mod_base = OTHER; note = thymine
  modified_base 2192: mod_base = OTHER; note = thymine
  modified_base 2194: mod_base = OTHER; note = thymine
  modified_base 2207: mod_base = OTHER; note = thymine
  modified_base 2209..2210: mod_base = OTHER; note = thymine
  modified_base 2213: mod_base = OTHER; note = thymine
  modified_base 2218: mod_base = OTHER; note = thymine
  modified_base 2223..2225: mod_base = OTHER; note = thymine
  modified_base 2232: mod_base = OTHER; note = thymine
  modified_base 2236: mod_base = OTHER; note = thymine
  modified_base 2238: mod_base = OTHER; note = thymine
  modified_base 2240: mod_base = OTHER; note = thymine
  modified_base 2243: mod_base = OTHER; note = thymine
  modified_base 2250..2251: mod_base = OTHER; note = thymine
  modified_base 2253..2254: mod_base = OTHER; note = thymine
  modified_base 2256: mod_base = OTHER; note = thymine
  modified_base 2258: mod_base = OTHER; note = thymine
  modified_base 2260: mod_base = OTHER; note = thymine
  modified_base 2262: mod_base = OTHER; note = thymine
  modified_base 2264: mod_base = OTHER; note = thymine
  modified_base 2269..2270: mod_base = OTHER; note = thymine
  modified_base 2277: mod_base = OTHER; note = thymine
  modified_base 2279: mod_base = OTHER; note = thymine
  modified_base 2284..2285: mod_base = OTHER; note = thymine
  modified_base 2287: mod_base = OTHER; note = thymine
  modified_base 2293..2299: mod_base = OTHER; note = thymine
  modified_base 2305: mod_base = OTHER; note = thymine
  modified_base 2307: mod_base = OTHER; note = thymine
  modified_base 2309: mod_base = OTHER; note = thymine
  modified_base 2322: mod_base = OTHER; note = thymine
  modified_base 2325..2327: mod_base = OTHER; note = thymine
  modified_base 2330..2331: mod_base = OTHER; note = thymine
  modified_base 2333: mod_base = OTHER; note = thymine
  modified_base 2338: mod_base = OTHER; note = thymine
  modified_base 2342: mod_base = OTHER; note = thymine
  modified_base 2345..2346: mod_base = OTHER; note = thymine
  modified_base 2348: mod_base = OTHER; note = thymine
  modified_base 2350..2351: mod_base = OTHER; note = thymine
  modified_base 2353: mod_base = OTHER; note = thymine
  modified_base 2358: mod_base = OTHER; note = thymine
  modified_base 2363: mod_base = OTHER; note = thymine
  modified_base 2367: mod_base = OTHER; note = thymine
  modified_base 2369: mod_base = OTHER; note = thymine
  modified_base 2372..2374: mod_base = OTHER; note = thymine
  modified_base 2376: mod_base = OTHER; note = thymine
  modified_base 2379: mod_base = OTHER; note = thymine
  modified_base 2381: mod_base = OTHER; note = thymine
  modified_base 2390: mod_base = OTHER; note = thymine
  modified_base 2392: mod_base = OTHER; note = thymine
  modified_base 2404: mod_base = OTHER; note = thymine
  modified_base 2421..2423: mod_base = OTHER; note = thymine
  modified_base 2425: mod_base = OTHER; note = thymine
  modified_base 2434..2437: mod_base = OTHER; note = thymine
  modified_base 2440: mod_base = OTHER; note = thymine
  modified_base 2446..2448: mod_base = OTHER; note = thymine
  modified_base 2450: mod_base = OTHER; note = thymine
  modified_base 2452..2453: mod_base = OTHER; note = thymine
  modified_base 2457: mod_base = OTHER; note = thymine
  modified_base 2460..2462: mod_base = OTHER; note = thymine
  modified_base 2464: mod_base = OTHER; note = thymine
  modified_base 2467: mod_base = OTHER; note = thymine
  modified_base 2471: mod_base = OTHER; note = thymine
  modified_base 2477: mod_base = OTHER; note = thymine
  modified_base 2481..2482: mod_base = OTHER; note = thymine
  modified_base 2484: mod_base = OTHER; note = thymine
  modified_base 2486..2487: mod_base = OTHER; note = thymine
  modified_base 2490..2492: mod_base = OTHER; note = thymine
  modified_base 2497: mod_base = OTHER; note = thymine
  modified_base 2501: mod_base = OTHER; note = thymine
  modified_base 2504..2505: mod_base = OTHER; note = thymine
  modified_base 2508..2511: mod_base = OTHER; note = thymine
  modified_base 2513: mod_base = OTHER; note = thymine
  modified_base 2527..2530: mod_base = OTHER; note = thymine
  modified_base 2532: mod_base = OTHER; note = thymine
  modified_base 2537: mod_base = OTHER; note = thymine
  modified_base 2541: mod_base = OTHER; note = thymine
  modified_base 2543: mod_base = OTHER; note = thymine
  modified_base 2546..2547: mod_base = OTHER; note = thymine
  modified_base 2549..2551: mod_base = OTHER; note = thymine
  modified_base 2558: mod_base = OTHER; note = thymine
  modified_base 2560..2562: mod_base = OTHER; note = thymine
  modified_base 2569: mod_base = OTHER; note = thymine
  modified_base 2576: mod_base = OTHER; note = thymine
  modified_base 2580: mod_base = OTHER; note = thymine
  modified_base 2583: mod_base = OTHER; note = thymine
  modified_base 2590..2592: mod_base = OTHER; note = thymine
  modified_base 2595: mod_base = OTHER; note = thymine
  modified_base 2601: mod_base = OTHER; note = thymine
  modified_base 2609: mod_base = OTHER; note = thymine
  modified_base 2614..2615: mod_base = OTHER; note = thymine
  modified_base 2617: mod_base = OTHER; note = thymine
  modified_base 2627: mod_base = OTHER; note = thymine
  modified_base 2629..2630: mod_base = OTHER; note = thymine
  modified_base 2633: mod_base = OTHER; note = thymine
  modified_base 2639..2641: mod_base = OTHER; note = thymine
  modified_base 2644: mod_base = OTHER; note = thymine
  modified_base 2646..2648: mod_base = OTHER; note = thymine
  modified_base 2651: mod_base = OTHER; note = thymine
  modified_base 2657: mod_base = OTHER; note = thymine
  modified_base 2659: mod_base = OTHER; note = thymine
  modified_base 2668: mod_base = OTHER; note = thymine
  modified_base 2671: mod_base = OTHER; note = thymine
  modified_base 2673..2674: mod_base = OTHER; note = thymine
  modified_base 2677: mod_base = OTHER; note = thymine
  modified_base 2681: mod_base = OTHER; note = thymine
  modified_base 2685: mod_base = OTHER; note = thymine
  modified_base 2687: mod_base = OTHER; note = thymine
  modified_base 2689: mod_base = OTHER; note = thymine
  modified_base 2695: mod_base = OTHER; note = thymine
  modified_base 2699..2700: mod_base = OTHER; note = thymine
  modified_base 2704..2706: mod_base = OTHER; note = thymine
  modified_base 2710: mod_base = OTHER; note = thymine
  modified_base 2713..2714: mod_base = OTHER; note = thymine
  modified_base 2720: mod_base = OTHER; note = thymine
  modified_base 2722: mod_base = OTHER; note = thymine
  modified_base 2724: mod_base = OTHER; note = thymine
  modified_base 2736: mod_base = OTHER; note = thymine
  modified_base 2741..2742: mod_base = OTHER; note = thymine
  modified_base 2748..2749: mod_base = OTHER; note = thymine
  modified_base 2751: mod_base = OTHER; note = thymine
  modified_base 2753: mod_base = OTHER; note = thymine
  modified_base 2759: mod_base = OTHER; note = thymine
  modified_base 2761..2762: mod_base = OTHER; note = thymine
  modified_base 2765..2766: mod_base = OTHER; note = thymine
  modified_base 2768: mod_base = OTHER; note = thymine
  modified_base 2770: mod_base = OTHER; note = thymine
  modified_base 2775: mod_base = OTHER; note = thymine
  modified_base 2778..2779: mod_base = OTHER; note = thymine
  modified_base 2786: mod_base = OTHER; note = thymine
  modified_base 2789: mod_base = OTHER; note = thymine
  modified_base 2793: mod_base = OTHER; note = thymine
  modified_base 2798..2799: mod_base = OTHER; note = thymine
  modified_base 2801: mod_base = OTHER; note = thymine
  modified_base 2805: mod_base = OTHER; note = thymine
  modified_base 2824: mod_base = OTHER; note = thymine
  modified_base 2827..2828: mod_base = OTHER; note = thymine
  modified_base 2830..2832: mod_base = OTHER; note = thymine
  modified_base 2836..2838: mod_base = OTHER; note = thymine
  modified_base 2845: mod_base = OTHER; note = thymine
  modified_base 2847..2849: mod_base = OTHER; note = thymine
  modified_base 2854: mod_base = OTHER; note = thymine
  modified_base 2859: mod_base = OTHER; note = thymine
  modified_base 2861: mod_base = OTHER; note = thymine
  modified_base 2864: mod_base = OTHER; note = thymine
  modified_base 2875..2877: mod_base = OTHER; note = thymine
  modified_base 2885: mod_base = OTHER; note = thymine
  modified_base 2889..2890: mod_base = OTHER; note = thymine
  modified_base 2893: mod_base = OTHER; note = thymine
  modified_base 2895..2896: mod_base = OTHER; note = thymine
  modified_base 2901: mod_base = OTHER; note = thymine
  modified_base 2907: mod_base = OTHER; note = thymine
  modified_base 2909..2910: mod_base = OTHER; note = thymine
  modified_base 2912: mod_base = OTHER; note = thymine
  modified_base 2916: mod_base = OTHER; note = thymine
  modified_base 2918: mod_base = OTHER; note = thymine
  modified_base 2920: mod_base = OTHER; note = thymine
  modified_base 2922: mod_base = OTHER; note = thymine
  modified_base 2925: mod_base = OTHER; note = thymine
  modified_base 2927..2930: mod_base = OTHER; note = thymine
  modified_base 2937..2938: mod_base = OTHER; note = thymine
  modified_base 2940: mod_base = OTHER; note = thymine
  modified_base 2944: mod_base = OTHER; note = thymine
  modified_base 2953..2954: mod_base = OTHER; note = thymine
  modified_base 2960: mod_base = OTHER; note = thymine
  modified_base 2962: mod_base = OTHER; note = thymine
  modified_base 2971: mod_base = OTHER; note = thymine
  modified_base 2979: mod_base = OTHER; note = thymine
  modified_base 2981: mod_base = OTHER; note = thymine
  modified_base 2983: mod_base = OTHER; note = thymine
  modified_base 2985: mod_base = OTHER; note = thymine
  modified_base 2988: mod_base = OTHER; note = thymine
  modified_base 2990..2991: mod_base = OTHER; note = thymine
  modified_base 2993..2994: mod_base = OTHER; note = thymine
  modified_base 2996: mod_base = OTHER; note = thymine
  modified_base 2998..2999: mod_base = OTHER; note = thymine
  modified_base 3001..3006: mod_base = OTHER; note = thymine
  modified_base 3009..3011: mod_base = OTHER; note = thymine
  modified_base 3013: mod_base = OTHER; note = thymine
  modified_base 3019: mod_base = OTHER; note = thymine
  modified_base 3031: mod_base = OTHER; note = thymine
  modified_base 3035: mod_base = OTHER; note = thymine
  modified_base 3039..3040: mod_base = OTHER; note = thymine
  modified_base 3043: mod_base = OTHER; note = thymine
  modified_base 3047: mod_base = OTHER; note = thymine
  modified_base 3055: mod_base = OTHER; note = thymine
  modified_base 3057: mod_base = OTHER; note = thymine
  modified_base 3064..3065: mod_base = OTHER; note = thymine
  modified_base 3072: mod_base = OTHER; note = thymine
  modified_base 3074: mod_base = OTHER; note = thymine
  modified_base 3076..3078: mod_base = OTHER; note = thymine
  modified_base 3081..3082: mod_base = OTHER; note = thymine
  modified_base 3089: mod_base = OTHER; note = thymine
  modified_base 3092: mod_base = OTHER; note = thymine
  modified_base 3095: mod_base = OTHER; note = thymine
  modified_base 3101: mod_base = OTHER; note = thymine
  modified_base 3105: mod_base = OTHER; note = thymine
  modified_base 3107: mod_base = OTHER; note = thymine
  modified_base 3109: mod_base = OTHER; note = thymine
  modified_base 3115: mod_base = OTHER; note = thymine
  modified_base 3117..3119: mod_base = OTHER; note = thymine
  modified_base 3121..3122: mod_base = OTHER; note = thymine
  modified_base 3127: mod_base = OTHER; note = thymine
  modified_base 3131: mod_base = OTHER; note = thymine
  modified_base 3136..3137: mod_base = OTHER; note = thymine
  modified_base 3139..3140: mod_base = OTHER; note = thymine
  modified_base 3145: mod_base = OTHER; note = thymine
  modified_base 3147..3148: mod_base = OTHER; note = thymine
  modified_base 3150..3151: mod_base = OTHER; note = thymine
  modified_base 3157..3161: mod_base = OTHER; note = thymine
  modified_base 3164: mod_base = OTHER; note = thymine
  modified_base 3166..3168: mod_base = OTHER; note = thymine
  modified_base 3170: mod_base = OTHER; note = thymine
  modified_base 3174: mod_base = OTHER; note = thymine
  modified_base 3181: mod_base = OTHER; note = thymine
  modified_base 3187: mod_base = OTHER; note = thymine
  modified_base 3189: mod_base = OTHER; note = thymine
  modified_base 3195..3196: mod_base = OTHER; note = thymine
  modified_base 3199..3200: mod_base = OTHER; note = thymine
  modified_base 3205: mod_base = OTHER; note = thymine
  modified_base 3209: mod_base = OTHER; note = thymine
  modified_base 3212: mod_base = OTHER; note = thymine
  modified_base 3214: mod_base = OTHER; note = thymine
  modified_base 3221..3222: mod_base = OTHER; note = thymine
  modified_base 3226..3227: mod_base = OTHER; note = thymine
  modified_base 3232: mod_base = OTHER; note = thymine
  modified_base 3235..3236: mod_base = OTHER; note = thymine
  modified_base 3244: mod_base = OTHER; note = thymine
  modified_base 3246: mod_base = OTHER; note = thymine
  modified_base 3250..3251: mod_base = OTHER; note = thymine
  modified_base 3257: mod_base = OTHER; note = thymine
  modified_base 3259: mod_base = OTHER; note = thymine
  modified_base 3263: mod_base = OTHER; note = thymine
  modified_base 3267..3268: mod_base = OTHER; note = thymine
  modified_base 3272: mod_base = OTHER; note = thymine
  modified_base 3279: mod_base = OTHER; note = thymine
  modified_base 3281: mod_base = OTHER; note = thymine
  modified_base 3284..3285: mod_base = OTHER; note = thymine
  modified_base 3292: mod_base = OTHER; note = thymine
  modified_base 3296: mod_base = OTHER; note = thymine
  modified_base 3300: mod_base = OTHER; note = thymine
  modified_base 3302: mod_base = OTHER; note = thymine
  modified_base 3308..3309: mod_base = OTHER; note = thymine
  modified_base 3316: mod_base = OTHER; note = thymine
  modified_base 3319..3320: mod_base = OTHER; note = thymine
  modified_base 3325: mod_base = OTHER; note = thymine
  modified_base 3327: mod_base = OTHER; note = thymine
  modified_base 3332: mod_base = OTHER; note = thymine
  modified_base 3341..3342: mod_base = OTHER; note = thymine
  modified_base 3344: mod_base = OTHER; note = thymine
  modified_base 3347: mod_base = OTHER; note = thymine
  modified_base 3350..3353: mod_base = OTHER; note = thymine
  modified_base 3356: mod_base = OTHER; note = thymine
  modified_base 3358: mod_base = OTHER; note = thymine
  modified_base 3363: mod_base = OTHER; note = thymine
  modified_base 3368..3369: mod_base = OTHER; note = thymine
  modified_base 3372: mod_base = OTHER; note = thymine
  modified_base 3376: mod_base = OTHER; note = thymine
  modified_base 3382: mod_base = OTHER; note = thymine
  modified_base 3384..3385: mod_base = OTHER; note = thymine
  modified_base 3392: mod_base = OTHER; note = thymine
  modified_base 3394: mod_base = OTHER; note = thymine
  modified_base 3398: mod_base = OTHER; note = thymine
  modified_base 3403: mod_base = OTHER; note = thymine
  modified_base 3408: mod_base = OTHER; note = thymine
  modified_base 3410: mod_base = OTHER; note = thymine
  misc_feature 1..3413: note = Junin virus strain Candid-1 segment S - AY746353 - virusesM> ssrna negative-strand virusesM> arenaviridaeM> arenavirusM> new world arenaviruses
  source 1..3413: mol_type = other RNA
gtgcagtaaggggatcctaggcgattttggttacgctataattgtaactgttttctgtttggacaacatcaaaaacatccattgcacaatggggcagttcattagcttcatgcaagaaataccaacctttttgcaggaggctctgaacattgctcttgttgcagtcagtctcattgccatcattaagggtatagtgaacttgtacaaaagtggtttattccaattctttgtattcctagcgcttgcaggaagatcctgcacagaagaagctttcaaaatcggactgcacactgagttccagactgtgtccttctcaatggtgggtctcttttccaacaatccacatgacctacctttgttgtgtaccttaaacaagagccatctttacattaaggggggcaatgcttcatttcagatcagctttgatgatattgcagtattgttgccacagtatgatgttataatacaacatccagcagatatgagctggtgttccaaaagtgatgatcaaatttggttgtctcagtggttcatgaatgctgtggggcatgattggcatctagacccaccatttctgtgtaggaaccgtgcaaagacagaaggcttcatctttcaagtcaacacctccaagactggtgtcaatggaaattatgctaagaagtttaagactggcatgcatcatttatatagagaatatcctgacccttgcttgaatggcaaactgtgcttaatgaaggcacaacctaccagttggcctctccaatgtccactcgaccacgttaacacattacacttccttacaagaggtaaaaacattcaacttccaaggaggtccttgaaagcattcttctcctggtctttgacagactcatccggcaaggatacccctggaggctattgtctagaagagtggatgctcgtagcagccaaaatgaagtgttttggcaatactgctgtagcaaaatgcaatttgaatcatgactctgaattctgtgacatgttgaggctctttgattacaacaaaaatgctatcaaaaccctaaatgatgaaactaagaaacaagtaaatctgatggggcagacaatcaatgccctgatatctgacaatttattgatgaaaaacaaaattagggaactgatgagtgtcccttactgcaattacacaaaattttggtatgtcaaccacacactttcaggacaacactcattaccaaggtgctggttaataaaaaacaacagctatttgaacatctctgacttccgtaatgactggatattagaaagtgacttcttaatttctgaaatgctaagcaaagagtattcggacaggcagggtaaaactcctttgactttagttgacatctgtatttggagcacagtattcttcacagcgtcactcttccttcacttggtgggtataccctcccacagacacatcaggggcgaagcatgccctttgccacacaggttgaacagcttgggtggttgcagatgtggtaagtaccccaatctaaagaaaccaacagtttggcgtagaggacactaagacctcctgagggtccccaccagcccgggcactgcccgggctggtgtggccccccagtccgcggcctggccgcggactggggaggcactgcttacagtgcataggctgccttcgggaggaacagcaagctcggtggtaatagaggtgtaggttcctcctcatagagcttcccatctagcactgactgaaacattatgcagtctagcagagcacagtgtggttcactggaggccaacttgaagggagtatccttttccctctttttcttattgacaaccactccattgtgatatttgcataagtgaccatatttctcccagacctgttgatcaaactgcctggcttgttcagatgtgagcttaacatcaaccagtttaagatctcttcttccatggaggtcaaacaacttcctgatgtcatcggatccttgagtagtcacaaccatgtctggaggcagcaagccgatcacgtaactaagaactcctggcattgcatcttctatgtccttcattaagatgccgtgagagtgtctgctaccatttttaaaccctttctcatcatgtggttttctgaagcagtgaatgtactgcttacctgcaggttggaataatgccatctcaacagggtcagtggctggtccttcaatgtcgagccaaagggtgttggtggggtcgagtttccccactgcctctctgatgacagcttcttgtatctctgtcaagttagccaatctcaaattctgaccgtttttttccggctgtctaggaccagcaactggtttccttgtcagatcaatacttgtgttgtcccatgacctgcctgtgatttgtgatctagaaccaatataaggccaaccatcgccagaaagacaaagtttgtacaaaaggttttcataaggatttctattgcctggtttctcatcaataaacatgccttctcttcgtttaacctgaatggttgattttatgagggaagagaagttttctggggtgactctgattgtttccaacatgtttccaccatcaagaatagatgctccagcctttactgcagctgaaagactgaagttgtaaccagaaatattgatggagctttcatctttagtcacaatctgaaggcagtcatgttcctgagtcagtctgtcaaggtcacttaagtttggatacttcacagtgtatagaagcccaagtgaggttaaagcttgtatgacactgttcattgtctcacctccttgaacagtcatgcatgcaattgtcaatgcaggaacagagccaaactgattgtttagctttgaagggtctttaacatcccatatcctcaccacaccatttcccccagtcccttgctgttgaaatcccagtgttctcaatatctctgatcttttagcaagttgtgactgggacaagttacccatgtaaaccccctgagagcctgtctctgctcttcttatcttgttttttaatttctcaaggtcagacgccaactccatcagttcatccctccccagatctcccaccttgaaaactgtgtttcgttgaacactcctcatggacatgagtctgtcaacctctttattcaggtccctcaacttgttgaggtcttcttccccctttttagtctttctgagtgcccgctgcacctgtgccacttggttgaagtcgatgctgtcagcaattagcttggcgtccttcaaaacatctgacttgacagtctgagtgaattggctcaaacctctccttaaggactgagtccatctaaagcttggaacctccttggagtgtgccatgccagaagttctggtgattttgatctagaatagagttgctcagtgaaagtgttagacactatgcctaggatccactgtgcg

SEQ ID NO: 2 - RNA, 7114 nt, unidentified
  modified_base 8: mod_base = OTHER; note = thymine
  modified_base 12: mod_base = OTHER; note = thymine
  modified_base 15: mod_base = OTHER; note = thymine
  modified_base 21: mod_base = OTHER; note = thymine
  modified_base 25..26: mod_base = OTHER; note = thymine
  modified_base 29: mod_base = OTHER; note = thymine
  modified_base 32..33: mod_base = OTHER; note = thymine
  modified_base 38: mod_base = OTHER; note = thymine
  modified_base 40: mod_base = OTHER; note = thymine
  modified_base 45..46: mod_base = OTHER; note = thymine
  modified_base 48: mod_base = OTHER; note = thymine
  modified_base 51: mod_base = OTHER; note = thymine
  modified_base 53: mod_base = OTHER; note = thymine
  modified_base 57: mod_base = OTHER; note = thymine
  modified_base 59: mod_base = OTHER; note = thymine
  modified_base 63..64: mod_base = OTHER; note = thymine
  modified_base 67: mod_base = OTHER; note = thymine
  modified_base 85: mod_base = OTHER; note = thymine
  modified_base 93: mod_base = OTHER; note = thymine
  modified_base 105: mod_base = OTHER; note = thymine
  modified_base 111: mod_base = OTHER; note = thymine
  modified_base 113: mod_base = OTHER; note = thymine
  modified_base 126: mod_base = OTHER; note = thymine
  modified_base 129: mod_base = OTHER; note = thymine
  modified_base 156..158: mod_base = OTHER; note = thymine
  modified_base 166: mod_base = OTHER; note = thymine
  modified_base 170: mod_base = OTHER; note = thymine
  modified_base 179: mod_base = OTHER; note = thymine
  modified_base 181: mod_base = OTHER; note = thymine
  modified_base 183: mod_base = OTHER; note = thymine
  modified_base 185: mod_base = OTHER; note = thymine
  modified_base 188: mod_base = OTHER; note = thymine
  modified_base 192: mod_base = OTHER; note = thymine
  modified_base 194: mod_base = OTHER; note = thymine
  modified_base 198: mod_base = OTHER; note = thymine
  modified_base 200: mod_base = OTHER; note = thymine
  modified_base 204: mod_base = OTHER; note = thymine
  modified_base 207: mod_base = OTHER; note = thymine
  modified_base 210: mod_base = OTHER; note = thymine
  modified_base 213..215: mod_base = OTHER; note = thymine
  modified_base 218: mod_base = OTHER; note = thymine
  modified_base 221: mod_base = OTHER; note = thymine
  modified_base 227..229: mod_base = OTHER; note = thymine
  modified_base 232: mod_base = OTHER; note = thymine
  modified_base 237: mod_base = OTHER; note = thymine
  modified_base 239: mod_base = OTHER; note = thymine
  modified_base 242: mod_base = OTHER; note = thymine
  modified_base 245: mod_base = OTHER; note = thymine
  modified_base 249: mod_base = OTHER; note = thymine
  modified_base 253..255: mod_base = OTHER; note = thymine
  modified_base 257..259: mod_base = OTHER; note = thymine
  modified_base 264: mod_base = OTHER; note = thymine
  modified_base 269: mod_base = OTHER; note = thymine
  modified_base 275: mod_base = OTHER; note = thymine
  modified_base 277: mod_base = OTHER; note = thymine
  modified_base 279..280: mod_base = OTHER; note = thymine
  modified_base 287..288: mod_base = OTHER; note = thymine
  modified_base 293: mod_base = OTHER; note = thymine
  modified_base 295: mod_base = OTHER; note = thymine
  modified_base 297: mod_base = OTHER; note = thymine
  modified_base 302: mod_base = OTHER; note = thymine
  modified_base 304: mod_base = OTHER; note = thymine
  modified_base 306: mod_base = OTHER; note = thymine
  modified_base 309: mod_base = OTHER; note = thymine
  modified_base 319: mod_base = OTHER; note = thymine
  modified_base 331: mod_base = OTHER; note = thymine
  modified_base 337: mod_base = OTHER; note = thymine
  modified_base 343: mod_base = OTHER; note = thymine
  modified_base 366: mod_base = OTHER; note = thymine
  modified_base 375: mod_base = OTHER; note = thymine
  modified_base 384: mod_base = OTHER; note = thymine
  modified_base 412: mod_base = OTHER; note = thymine
  modified_base 425: mod_base = OTHER; note = thymine
  modified_base 442: mod_base = OTHER; note = thymine
  modified_base 445: mod_base = OTHER; note = thymine
  modified_base 452..453: mod_base = OTHER; note = thymine
  modified_base 456: mod_base = OTHER; note = thymine
  modified_base 460: mod_base = OTHER; note = thymine
  modified_base 462: mod_base = OTHER; note = thymine
  modified_base 464..466: mod_base = OTHER; note = thymine
  modified_base 469: mod_base = OTHER; note = thymine
  modified_base 471: mod_base = OTHER; note = thymine
  modified_base 476..478: mod_base = OTHER; note = thymine
  modified_base 480: mod_base = OTHER; note = thymine
  modified_base 483..484: mod_base = OTHER; note = thymine
  modified_base 488: mod_base = OTHER; note = thymine
  modified_base 493: mod_base = OTHER; note = thymine
  modified_base 497: mod_base = OTHER; note = thymine
  modified_base 499: mod_base = OTHER; note = thymine
  modified_base 506..508: mod_base = OTHER; note = thymine
  modified_base 512: mod_base = OTHER; note = thymine
  modified_base 514: mod_base = OTHER; note = thymine
  modified_base 519: mod_base = OTHER; note = thymine
  modified_base 524..525: mod_base = OTHER; note = thymine
  modified_base 531: mod_base = OTHER; note = thymine
  modified_base 533: mod_base = OTHER; note = thymine
  modified_base 536: mod_base = OTHER; note = thymine
  modified_base 538: mod_base = OTHER; note = thymine
  modified_base 546: mod_base = OTHER; note = thymine
  modified_base 549..551: mod_base = OTHER; note = thymine
  modified_base 557: mod_base = OTHER; note = thymine
  modified_base 559: mod_base = OTHER; note = thymine
  modified_base 562: mod_base = OTHER; note = thymine
  modified_base 566: mod_base = OTHER; note = thymine
  modified_base 573..574: mod_base = OTHER; note = thymine
  modified_base 579..581: mod_base = OTHER; note = thymine
  modified_base 587..588: mod_base = OTHER; note = thymine
  modified_base 597: mod_base = OTHER; note = thymine
  modified_base 605..607: mod_base = OTHER; note = thymine
  modified_base 609: mod_base = OTHER; note = thymine
  modified_base 614: mod_base = OTHER; note = thymine
  modified_base 622: mod_base = OTHER; note = thymine
  modified_base 627: mod_base = OTHER; note = thymine
  modified_base 630: mod_base = OTHER; note = thymine
  modified_base 632..634: mod_base = OTHER; note = thymine
  modified_base 636: mod_base = OTHER; note = thymine
  modified_base 638: mod_base = OTHER; note = thymine
  modified_base 642: mod_base = OTHER; note = thymine
  modified_base 645: mod_base = OTHER; note = thymine
  modified_base 660: mod_base = OTHER; note = thymine
  modified_base 663: mod_base = OTHER; note = thymine
  modified_base 665..666: mod_base = OTHER; note = thymine
  modified_base 669: mod_base = OTHER; note = thymine
  modified_base 674: mod_base = OTHER; note = thymine
  modified_base 677: mod_base = OTHER; note = thymine
  modified_base 679..680: mod_base = OTHER; note = thymine
  modified_base 682..684: mod_base = OTHER; note = thymine
  modified_base 686: mod_base = OTHER; note = thymine
  modified_base 689: mod_base = OTHER; note = thymine
  modified_base 691..692: mod_base = OTHER; note = thymine
  modified_base 694..698: mod_base = OTHER; note = thymine
  modified_base 702: mod_base = OTHER; note = thymine
  modified_base 710: mod_base = OTHER; note = thymine
  modified_base 713..714: mod_base = OTHER; note = thymine
  modified_base 718..719: mod_base = OTHER; note = thymine
  modified_base 722: mod_base = OTHER; note = thymine
  modified_base 727..729: mod_base = OTHER; note = thymine
  modified_base 736: mod_base = OTHER; note = thymine
  modified_base 738: mod_base = OTHER; note = thymine
  modified_base 742: mod_base = OTHER; note = thymine
  modified_base 744: mod_base = OTHER; note = thymine
  modified_base 747: mod_base = OTHER; note = thymine
  modified_base 749: mod_base = OTHER; note = thymine
  modified_base 752: mod_base = OTHER; note = thymine
  modified_base 755: mod_base = OTHER; note = thymine
  modified_base 760..761: mod_base = OTHER; note = thymine
  modified_base 765..766: mod_base = OTHER; note = thymine
  modified_base 770: mod_base = OTHER; note = thymine
  modified_base 772: mod_base = OTHER; note = thymine
  modified_base 777: mod_base = OTHER; note = thymine
  modified_base 789: mod_base = OTHER; note = thymine
  modified_base 794: mod_base = OTHER; note = thymine
  modified_base 796: mod_base = OTHER; note = thymine
  modified_base 798: mod_base = OTHER; note = thymine
  modified_base 800: mod_base = OTHER; note = thymine
  modified_base 802..803: mod_base = OTHER; note = thymine
  modified_base 811..813: mod_base = OTHER; note = thymine
  modified_base 816..817: mod_base = OTHER; note = thymine
  modified_base 821: mod_base = OTHER; note = thymine
  modified_base 823: mod_base = OTHER; note = thymine
  modified_base 825: mod_base = OTHER; note = thymine
  modified_base 832: mod_base = OTHER; note = thymine
  modified_base 837: mod_base = OTHER; note = thymine
  modified_base 839..841: mod_base = OTHER; note = thymine
  modified_base 854..855: mod_base = OTHER; note = thymine
  modified_base 857: mod_base = OTHER; note = thymine
  modified_base 860: mod_base = OTHER; note = thymine
  modified_base 864..865: mod_base = OTHER; note = thymine
  modified_base 867: mod_base = OTHER; note = thymine
  modified_base 869: mod_base = OTHER; note = thymine
  modified_base 877: mod_base = OTHER; note = thymine
  modified_base 882: mod_base = OTHER; note = thymine
  modified_base 885: mod_base = OTHER; note = thymine
  modified_base 887: mod_base = OTHER; note = thymine
  modified_base 891: mod_base = OTHER; note = thymine
  modified_base 894..895: mod_base = OTHER; note = thymine
  modified_base 897..898: mod_base = OTHER; note = thymine
  modified_base 901..902: mod_base = OTHER; note = thymine
  modified_base 905..906: mod_base = OTHER; note = thymine
  modified_base 912: mod_base = OTHER; note = thymine
  modified_base 918: mod_base = OTHER; note = thymine
  modified_base 927..928: mod_base = OTHER; note = thymine
  modified_base 933..934: mod_base = OTHER; note = thymine
  modified_base 939: mod_base = OTHER; note = thymine
  modified_base 943: mod_base = OTHER; note = thymine
  modified_base 949: mod_base = OTHER; note = thymine
  modified_base 951..952: mod_base = OTHER; note = thymine
  modified_base 954: mod_base = OTHER; note = thymine
  modified_base 957: mod_base = OTHER; note = thymine
  modified_base 960..962: mod_base = OTHER; note = thymine
  modified_base 964: mod_base = OTHER; note = thymine
  modified_base 972: mod_base = OTHER; note = thymine
  modified_base 977: mod_base = OTHER; note = thymine
  modified_base 981: mod_base = OTHER; note = thymine
  modified_base 985: mod_base = OTHER; note = thymine
  modified_base 998: mod_base = OTHER; note = thymine
  modified_base 1002: mod_base = OTHER; note = thymine
  modified_base 1009: mod_base = OTHER; note = thymine
  modified_base 1011: mod_base = OTHER; note = thymine
  modified_base 1014: mod_base = OTHER; note = thymine
  modified_base 1019: mod_base = OTHER; note = thymine
  modified_base 1021: mod_base = OTHER; note = thymine
  modified_base 1028: mod_base = OTHER; note = thymine
  modified_base 1031: mod_base = OTHER; note = thymine
  modified_base 1036: mod_base = OTHER; note = thymine
  modified_base 1042: mod_base = OTHER; note = thymine
  modified_base 1049: mod_base = OTHER; note = thymine
  modified_base 1056: mod_base = OTHER; note = thymine
  modified_base 1064: mod_base = OTHER; note = thymine
  modified_base 1068: mod_base = OTHER; note = thymine
  modified_base 1072..1073: mod_base = OTHER; note = thymine
  modified_base 1077: mod_base = OTHER; note = thymine
  modified_base 1094..1095: mod_base = OTHER; note = thymine
  modified_base 1099: mod_base = OTHER; note = thymine
  modified_base 1102..1103: mod_base = OTHER; note = thymine
  modified_base 1107: mod_base = OTHER; note = thymine
  modified_base 1113..1114: mod_base = OTHER; note = thymine
  modified_base 1119: mod_base = OTHER; note = thymine
  modified_base 1121: mod_base = OTHER; note = thymine
  modified_base 1124..1125: mod_base = OTHER; note = thymine
  modified_base 1132: mod_base = OTHER; note = thymine
  modified_base 1140: mod_base = OTHER; note = thymine
  modified_base 1143: mod_base = OTHER; note = thymine
  modified_base 1148: mod_base = OTHER; note = thymine
  modified_base 1151: mod_base = OTHER; note = thymine
  modified_base 1154: mod_base = OTHER; note = thymine
  modified_base 1159: mod_base = OTHER; note = thymine
  modified_base 1165: mod_base = OTHER; note = thymine
  modified_base 1168: mod_base = OTHER; note = thymine
  modified_base 1170: mod_base = OTHER; note = thymine
  modified_base 1174: mod_base = OTHER; note = thymine
  modified_base 1176: mod_base = OTHER; note = thymine
  modified_base 1183: mod_base = OTHER; note = thymine
  modified_base 1185..1187: mod_base = OTHER; note = thymine
  modified_base 1189..1190: mod_base = OTHER; note = thymine
  modified_base 1192: mod_base = OTHER; note = thymine
  modified_base 1196..1198: mod_base = OTHER; note = thymine
  modified_base 1202..1205: mod_base = OTHER; note = thymine
  modified_base 1207: mod_base = OTHER; note = thymine
  modified_base 1214: mod_base = OTHER; note = thymine
  modified_base 1217: mod_base = OTHER; note = thymine
  modified_base 1219: mod_base = OTHER; note = thymine
  modified_base 1221: mod_base = OTHER; note = thymine
  modified_base 1223..1224: mod_base = OTHER; note = thymine
  modified_base 1226: mod_base = OTHER; note = thymine
  modified_base 1229..1230: mod_base = OTHER; note = thymine
  modified_base 1234: mod_base = OTHER; note = thymine
  modified_base 1237: mod_base = OTHER; note = thymine
  modified_base 1241..1244: mod_base = OTHER; note = thymine
  modified_base 1247..1249: mod_base = OTHER; note = thymine
  modified_base 1256..1257: mod_base = OTHER; note = thymine
  modified_base 1259..1260: mod_base = OTHER; note = thymine
  modified_base 1272..1273: mod_base = OTHER; note = thymine
  modified_base 1285: mod_base = OTHER; note = thymine
  modified_base 1287: mod_base = OTHER; note = thymine
  modified_base 1293: mod_base = OTHER; note = thymine
  modified_base 1297: mod_base = OTHER; note = thymine
  modified_base 1300: mod_base = OTHER; note = thymine
  modified_base 1302: mod_base = OTHER; note = thymine
  modified_base 1304..1305: mod_base = OTHER; note = thymine
  modified_base 1308: mod_base = OTHER; note = thymine
  modified_base 1324: mod_base = OTHER; note = thymine
  modified_base 1326..1327: mod_base = OTHER; note = thymine
  modified_base 1329: mod_base = OTHER; note = thymine
  modified_base 1344: mod_base = OTHER; note = thymine
  modified_base 1356..1357: mod_base = OTHER; note = thymine
  modified_base 1360: mod_base = OTHER; note = thymine
  modified_base 1362..1363: mod_base = OTHER; note = thymine
  modified_base 1365: mod_base = OTHER; note = thymine
  modified_base 1372: mod_base = OTHER; note = thymine
  modified_base 1379: mod_base = OTHER; note = thymine
  modified_base 1383: mod_base = OTHER; note = thymine
  modified_base 1390..1391: mod_base = OTHER; note = thymine
  modified_base 1396: mod_base = OTHER; note = thymine
  modified_base 1398: mod_base = OTHER; note = thymine
  modified_base 1400..1401: mod_base = OTHER; note = thymine
  modified_base 1411..1412: mod_base = OTHER; note = thymine
  modified_base 1415: mod_base = OTHER; note = thymine
  modified_base 1417..1418: mod_base = OTHER; note = thymine
  modified_base 1421: mod_base = OTHER; note = thymine
  modified_base 1424..1426: mod_base = OTHER; note = thymine
  modified_base 1428..1429: mod_base = OTHER; note = thymine
  modified_base 1431..1432: mod_base = OTHER; note = thymine
  modified_base 1435: mod_base = OTHER; note = thymine
  modified_base 1439: mod_base = OTHER; note = thymine
  modified_base 1447: mod_base = OTHER; note = thymine
  modified_base 1452: mod_base = OTHER; note = thymine
  modified_base 1454: mod_base = OTHER; note = thymine
  modified_base 1456: mod_base = OTHER; note = thymine
  modified_base 1460..1461: mod_base = OTHER; note = thymine
  modified_base 1466: mod_base = OTHER; note = thymine
  modified_base 1470: mod_base = OTHER; note = thymine
  modified_base 1472: mod_base = OTHER; note = thymine
  modified_base 1475..1476: mod_base = OTHER; note = thymine
  modified_base 1480: mod_base = OTHER; note = thymine
  modified_base 1482: mod_base = OTHER; note = thymine
  modified_base 1485: mod_base = OTHER; note = thymine
  modified_base 1487..1488: mod_base = OTHER; note = thymine
  modified_base 1490: mod_base = OTHER; note = thymine
  modified_base 1494: mod_base = OTHER; note = thymine
  modified_base 1496: mod_base = OTHER; note = thymine
  modified_base 1502..1503: mod_base = OTHER; note = thymine
  modified_base 1506..1507: mod_base = OTHER; note = thymine
  modified_base 1523..1525: mod_base = OTHER; note = thymine
  modified_base 1535: mod_base = OTHER; note = thymine
  modified_base 1537..1538: mod_base = OTHER; note = thymine
  modified_base 1543: mod_base = OTHER; note = thymine
  modified_base 1547: mod_base = OTHER; note = thymine
  modified_base 1550: mod_base = OTHER; note = thymine
  modified_base 1554..1556: mod_base = OTHER; note = thymine
  modified_base 1558: mod_base = OTHER; note = thymine
  modified_base 1561: mod_base = OTHER; note = thymine
  modified_base 1568: mod_base = OTHER; note = thymine
  modified_base 1577..1578: mod_base = OTHER; note = thymine
  modified_base 1585: mod_base = OTHER; note = thymine
  modified_base 1590..1591: mod_base = OTHER; note = thymine
  modified_base 1594: mod_base = OTHER; note = thymine
  modified_base 1601..1602: mod_base = OTHER; note = thymine
  modified_base 1605: mod_base = OTHER; note = thymine
  modified_base 1607: mod_base = OTHER; note = thymine
  modified_base 1612: mod_base = OTHER; note = thymine
  modified_base 1615: mod_base = OTHER; note = thymine
  modified_base 1622..1623: mod_base = OTHER; note = thymine
  modified_base 1629..1631: mod_base = OTHER; note = thymine
  modified_base 1633: mod_base = OTHER; note = thymine
  modified_base 1635: mod_base = OTHER; note = thymine
  modified_base 1638: mod_base = OTHER; note = thymine
  modified_base 1640: mod_base = OTHER; note = thymine
  modified_base 1644: mod_base = OTHER; note = thymine
  modified_base 1646: mod_base = OTHER; note = thymine
  modified_base 1648: mod_base = OTHER; note = thymine
  modified_base 1653: mod_base = OTHER; note = thymine
  modified_base 1658: mod_base = OTHER; note = thymine
  modified_base 1668: mod_base = OTHER; note = thymine
  modified_base 1672: mod_base = OTHER; note = thymine
  modified_base 1674: mod_base = OTHER; note = thymine
  modified_base 1676..1678: mod_base = OTHER; note = thymine
  modified_base 1684: mod_base = OTHER; note = thymine
  modified_base 1686: mod_base = OTHER; note = thymine
  modified_base 1692..1693: mod_base = OTHER; note = thymine
  modified_base 1695..1696: mod_base = OTHER; note = thymine
  modified_base 1698: mod_base = OTHER; note = thymine
  modified_base 1700: mod_base = OTHER; note = thymine
  modified_base 1702..1703: mod_base = OTHER; note = thymine
  modified_base 1706..1708: mod_base = OTHER; note = thymine
  modified_base 1712: mod_base = OTHER; note = thymine
  modified_base 1716: mod_base = OTHER; note = thymine
  modified_base 1720..1721: mod_base = OTHER; note = thymine
  modified_base 1723: mod_base = OTHER; note = thymine
  modified_base 1726: mod_base = OTHER; note = thymine
  modified_base 1730: mod_base = OTHER; note = thymine
  modified_base 1734..1737: mod_base = OTHER; note = thymine
  modified_base 1740..1741: mod_base = OTHER; note = thymine
  modified_base 1744: mod_base = OTHER; note = thymine
  modified_base 1747: mod_base = OTHER; note = thymine
  modified_base 1749: mod_base = OTHER; note = thymine
  modified_base 1751: mod_base = OTHER; note = thymine
  modified_base 1767: mod_base = OTHER; note = thymine
  modified_base 1769: mod_base = OTHER; note = thymine
  modified_base 1776..1777: mod_base = OTHER; note = thymine
  modified_base 1781: mod_base = OTHER; note = thymine
  modified_base 1783..1784: mod_base = OTHER; note = thymine
  modified_base 1789: mod_base = OTHER; note = thymine
  modified_base 1791: mod_base = OTHER; note = thymine
  modified_base 1799: mod_base = OTHER; note = thymine
  modified_base 1801: mod_base = OTHER; note = thymine
  modified_base 1803..1804: mod_base = OTHER; note = thymine
  modified_base 1808..1809: mod_base = OTHER; note = thymine
  modified_base 1815: mod_base = OTHER; note = thymine
  modified_base 1818..1819: mod_base = OTHER; note = thymine
  modified_base 1822: mod_base = OTHER; note = thymine
  modified_base 1829: mod_base = OTHER; note = thymine
  modified_base 1836: mod_base = OTHER; note = thymine
  modified_base 1838..1839: mod_base = OTHER; note = thymine
  modified_base 1841..1842: mod_base = OTHER; note = thymine
  modified_base 1847: mod_base = OTHER; note = thymine
  modified_base 1850..1852: mod_base = OTHER; note = thymine
  modified_base 1857: mod_base = OTHER; note = thymine
  modified_base 1860..1862: mod_base = OTHER; note = thymine
  modified_base 1864..1865: mod_base = OTHER; note = thymine
  modified_base 1868: mod_base = OTHER; note = thymine
  modified_base 1870: mod_base = OTHER; note = thymine
  modified_base 1875: mod_base = OTHER; note = thymine
  modified_base 1880: mod_base = OTHER; note = thymine
  modified_base 1884: mod_base = OTHER; note = thymine
  modified_base 1887..1888: mod_base = OTHER; note = thymine
  modified_base 1891: mod_base = OTHER; note = thymine
  modified_base 1895: mod_base = OTHER; note = thymine
  modified_base 1898..1899: mod_base = OTHER; note = thymine
  modified_base 1902: mod_base = OTHER; note = thymine
  modified_base 1904..1905: mod_base = OTHER; note = thymine
  modified_base 1907: mod_base = OTHER; note = thymine
  modified_base 1909: mod_base = OTHER; note = thymine
  modified_base 1911: mod_base = OTHER; note = thymine
  modified_base 1913: mod_base = OTHER; note = thymine
  modified_base 1916: mod_base = OTHER; note = thymine
  modified_base 1919: mod_base = OTHER; note = thymine
  modified_base 1922..1924: mod_base = OTHER; note = thymine
  modified_base 1928..1930: mod_base = OTHER; note = thymine
  modified_base 1947..1948: mod_base = OTHER; note = thymine
  modified_base 1950..1951: mod_base = OTHER; note = thymine
  modified_base 1953..1954: mod_base = OTHER; note = thymine
  modified_base 1958: mod_base = OTHER; note = thymine
  modified_base 1974: mod_base = OTHER; note = thymine
  modified_base 1976: mod_base = OTHER; note = thymine
  modified_base 1981: mod_base = OTHER; note = thymine
  modified_base 1983..1984: mod_base = OTHER; note = thymine
  modified_base 1987: mod_base = OTHER; note = thymine
  modified_base 2004: mod_base = OTHER; note = thymine
  modified_base 2009: mod_base = OTHER; note = thymine
  modified_base 2013: mod_base = OTHER; note = thymine
  modified_base 2022..2024: mod_base = OTHER; note = thymine
  modified_base 2026: mod_base = OTHER; note = thymine
  modified_base 2043: mod_base = OTHER; note = thymine
  modified_base 2046: mod_base = OTHER; note = thymine
  modified_base 2056..2057: mod_base = OTHER; note = thymine
  modified_base 2060..2062: mod_base = OTHER; note = thymine
  modified_base 2069..2070: mod_base = OTHER; note = thymine
  modified_base 2073..2074: mod_base = OTHER; note = thymine
  modified_base 2078..2079: mod_base = OTHER; note = thymine
  modified_base 2081..2083: mod_base = OTHER; note = thymine
  modified_base 2087: mod_base = OTHER; note = thymine
  modified_base 2090..2094: mod_base = OTHER; note = thymine
  modified_base 2100: mod_base = OTHER; note = thymine
  modified_base 2104: mod_base = OTHER; note = thymine
  modified_base 2108..2110: mod_base = OTHER; note = thymine
  modified_base 2115..2117: mod_base = OTHER; note = thymine
  modified_base 2119: mod_base = OTHER; note = thymine
  modified_base 2121: mod_base = OTHER; note = thymine
  modified_base 2128..2129: mod_base = OTHER; note = thymine
  modified_base 2141: mod_base = OTHER; note = thymine
  modified_base 2146..2147: mod_base = OTHER; note = thymine
  modified_base 2149..2150: mod_base = OTHER; note = thymine
  modified_base 2155: mod_base = OTHER; note = thymine
  modified_base 2159: mod_base = OTHER; note = thymine
  modified_base 2161: mod_base = OTHER; note = thymine
  modified_base 2163: mod_base = OTHER; note = thymine
  modified_base 2166..2168: mod_base = OTHER; note = thymine
  modified_base 2170: mod_base = OTHER; note = thymine
  modified_base 2172: mod_base = OTHER; note = thymine
  modified_base 2180: mod_base = OTHER; note = thymine
  modified_base 2186..2188: mod_base = OTHER; note = thymine
  modified_base 2193: mod_base = OTHER; note = thymine
  modified_base 2197: mod_base = OTHER; note = thymine
  modified_base 2205..2206: mod_base = OTHER; note = thymine
  modified_base 2209: mod_base = OTHER; note = thymine
  modified_base 2215: mod_base = OTHER; note = thymine
  modified_base 2218: mod_base = OTHER; note = thymine
  modified_base 2220..2221: mod_base = OTHER; note = thymine
  modified_base 2233: mod_base = OTHER; note = thymine
  modified_base 2240: mod_base = OTHER; note = thymine
  modified_base 2256: mod_base = OTHER; note = thymine
  modified_base 2261: mod_base = OTHER; note = thymine
  modified_base 2267..2269: mod_base = OTHER; note = thymine
  modified_base 2271..2272: mod_base = OTHER; note = thymine
  modified_base 2275..2276: mod_base = OTHER; note = thymine
  modified_base 2280: mod_base = OTHER; note = thymine
  modified_base 2282: mod_base = OTHER; note = thymine
  modified_base 2290: mod_base = OTHER; note = thymine
  modified_base 2292..2293: mod_base = OTHER; note = thymine
  modified_base 2295..2296: mod_base = OTHER; note = thymine
  modified_base 2303..2306: mod_base = OTHER; note = thymine
  modified_base 2308: mod_base = OTHER; note = thymine
  modified_base 2313..2315: mod_base = OTHER; note = thymine
  modified_base 2317..2318: mod_base = OTHER; note = thymine
  modified_base 2326: mod_base = OTHER; note = thymine
  modified_base 2331: mod_base = OTHER; note = thymine
  modified_base 2333: mod_base = OTHER; note = thymine
  modified_base 2335..2336: mod_base = OTHER; note = thymine
  modified_base 2338: mod_base = OTHER; note = thymine
  modified_base 2341..2342: mod_base = OTHER; note = thymine
  modified_base 2344: mod_base = OTHER; note = thymine
  modified_base 2348: mod_base = OTHER; note = thymine
  modified_base 2350..2351: mod_base = OTHER; note = thymine
  modified_base 2356: mod_base = OTHER; note = thymine
  modified_base 2358: mod_base = OTHER; note = thymine
  modified_base 2360..2361: mod_base = OTHER; note = thymine
  modified_base 2363..2365: mod_base = OTHER; note = thymine
  modified_base 2367: mod_base = OTHER; note = thymine
  modified_base 2369: mod_base = OTHER; note = thymine
  modified_base 2375: mod_base = OTHER; note = thymine
  modified_base 2377..2378: mod_base = OTHER; note = thymine
  modified_base 2383..2384: mod_base = OTHER; note = thymine
  modified_base 2386: mod_base = OTHER; note = thymine
  modified_base 2389: mod_base = OTHER; note = thymine
  modified_base 2393..2395: mod_base = OTHER; note = thymine
  modified_base 2398: mod_base = OTHER; note = thymine
  modified_base 2401: mod_base = OTHER; note = thymine
  modified_base 2413: mod_base = OTHER; note = thymine
  modified_base 2418: mod_base = OTHER; note = thymine
  modified_base 2428: mod_base = OTHER; note = thymine
  modified_base 2432: mod_base = OTHER; note = thymine
  modified_base 2436..2437: mod_base = OTHER; note = thymine
  modified_base 2444: mod_base = OTHER; note = thymine
  modified_base 2446: mod_base = OTHER; note = thymine
  modified_base 2448: mod_base = OTHER; note = thymine
  modified_base 2451: mod_base = OTHER; note = thymine
  modified_base 2453: mod_base = OTHER; note = thymine
  modified_base 2458..2460: mod_base = OTHER; note = thymine
  modified_base 2462: mod_base = OTHER; note = thymine
  modified_base 2464: mod_base = OTHER; note = thymine
  modified_base 2467..2469: mod_base = OTHER; note = thymine
  modified_base 2472: mod_base = OTHER; note = thymine
  modified_base 2474..2475: mod_base = OTHER; note = thymine
  modified_base 2479: mod_base = OTHER; note = thymine
  modified_base 2490: mod_base = OTHER; note = thymine
  modified_base 2492..2493: mod_base = OTHER; note = thymine
  modified_base 2501: mod_base = OTHER; note = thymine
  modified_base 2507: mod_base = OTHER; note = thymine
  modified_base 2514: mod_base = OTHER; note = thymine
  modified_base 2517: mod_base = OTHER; note = thymine
  modified_base 2522: mod_base = OTHER; note = thymine
  modified_base 2527: mod_base = OTHER; note = thymine
  modified_base 2530..2531: mod_base = OTHER; note = thymine
  modified_base 2533: mod_base = OTHER; note = thymine
  modified_base 2538..2539: mod_base = OTHER; note = thymine
  modified_base 2542: mod_base = OTHER; note = thymine
  modified_base 2547..2550: mod_base = OTHER; note = thymine
  modified_base 2553: mod_base = OTHER; note = thymine
  modified_base 2560..2563: mod_base = OTHER; note = thymine
  modified_base 2567: mod_base = OTHER; note = thymine
  modified_base 2569: mod_base = OTHER; note = thymine
  modified_base 2571..2572: mod_base = OTHER; note = thymine
  modified_base 2575: mod_base = OTHER; note = thymine
  modified_base 2577: mod_base = OTHER; note = thymine
  modified_base 2580..2581: mod_base = OTHER; note = thymine
  modified_base 2588..2590: mod_base = OTHER; note = thymine
  modified_base 2593: mod_base = OTHER; note = thymine
  modified_base 2601..2602: mod_base = OTHER; note = thymine
  modified_base 2605..2606: mod_base = OTHER; note = thymine
  modified_base 2608: mod_base = OTHER; note = thymine
  modified_base 2614..2615: mod_base = OTHER; note = thymine
  modified_base 2617: mod_base = OTHER; note = thymine
  modified_base 2619: mod_base = OTHER; note = thymine
  modified_base 2622..2623: mod_base = OTHER; note = thymine
  modified_base 2625: mod_base = OTHER; note = thymine
  modified_base 2628: mod_base = OTHER; note = thymine
  modified_base 2638: mod_base = OTHER; note = thymine
  modified_base 2642..2643: mod_base = OTHER; note = thymine
  modified_base 2647..2650: mod_base = OTHER; note = thymine
  modified_base 2653: mod_base = OTHER; note = thymine
  modified_base 2655: mod_base = OTHER; note = thymine
  modified_base 2657: mod_base = OTHER; note = thymine
  modified_base 2664: mod_base = OTHER; note = thymine
  modified_base 2669: mod_base = OTHER; note = thymine
  modified_base 2671: mod_base = OTHER; note = thymine
  modified_base 2679: mod_base = OTHER; note = thymine
  modified_base 2688: mod_base = OTHER; note = thymine
  modified_base 2690..2692: mod_base = OTHER; note = thymine
  modified_base 2697: mod_base = OTHER; note = thymine
  modified_base 2699..2700: mod_base = OTHER; note = thymine
  modified_base 2702..2703: mod_base = OTHER; note = thymine
  modified_base 2705..2706: mod_base = OTHER; note = thymine
  modified_base 2710: mod_base = OTHER; note = thymine
  modified_base 2714: mod_base = OTHER; note = thymine
  modified_base 2725: mod_base = OTHER; note = thymine
  modified_base 2727: mod_base = OTHER; note = thymine
  modified_base 2729: mod_base = OTHER; note = thymine
  modified_base 2731: mod_base = OTHER; note = thymine
  modified_base 2733: mod_base = OTHER; note = thymine
  modified_base 2735: mod_base = OTHER; note = thymine
  modified_base 2742: mod_base = OTHER; note = thymine
  modified_base 2744: mod_base = OTHER; note = thymine
  modified_base 2746: mod_base = OTHER; note = thymine
  modified_base 2749: mod_base = OTHER; note = thymine
  modified_base 2753..2754: mod_base = OTHER; note = thymine
  modified_base 2757..2758: mod_base = OTHER; note = thymine
  modified_base 2762: mod_base = OTHER; note = thymine
  modified_base 2764: mod_base = OTHER; note = thymine
  modified_base 2767: mod_base = OTHER; note = thymine
  modified_base 2773: mod_base = OTHER; note = thymine
  modified_base 2775..2777: mod_base = OTHER; note = thymine
  modified_base 2783: mod_base = OTHER; note = thymine
  modified_base 2785: mod_base = OTHER; note = thymine
  modified_base 2787: mod_base = OTHER; note = thymine
  modified_base 2791: mod_base = OTHER; note = thymine
  modified_base 2794: mod_base = OTHER; note = thymine
  modified_base 2802..2803: mod_base = OTHER; note = thymine
  modified_base 2809: mod_base = OTHER; note = thymine
  modified_base 2813..2814: mod_base = OTHER; note = thymine
  modified_base 2817: mod_base = OTHER; note = thymine
  modified_base 2824..2825: mod_base = OTHER; note = thymine
  modified_base 2827: mod_base = OTHER; note = thymine
  modified_base 2829: mod_base = OTHER; note = thymine
  modified_base 2831: mod_base = OTHER; note = thymine
  modified_base 2833: mod_base = OTHER; note = thymine
  modified_base 2836: mod_base = OTHER; note = thymine
  modified_base 2838: mod_base = OTHER; note = thymine
  modified_base 2840: mod_base = OTHER; note = thymine
  modified_base 2846..2847: mod_base = OTHER; note = thymine
  modified_base 2849: mod_base = OTHER; note = thymine
  modified_base 2851..2852: mod_base = OTHER; note = thymine
  modified_base 2857..2860: mod_base = OTHER; note = thymine
  modified_base 2865..2866: mod_base = OTHER; note = thymine
  modified_base 2871..2872: mod_base = OTHER; note = thymine
  modified_base 2874: mod_base = OTHER; note = thymine
  modified_base 2876: mod_base = OTHER; note = thymine
  modified_base 2895..2896: mod_base = OTHER; note = thymine
  modified_base 2899: mod_base = OTHER; note = thymine
  modified_base 2903: mod_base = OTHER; note = thymine
  modified_base 2911: mod_base = OTHER; note = thymine
  modified_base 2913: mod_base = OTHER; note = thymine
  modified_base 2915..2916: mod_base = OTHER; note = thymine
  modified_base 2923: mod_base = OTHER; note = thymine
  modified_base 2933: mod_base = OTHER; note = thymine
  modified_base 2935..2936: mod_base = OTHER; note = thymine
  modified_base 2940..2941: mod_base = OTHER; note = thymine
  modified_base 2946: mod_base = OTHER; note = thymine
  modified_base 2959..2960: mod_base = OTHER; note = thymine
  modified_base 2963: mod_base = OTHER; note = thymine
  modified_base 2965: mod_base = OTHER; note = thymine
  modified_base 2971: mod_base = OTHER; note = thymine
  modified_base 2973..2975: mod_base = OTHER; note = thymine
  modified_base 2980: mod_base = OTHER; note = thymine
  modified_base 2983: mod_base = OTHER; note = thymine
  modified_base 2988..2989: mod_base = OTHER; note = thymine
  modified_base 2991..2993: mod_base = OTHER; note = thymine
  modified_base 2996..2998: mod_base = OTHER; note = thymine
  modified_base 3005: mod_base = OTHER; note = thymine
  modified_base 3011..3012: mod_base = OTHER; note = thymine
  modified_base 3015: mod_base = OTHER; note = thymine
  modified_base 3028..3030: mod_base = OTHER; note = thymine
  modified_base 3038..3039: mod_base = OTHER; note = thymine
  modified_base 3041: mod_base = OTHER; note = thymine
  modified_base 3043: mod_base = OTHER; note = thymine
  modified_base 3046: mod_base = OTHER; note = thymine
  modified_base 3048: mod_base = OTHER; note = thymine
  modified_base 3050..3051: mod_base = OTHER; note = thymine
  modified_base 3057..3060: mod_base = OTHER; note = thymine
  modified_base 3063: mod_base = OTHER; note = thymine
  modified_base 3067: mod_base = OTHER; note = thymine
  modified_base 3079..3083: mod_base = OTHER; note = thymine
  modified_base 3085: mod_base = OTHER; note = thymine
  modified_base 3091: mod_base = OTHER; note = thymine
  modified_base 3096: mod_base = OTHER; note = thymine
  modified_base 3099: mod_base = OTHER; note = thymine
  modified_base 3102: mod_base = OTHER; note = thymine
  modified_base 3106: mod_base = OTHER; note = thymine
  modified_base 3112: mod_base = OTHER; note = thymine
  modified_base 3114: mod_base = OTHER; note = thymine
  modified_base 3118: mod_base = OTHER; note = thymine
  modified_base 3125: mod_base = OTHER; note = thymine
  modified_base 3127: mod_base = OTHER; note = thymine
  modified_base 3132: mod_base = OTHER; note = thymine
  modified_base 3135: mod_base = OTHER; note = thymine
  modified_base 3140: mod_base = OTHER; note = thymine
  modified_base 3144: mod_base = OTHER; note = thymine
  modified_base 3149: mod_base = OTHER; note = thymine
  modified_base 3153: mod_base = OTHER; note = thymine
  modified_base 3164..3165: mod_base = OTHER; note = thymine
  modified_base 3167..3168: mod_base = OTHER; note = thymine
  modified_base 3172: mod_base = OTHER; note = thymine
  modified_base 3175..3176: mod_base = OTHER; note = thymine
  modified_base 3183: mod_base = OTHER; note = thymine
  modified_base 3185: mod_base = OTHER; note = thymine
  modified_base 3189: mod_base = OTHER; note = thymine
  modified_base 3191: mod_base = OTHER; note = thymine
  modified_base 3194: mod_base = OTHER; note = thymine
  modified_base 3196: mod_base = OTHER; note = thymine
  modified_base 3198..3199: mod_base = OTHER; note = thymine
  modified_base 3201: mod_base = OTHER; note = thymine
  modified_base 3208..3209: mod_base = OTHER; note = thymine
  modified_base 3213: mod_base = OTHER; note = thymine
  modified_base 3220: mod_base = OTHER; note = thymine
  modified_base 3222: mod_base = OTHER; note = thymine
  modified_base 3235..3236: mod_base = OTHER; note = thymine
  modified_base 3238: mod_base = OTHER; note = thymine
  modified_base 3240: mod_base = OTHER; note = thymine
  modified_base 3244: mod_base = OTHER; note = thymine
  modified_base 3252: mod_base = OTHER; note = thymine
  modified_base 3255: mod_base = OTHER; note = thymine
  modified_base 3259..3260: mod_base = OTHER; note = thymine
  modified_base 3264: mod_base = OTHER; note = thymine
  modified_base 3270: mod_base = OTHER; note = thymine
  modified_base 3273: mod_base = OTHER; note = thymine
  modified_base 3276: mod_base = OTHER; note = thymine
  modified_base 3285: mod_base = OTHER; note = thymine
  modified_base 3287..3288: mod_base = OTHER; note = thymine
  modified_base 3298: mod_base = OTHER; note = thymine
  modified_base 3301: mod_base = OTHER; note = thymine
  modified_base 3306: mod_base = OTHER; note = thymine
  modified_base 3316..3317: mod_base = OTHER; note = thymine
  modified_base 3323: mod_base = OTHER; note = thymine
  modified_base 3329: mod_base = OTHER; note = thymine
  modified_base 3331: mod_base = OTHER; note = thymine
  modified_base 3333..3334: mod_base = OTHER; note = thymine
  modified_base 3338..3340: mod_base = OTHER; note = thymine
  modified_base 3344: mod_base = OTHER; note = thymine
  modified_base 3351: mod_base = OTHER; note = thymine
  modified_base 3356..3357: mod_base = OTHER; note = thymine
  modified_base 3359: mod_base = OTHER; note = thymine
  modified_base 3366..3367: mod_base = OTHER; note = thymine
  modified_base 3370..3371: mod_base = OTHER; note = thymine
  modified_base 3377..3378: mod_base = OTHER; note = thymine
  modified_base 3387..3388: mod_base = OTHER; note = thymine
  modified_base 3390: mod_base = OTHER; note = thymine
  modified_base 3396: mod_base = OTHER; note = thymine
  modified_base 3401..3403: mod_base = OTHER; note = thymine
  modified_base 3409: mod_base = OTHER; note = thymine
  modified_base 3411..3412: mod_base = OTHER; note = thymine
  modified_base 3415..3416: mod_base = OTHER; note = thymine
  modified_base 3419: mod_base = OTHER; note = thymine
  modified_base 3422..3423: mod_base = OTHER; note = thymine
  modified_base 3429..3430: mod_base = OTHER; note = thymine
  modified_base 3435..3437: mod_base = OTHER; note = thymine
  modified_base 3439..3440: mod_base = OTHER; note = thymine
  modified_base 3442: mod_base = OTHER; note = thymine
  modified_base 3444: mod_base = OTHER; note = thymine
  modified_base 3446: mod_base = OTHER; note = thymine
  modified_base 3448: mod_base = OTHER; note = thymine
  modified_base 3453..3454: mod_base = OTHER; note = thymine
  modified_base 3456..3457: mod_base = OTHER; note = thymine
  modified_base 3461..3462: mod_base = OTHER; note = thymine
  modified_base 3464: mod_base = OTHER; note = thymine
  modified_base 3467: mod_base = OTHER; note = thymine
  modified_base 3469: mod_base = OTHER; note = thymine
  modified_base 3471: mod_base = OTHER; note = thymine
  modified_base 3491: mod_base = OTHER; note = thymine
  modified_base 3494: mod_base = OTHER; note = thymine
  modified_base 3499: mod_base = OTHER; note = thymine
  modified_base 3502: mod_base = OTHER; note = thymine
  modified_base 3505..3506: mod_base = OTHER; note = thymine
  modified_base 3509: mod_base = OTHER; note = thymine
  modified_base 3515..3517: mod_base = OTHER; note = thymine
  modified_base 3522: mod_base = OTHER; note = thymine
  modified_base 3525: mod_base = OTHER; note = thymine
  modified_base 3528: mod_base = OTHER; note = thymine
  modified_base 3530: mod_base = OTHER; note = thymine
  modified_base 3543: mod_base = OTHER; note = thymine
  modified_base 3549..3550: mod_base = OTHER; note = thymine
  modified_base 3552..3553: mod_base = OTHER; note = thymine
  modified_base 3562..3565: mod_base = OTHER; note = thymine
  modified_base 3568: mod_base = OTHER; note = thymine
  modified_base 3570: mod_base = OTHER; note = thymine
  modified_base 3577..3578: mod_base = OTHER; note = thymine
  modified_base 3584..3585: mod_base = OTHER; note = thymine
  modified_base 3590..3591: mod_base = OTHER; note = thymine
  modified_base 3594..3597: mod_base = OTHER; note = thymine
  modified_base 3599: mod_base = OTHER; note = thymine
  modified_base 3603..3604: mod_base = OTHER; note = thymine
  modified_base 3611: mod_base = OTHER; note = thymine
  modified_base 3613: mod_base = OTHER; note = thymine
  modified_base 3615: mod_base = OTHER; note = thymine
  modified_base 3617..3619: mod_base = OTHER; note = thymine
  modified_base 3622..3623: mod_base = OTHER; note = thymine
  modified_base 3628: mod_base = OTHER; note = thymine
  modified_base 3638..3639: mod_base = OTHER; note = thymine
  modified_base 3641: mod_base = OTHER; note = thymine
  modified_base 3648: mod_base = OTHER; note = thymine
  modified_base 3650..3651: mod_base = OTHER; note = thymine
  modified_base 3656: mod_base = OTHER; note = thymine
  modified_base 3661: mod_base = OTHER; note = thymine
  modified_base 3664: mod_base = OTHER; note = thymine
  modified_base 3667: mod_base = OTHER; note = thymine
  modified_base 3672..3673: mod_base = OTHER; note = thymine
  modified_base 3676: mod_base = OTHER; note = thymine
  modified_base 3678..3679: mod_base = OTHER; note = thymine
  modified_base 3684: mod_base = OTHER; note = thymine
  modified_base 3686: mod_base = OTHER; note = thymine
  modified_base 3693: mod_base = OTHER; note = thymine
  modified_base 3698..3699: mod_base = OTHER; note = thymine
  modified_base 3701: mod_base = OTHER; note = thymine
  modified_base 3703: mod_base = OTHER; note = thymine
  modified_base 3705..3707: mod_base = OTHER; note = thymine
  modified_base 3717: mod_base = OTHER; note = thymine
  modified_base 3720: mod_base = OTHER; note = thymine
  modified_base 3723..3724: mod_base = OTHER; note = thymine
  modified_base 3726: mod_base = OTHER; note = thymine
  modified_base 3730..3731: mod_base = OTHER; note = thymine
  modified_base 3734: mod_base = OTHER; note = thymine
  modified_base 3740..3742: mod_base = OTHER; note = thymine
  modified_base 3747..3748: mod_base = OTHER; note = thymine
  modified_base 3752: mod_base = OTHER; note = thymine
  modified_base 3754: mod_base = OTHER; note = thymine
  modified_base 3756..3758: mod_base = OTHER; note = thymine
  modified_base 3763: mod_base = OTHER; note = thymine
  modified_base 3766: mod_base = OTHER; note = thymine
  modified_base 3769..3770: mod_base = OTHER; note = thymine
  modified_base 3772: mod_base = OTHER; note = thymine
  modified_base 3777: mod_base = OTHER; note = thymine
  modified_base 3779: mod_base = OTHER; note = thymine
  modified_base 3781: mod_base = OTHER; note = thymine
  modified_base 3789: mod_base = OTHER; note = thymine
  modified_base 3792: mod_base = OTHER; note = thymine
  modified_base 3794: mod_base = OTHER; note = thymine
  modified_base 3798: mod_base = OTHER; note = thymine
  modified_base 3806: mod_base = OTHER; note = thymine
  modified_base 3810..3811: mod_base = OTHER; note = thymine
  modified_base 3815: mod_base = OTHER; note = thymine
  modified_base 3821: mod_base = OTHER; note = thymine
  modified_base 3825..3827: mod_base = OTHER; note = thymine
  modified_base 3829: mod_base = OTHER; note = thymine
  modified_base 3834: mod_base = OTHER; note = thymine
  modified_base 3838..3841: mod_base = OTHER; note = thymine
  modified_base 3843: mod_base = OTHER; note = thymine
  modified_base 3845: mod_base = OTHER; note = thymine
  modified_base 3849..3850: mod_base = OTHER; note = thymine
  modified_base 3852: mod_base = OTHER; note = thymine
  modified_base 3859: mod_base = OTHER; note = thymine
  modified_base 3863..3864: mod_base = OTHER; note = thymine
  modified_base 3870..3871: mod_base = OTHER; note = thymine
  modified_base 3874..3876: mod_base = OTHER; note = thymine
  modified_base 3880: mod_base = OTHER; note = thymine
  modified_base 3883: mod_base = OTHER; note = thymine
  modified_base 3886: mod_base = OTHER; note = thymine
  modified_base 3897: mod_base = OTHER; note = thymine
  modified_base 3900: mod_base = OTHER; note = thymine
  modified_base 3903..3905: mod_base = OTHER; note = thymine
  modified_base 3908: mod_base = OTHER; note = thymine
  modified_base 3910: mod_base = OTHER; note = thymine
  modified_base 3917..3918: mod_base = OTHER; note = thymine
  modified_base 3922: mod_base = OTHER; note = thymine
  modified_base 3935: mod_base = OTHER; note = thymine
  modified_base 3937: mod_base = OTHER; note = thymine
  modified_base 3941: mod_base = OTHER; note = thymine
  modified_base 3943: mod_base = OTHER; note = thymine
  modified_base 3946..3947: mod_base = OTHER; note = thymine
  modified_base 3949: mod_base = OTHER; note = thymine
  modified_base 3951: mod_base = OTHER; note = thymine
  modified_base 3954: mod_base = OTHER; note = thymine
  modified_base 3957: mod_base = OTHER; note = thymine
  modified_base 3961: mod_base = OTHER; note = thymine
  modified_base 3968..3970: mod_base = OTHER; note = thymine
  modified_base 3972: mod_base = OTHER; note = thymine
  modified_base 3975: mod_base = OTHER; note = thymine
  modified_base 3978: mod_base = OTHER; note = thymine
  modified_base 3981: mod_base = OTHER; note = thymine
  modified_base 3983: mod_base = OTHER; note = thymine
  modified_base 3986..3987: mod_base = OTHER; note = thymine
  modified_base 3991..3992: mod_base = OTHER; note = thymine
  modified_base 3999: mod_base = OTHER; note = thymine
  modified_base 4002..4003: mod_base = OTHER; note = thymine
  modified_base 4010..4011: mod_base = OTHER; note = thymine
  modified_base 4019..4020: mod_base = OTHER; note = thymine
  modified_base 4023: mod_base = OTHER; note = thymine
  modified_base 4025..4026: mod_base = OTHER; note = thymine
  modified_base 4028..4029: mod_base = OTHER; note = thymine
  modified_base 4032: mod_base = OTHER; note = thymine
  modified_base 4037: mod_base = OTHER; note = thymine
  modified_base 4039..4040: mod_base = OTHER; note = thymine
  modified_base 4042..4043: mod_base = OTHER; note = thymine
  modified_base 4045: mod_base = OTHER; note = thymine
  modified_base 4050..4055: mod_base = OTHER; note = thymine
  modified_base 4058: mod_base = OTHER; note = thymine
  modified_base 4078: mod_base = OTHER; note = thymine
  modified_base 4080: mod_base = OTHER; note = thymine
  modified_base 4083: mod_base = OTHER; note = thymine
  modified_base 4088: mod_base = OTHER; note = thymine
  modified_base 4091..4092: mod_base = OTHER; note = thymine
  modified_base 4101: mod_base = OTHER; note = thymine
  modified_base 4105: mod_base = OTHER; note = thymine
  modified_base 4109..4111: mod_base = OTHER; note = thymine
  modified_base 4115: mod_base = OTHER; note = thymine
  modified_base 4121: mod_base = OTHER; note = thymine
  modified_base 4125: mod_base = OTHER; note = thymine
  modified_base 4134: mod_base = OTHER; note = thymine
  modified_base 4139: mod_base = OTHER; note = thymine
  modified_base 4143: mod_base = OTHER; note = thymine
  modified_base 4145..4147: mod_base = OTHER; note = thymine
  modified_base 4149: mod_base = OTHER; note = thymine
  modified_base 4155: mod_base = OTHER; note = thymine
  modified_base 4162: mod_base = OTHER; note = thymine
  modified_base 4170: mod_base = OTHER; note = thymine
  modified_base 4172: mod_base = OTHER; note = thymine
  modified_base 4176: mod_base = OTHER; note = thymine
  modified_base 4182: mod_base = OTHER; note = thymine
  modified_base 4185..4186: mod_base = OTHER; note = thymine
  modified_base 4191: mod_base = OTHER; note = thymine
  modified_base 4194: mod_base = OTHER; note = thymine
  modified_base 4198..4199: mod_base = OTHER; note = thymine
  modified_base 4206: mod_base = OTHER; note = thymine
  modified_base 4208: mod_base = OTHER; note = thymine
  modified_base 4211..4213: mod_base = OTHER; note = thymine
  modified_base 4216: mod_base = OTHER; note = thymine
  modified_base 4218: mod_base = OTHER; note = thymine
  modified_base 4220: mod_base = OTHER; note = thymine
  modified_base 4222: mod_base = OTHER; note = thymine
  modified_base 4229: mod_base = OTHER; note = thymine
  modified_base 4247: mod_base = OTHER; note = thymine
  modified_base 4251: mod_base = OTHER; note = thymine
  modified_base 4260: mod_base = OTHER; note = thymine
  modified_base 4262: mod_base = OTHER; note = thymine
  modified_base 4266: mod_base = OTHER; note = thymine
  modified_base 4269: mod_base = OTHER; note = thymine
  modified_base 4277: mod_base = OTHER; note = thymine
  modified_base 4281..4286: mod_base = OTHER; note = thymine
  modified_base 4289..4291: mod_base = OTHER; note = thymine
  modified_base 4296..4297: mod_base = OTHER; note = thymine
  modified_base 4300: mod_base = OTHER; note = thymine
  modified_base 4303: mod_base = OTHER; note = thymine
  modified_base 4305: mod_base = OTHER; note = thymine
  modified_base 4310..4313: mod_base = OTHER; note = thymine
  modified_base 4315: mod_base = OTHER; note = thymine
  modified_base 4320: mod_base = OTHER; note = thymine
  modified_base 4324: mod_base = OTHER; note = thymine
  modified_base 4326..4327: mod_base = OTHER; note = thymine
  modified_base 4333: mod_base = OTHER; note = thymine
  modified_base 4335..4336: mod_base = OTHER; note = thymine
  modified_base 4340: mod_base = OTHER; note = thymine
  modified_base 4343: mod_base = OTHER; note = thymine
  modified_base 4345: mod_base = OTHER; note = thymine
  modified_base 4348..4350: mod_base = OTHER; note = thymine
  modified_base 4352: mod_base = OTHER; note = thymine
  modified_base 4356: mod_base = OTHER; note = thymine
  modified_base 4359..4361: mod_base = OTHER; note = thymine
  modified_base 4363: mod_base = OTHER; note = thymine
  modified_base 4366: mod_base = OTHER; note = thymine
  modified_base 4371: mod_base = OTHER; note = thymine
  modified_base 4381..4382: mod_base = OTHER; note = thymine
  modified_base 4385..4386: mod_base = OTHER; note = thymine
  modified_base 4389: mod_base = OTHER; note = thymine
  modified_base 4392: mod_base = OTHER; note = thymine
  modified_base 4394: mod_base = OTHER; note = thymine
  modified_base 4396..4397: mod_base = OTHER; note = thymine
  modified_base 4402: mod_base = OTHER; note = thymine
  modified_base 4404: mod_base = OTHER; note = thymine
  modified_base 4407: mod_base = OTHER; note = thymine
  modified_base 4411: mod_base = OTHER; note = thymine
  modified_base 4415: mod_base = OTHER; note = thymine
  modified_base 4418: mod_base = OTHER; note = thymine
  modified_base 4421..4422: mod_base = OTHER; note = thymine
  modified_base 4424..4425: mod_base = OTHER; note = thymine
  modified_base 4427..4428: mod_base = OTHER; note = thymine
  modified_base 4431..4432: mod_base = OTHER; note = thymine
  modified_base 4435..4436: mod_base = OTHER; note = thymine
  modified_base 4438: mod_base = OTHER; note = thymine
  modified_base 4441..4442: mod_base = OTHER; note = thymine
  modified_base 4444..4445: mod_base = OTHER; note = thymine
  modified_base 4453: mod_base = OTHER; note = thymine
  modified_base 4456: mod_base = OTHER; note = thymine
  modified_base 4466: mod_base = OTHER; note = thymine
  modified_base 4468..4469: mod_base = OTHER; note = thymine
  modified_base 4473..4478: mod_base = OTHER; note = thymine
  modified_base 4481: mod_base = OTHER; note = thymine
  modified_base 4483..4484: mod_base = OTHER; note = thymine
  modified_base 4488: mod_base = OTHER; note = thymine
  modified_base 4491: mod_base = OTHER; note = thymine
  modified_base 4495: mod_base = OTHER; note = thymine
  modified_base 4500: mod_base = OTHER; note = thymine
  modified_base 4506: mod_base = OTHER; note = thymine
  modified_base 4512..4513: mod_base = OTHER; note = thymine
  modified_base 4515: mod_base = OTHER; note = thymine
  modified_base 4519: mod_base = OTHER; note = thymine
  modified_base 4523..4526: mod_base = OTHER; note = thymine
  modified_base 4528..4531: mod_base = OTHER; note = thymine
  modified_base 4537: mod_base = OTHER; note = thymine
  modified_base 4539: mod_base = OTHER; note = thymine
  modified_base 4546: mod_base = OTHER; note = thymine
  modified_base 4549: mod_base = OTHER; note = thymine
  modified_base 4559: mod_base = OTHER; note = thymine
  modified_base 4561..4562: mod_base = OTHER; note = thymine
  modified_base 4571..4573: mod_base = OTHER; note = thymine
  modified_base 4575: mod_base = OTHER; note = thymine
  modified_base 4578..4579: mod_base = OTHER; note = thymine
  modified_base 4581: mod_base = OTHER; note = thymine
  modified_base 4584: mod_base = OTHER; note = thymine
  modified_base 4597: mod_base = OTHER; note = thymine
  modified_base 4599..4601: mod_base = OTHER; note = thymine
  modified_base 4607..4608: mod_base = OTHER; note = thymine
  modified_base 4611: mod_base = OTHER; note = thymine
  modified_base 4617..4618: mod_base = OTHER; note = thymine
  modified_base 4624..4625: mod_base = OTHER; note = thymine
  modified_base 4633..4636: mod_base = OTHER; note = thymine
  modified_base 4638..4639: mod_base = OTHER; note = thymine
  modified_base 4642..4643: mod_base = OTHER; note = thymine
  modified_base 4650: mod_base = OTHER; note = thymine
  modified_base 4658: mod_base = OTHER; note = thymine
  modified_base 4660: mod_base = OTHER; note = thymine
  modified_base 4663: mod_base = OTHER; note = thymine
  modified_base 4666: mod_base = OTHER; note = thymine
  modified_base 4669: mod_base = OTHER; note = thymine
  modified_base 4671..4672: mod_base = OTHER; note = thymine
  modified_base 4678: mod_base = OTHER; note = thymine
  modified_base 4680: mod_base = OTHER; note = thymine
  modified_base 4686..4687: mod_base = OTHER; note = thymine
  modified_base 4690..4693: mod_base = OTHER; note = thymine
  modified_base 4697..4698: mod_base = OTHER; note = thymine
  modified_base 4703: mod_base = OTHER; note = thymine
  modified_base 4705: mod_base = OTHER; note = thymine
  modified_base 4709..4711: mod_base = OTHER; note = thymine
  modified_base 4715: mod_base = OTHER; note = thymine
  modified_base 4717..4718: mod_base = OTHER; note = thymine
  modified_base 4721: mod_base = OTHER; note = thymine
  modified_base 4723: mod_base = OTHER; note = thymine
  modified_base 4725..4726: mod_base = OTHER; note = thymine
  modified_base 4738: mod_base = OTHER; note = thymine
  modified_base 4753: mod_base = OTHER; note = thymine
  modified_base 4755: mod_base = OTHER; note = thymine
  modified_base 4757..4759: mod_base = OTHER; note = thymine
  modified_base 4766: mod_base = OTHER; note = thymine
  modified_base 4769: mod_base = OTHER; note = thymine
  modified_base 4774..4776: mod_base = OTHER; note = thymine
  modified_base 4780: mod_base = OTHER; note = thymine
  modified_base 4782: mod_base = OTHER; note = thymine
  modified_base 4784: mod_base = OTHER; note = thymine
  modified_base 4792: mod_base = OTHER; note = thymine
  modified_base 4794..4795: mod_base = OTHER; note = thymine
  modified_base 4800: mod_base = OTHER; note = thymine
  modified_base 4802..4804: mod_base = OTHER; note = thymine
  source 1..7114: mol_type = other RNA
cgcacagtggatcctaggcgtaacttcatcattaaaatctcagattctgctctgagtgtgacttactgcgaagaggcagacaaatgggcaactgcaacggggcatccaagtctaaccagccagactcctcaagagccacacagccagccgcagaatttaggagggtagctcacagcagtctatatggtagatataactgtaagtgctgctggtttgctgataccaatttgataacctgtaatgatcactacctttgtttaaggtgccatcagggtatgttaaggaattcagatctctgcaatatctgctggaagcccctgcccaccacaatcacagtaccggtggagccaacagcaccaccaccataggcagactgcacagggtcagacccgaccccccggggggcccccatggggaccccccgtgggggaaccccgggggtgatgcgccattagtcaatgtctttgatctcgactttgtgcttcagtggcctgcatgtcacccctttcaatctgaactgcccttggggatctgatatcagcaggtcatttaaagatctgctgaatgccaccttgaaatttgagaattccaaccagtcaccaaatttatcaagtgaacggatcaactgctctttgtgtagatcataaacgaggacaaagtcctcttgctgaaataatattgtttgtgatgttgtttttagataaggccatagttggcttaataaggtttccacactatcaatgtcctctagtgctccaattgccttgactatgacatccccagacaactcaactctatatgttgacaacctttcattacctctgtaaaagataccctctttcaagacaagaggttctcctgggttatctggcccaatgaggtcatatgcatacttgttacttagttcagaataaaagtcaccaaagttgaacttaacatggctcagaatattgtcatcatttgtcgcagcgtagcctgcatcaataaacaagccagctaggtcaaagctctcatggcctgtgaacaatggtaggctagcgataaccagtgcaccatccaacaatgagtggcttccctcagacccagaaacacattgactcattgcatccacattcagctctaattcaggggtaccgacatcatccactcctagtgaactgacaatggtgtaactgtacaccatctttcttctaagtttaaattttgtcgaaactcgtgtgtgttctacttgaatgatcaattttagtttcacagcttcttggcaagcaacattgcgcaacacagtgtgcaggtccatcatgtcttcctgaggcaacaaggagatgttgtcaacagagacaccctcaaggaaaaccttgatattatcaaagctagaaactacataacccattgcaatgtcttcaacaaacattgctcttgatactttattattcctaactgacaaggtaaaatctgtgagttcagctagatctacttgactgtcatcttctagatctagaacttcattgaaccaaaagaaggatttgagacacgatgttgacatgactagtgggtttatcatcgaagataagacaacttgcaccatgaagttcctgcaaacttgctgtgggctgatgccaacttcccaatttgtatactctgactgtctaacatgggctgaagcgcaatcactctgtttcacaatataaacattattatctcttactttcaataagtgacttataatccctaagttttcattcatcatgtctagagccacacagacatctagaaacttgagtcttccactatccaaagatctgttcacttgaagatcattcataaagggtgccaaatgttcttcaaatagtttggggtaatttcttcgtatagaatgcaatacatggttcatgcctaattggtcttctatctgtcgtactgctttgggtttaacagcccagaagaaattcttattacataagaccagaggggcctgtggactcttaatagcaaaaaacacccactcccctaactcacaggcatttgtcagcaccaaagagaagtaatcccacaaaattggtttagaaaattggttaacttctttaagtgatttttgacagtaaataactttaggctttctctcacaaattccacaaagacatggcattattcgagtaaatatgtcctttatatacagaaatccgcctttaccatccctaacacacttactccccatactcttacaaaacccaatgaagcctgaggcaacagaagactgaaatgcagatttgttgattgactctgccaagatcttcttcacgccttttgtgaaatttcttgacagcctggactgtattgtccttatcaatgttggcatctcttctttctctaacactcttcgacttgtcatgagtttggtcctcaagaccaacctcaagtccccaaagctcgctaaattgacccatctgtagtctagagtttgtctgatttcatcttcactacacccggcatattgcaggaatccggataaagcctcatcccctcccctgcttatcaagttgataaggttttcctcaaagattttgcctctcttaatgtcattgaacactttcctcgcgcagttccttataaacattgtctccttatcatcagaaaaaatagcttcaattttcctctgtagacggtaccctctagacccatcaacccagtctttgacatcttgttcttcaatagctccaaacggagtctctctgtatccagagtatctaatcaattggttgactctaatggaaatctttgacactatatgagtgctaaccccattagcaatacattgatcacaaattgtgtctatggtctctgacagttgtgttggagttttacacttaacgttgtgtagagcagcagacacaaacttggtgagtaaaggagtctcttcacccatgacaaaaaatcttgacttaaactcagcaacaaaagttcctatcacactctttgggctgataaacttgtttaatttagaagataagaattcatggaagcacaccatttccagcagttctgtcctgtcttgaaacttttcatcactaaggcaaggaatttttataaggctaacctggtcatcgctggaggtataagtgacaggtatcacatcatacaataagtcaagtgcataacacagaaattgttcagtaattagcccatataaatctgatgtgttgtgcaagattccctggcccatgtccaagacagacattatatggctggggacctggtcccttgactgcagatactggtgaaaaaactcttcaccaacactagtacagtcacaacccattaaacctaaagatctcttcaatttccctacacagtaggcttctgcaacattaattggaacttcaacgaccttatgaagatgccatttgagaatgttcattactggttcaagattcacctttgttctatctctgggattcttcaattctaatgtgtacaaaaaagaaaggaaaagtgctgggctcatagttggtccccatttggagtggtcatatgaacaggacaagtcaccattgttaacagccattttcatatcacagattgcacgttcgaattccttttctgaattcaagcatgtgtatttcattgaactacccacagcttctgagaagtcttcaactaacctggtcatcagcttagtgttgaggtctcccacatacagttctctatttgagccaacctgctccttataacttagtccaaatttcaagttccctgtatttgagctgatgcttgtgaactctgtaggagagtcgtctgaatagaaacataaattccgtagggctgcatttgtaaaataacttttgtctagcttatcagcaatggcttcagaattgctttccctggtactaagccgaacctcatcctttagtctcagaacttcactggaaaagcccaatctagatctacttctatgctcataactacccaatttctgatcataatgtccttgaattaaaagatacttgaagcattcaaagaattcatcttcttggtaggctattgttgtcaaattttttaataacaaacccaaagggcagatgtcctgcggtgcttcaagaaaataagtcaatttaaatggagatagataaacagcatcacataactctttatacacatcagacctgagcacatctggatcaaaatccttcacctcatgcattgacacctctgctttaatctctctcaacactccaaaaggggcccacaatgactcaagagactctcgctcatcaacagatggattttttgatttcaacttggtgatctcaacttttgtcccctcactattagccatcttggctagtgtcatttgtacgtcatttctaataccctcaaaggcccttacttgatcctctgttaaactctcatacatcactgataattcttcttgattggttctggttcttgaaccggtgctcacaagacctgttagattttttaatattaagtagtccatggaatcaggatcaagattatacctgccttttgttttaaacctctcagccatagtagaaacgcatgttgaaacaagtttctccttatcataaacagaaagaatatttccaagttcgtcgagcttggggattaccacacttttattgcttgacagatccagagctgtgctagtgatgttaggcctgtagggattgcttttcagttcacctgtaactttaagtcttcctctattgaagagagaaatgcagaaggacaaaatctctttacacactcctggaatttgagtatctgaggaagtcttagcctctttggaaaagaatctgtccaatcctcttatcatggtgtcctcttgttccagtgttagactcccacttagaggggggtttacaacaacacaatcaaacttgactttgggctcaataaacttctcaaaacacttgatttgatctgtcaggcgatcaggtgtctctttggttaccaagtgacacagataactaacatttaatagatatttaaaccttcttgcaaagtaaagatctgcatcttccccttcacccaaaattgtctggaaaagttccacagccatcctctgaatcagcacctctgatccagacatgcagtcgacccttaactttgacatcaaatccacatgatggatttgatttgcatatgccatcaagaaatatcttagaccttgtaaaaatgtctggttccttttggaaggggaacagagtacagctaacactaacaatcttaatattggccttgtcattgtcatgagttcgtggctaaaatccaaccagctggtcatttcctcacacatttcaattaacacatcctccgaaaatataggcaggaaaaatctctttggatcacagtaaaaagagccttgttcttccaataccccattgatggatagatagatagaatagcaccttgacttctcacctgttttttggtaaaacaagagaccaaatgtattctttgtcagatgaaatctttgtacataacactctcttagtctaacattcccaaaatatctagaatactctctttcattgattaacaatcgggaggaaaatgatgtcttcatcgagttgaccaatgcaagggaaatggaggacaaaatcctaaataatttcttctgctcaccttccactaagctgctgaatggctgatgtctacagattttctcaaattccttgttaatagtatatctcatcactggtctgtcagaaacaagtgcctgagctaaaatcatcaagctatccatatcagggtgttttattagtttttccagctgtgaccagagatcttgatgagagttcttcaatgttctggaacacgcttgaacccacttggggctggtcatcaatttcttccttattagtttaatcgcctccagaatatctagaagtctgtcattgactaacattaacatttgtccaacaactattcccgcatttcttaaccttacaattgcatcatcatgcgttttgaaaagatcacaaagtaaattgagtaaaactaagtccagaaacagtaaagtgtttctcctggtgttgaaaacttttagacctttcactttgttacacacggaaagggcttgaagataacacctctctacagcatcaatagatatagaattctcatctgactggctttccatgttgacttcatctattggatgcaatgcgatagagtagactacatccatcaacttgtttgcacaaaaagggcagctgggcacatcactgtctttgtggcttcctaataagatcaagtcatttataagcttagacttttgtgaaaatttgaatttccccaactgcttgtcaaaaatctccttcttaaaccaaaaccttaactttatgagttcttctcttatgacagattctctaatgtctcctctaaccccaacaaagagggattcatttaacctctcatcataacccaaagaattctttttcaagcattcgatgttttctaatcccaagctctggttttttgtgttggacaaactatggatcaatcgctggtattcttgttcttcaatattaatctcttgcataaattttgatttctttaggatgtcgatcagcaaccaccgaactctttcaacaacccaatcagcaaggaatctattgctgtagctagatctgccatcaaccacaggaaccaacgtaatccctgcccttagtaggtcggactttaggtttaagagctttgacatgtcactcttccattttctctcaaactcatcaggattgaccctaacaaaggtttccaataggatgagtgttttccctgtgagtttgaagccatccggaatgacttttggaagggtgggacatagtatgccatagtcagacaggatcacatcaacaaacttctgatctgaattgatctgacaggcgtgtgcctcacaggactcaagctctactaaacttgacagaagtttgaacccttccaacaacagagagctggggtgatgttgagataaaaagatgtccctttggtatgctagctcctgtctttctggaaaatgctttctaataaggctttttatttcatttactgattcctccatgctcaagtgccgcctaggatccactgtgcg

SEQ ID NO: 3 - RNA, 3410 nt, unidentified
  modified_base 8: mod_base = OTHER; note = thymine
  modified_base 12: mod_base = OTHER; note = thymine
  modified_base 15: mod_base = OTHER; note = thymine
  modified_base 22..25: mod_base = OTHER; note = thymine
  modified_base 28..29: mod_base = OTHER; note = thymine
  modified_base 34: mod_base = OTHER; note = thymine
  modified_base 36: mod_base = OTHER; note = thymine
  modified_base 39..40: mod_base = OTHER; note = thymine
  modified_base 42: mod_base = OTHER; note = thymine
  modified_base 46: mod_base = OTHER; note = thymine
  modified_base 48..51: mod_base = OTHER; note = thymine
  modified_base 53: mod_base = OTHER; note = thymine
  modified_base 55..57: mod_base = OTHER; note = thymine
  modified_base 66: mod_base = OTHER; note = thymine
  modified_base 75: mod_base = OTHER; note = thymine
  modified_base 79..80: mod_base = OTHER; note = thymine
  modified_base 87: mod_base = OTHER; note = thymine
  modified_base 95..96: mod_base = OTHER; note = thymine
  modified_base 99..100: mod_base = OTHER; note = thymine
  modified_base 104..105: mod_base = OTHER; note = thymine
  modified_base 108: mod_base = OTHER; note = thymine
  modified_base 117: mod_base = OTHER; note = thymine
  modified_base 125..129: mod_base = OTHER; note = thymine
  modified_base 139: mod_base = OTHER; note = thymine
  modified_base 141: mod_base = OTHER; note = thymine
  modified_base 147..148: mod_base = OTHER; note = thymine
  modified_base 151: mod_base = OTHER; note = thymine
  modified_base 153..154: mod_base = OTHER; note = thymine
  modified_base 156..157: mod_base = OTHER; note = thymine
  modified_base 162: mod_base = OTHER; note = thymine
  modified_base 166: mod_base = OTHER; note = thymine
  modified_base 168: mod_base = OTHER; note = thymine
  modified_base 171..172: mod_base = OTHER; note = thymine
  modified_base 177: mod_base = OTHER; note = thymine
  modified_base 180..181: mod_base = OTHER; note = thymine
  modified_base 187: mod_base = OTHER; note = thymine
  modified_base 189: mod_base = OTHER; note = thymine
  modified_base 192: mod_base = OTHER; note = thymine
  modified_base 198: mod_base = OTHER; note = thymine
  modified_base 200: mod_base = OTHER; note = thymine
  modified_base 208: mod_base = OTHER; note = thymine
  modified_base 211..213: mod_base = OTHER; note = thymine
  modified_base 215..216: mod_base = OTHER; note = thymine
  modified_base 221..222: mod_base = OTHER; note = thymine
  modified_base 224..226: mod_base = OTHER; note = thymine
  modified_base 228: mod_base = OTHER; note = thymine
  modified_base 230..231: mod_base = OTHER; note = thymine
  modified_base 234: mod_base = OTHER; note = thymine
  modified_base 240: mod_base = OTHER; note = thymine
  modified_base 251: mod_base = OTHER; note = thymine
  modified_base 254: mod_base = OTHER; note = thymine
  modified_base 268..271: mod_base = OTHER; note = thymine
  modified_base 276: mod_base = OTHER; note = thymine
  modified_base 282: mod_base = OTHER; note = thymine
  modified_base 293..294: mod_base = OTHER; note = thymine
  modified_base 301: mod_base = OTHER; note = thymine
  modified_base 303: mod_base = OTHER; note = thymine
  modified_base 305: mod_base = OTHER; note = thymine
  modified_base 308..309: mod_base = OTHER; note = thymine
  modified_base 311: mod_base = OTHER; note = thymine
  modified_base 315: mod_base = OTHER; note = thymine
  modified_base 318: mod_base = OTHER; note = thymine
  modified_base 322: mod_base = OTHER; note = thymine
  modified_base 324: mod_base = OTHER; note = thymine
  modified_base 326..329: mod_base = OTHER; note = thymine
  modified_base 337: mod_base = OTHER; note = thymine
  modified_base 343: mod_base = OTHER; note = thymine
  modified_base 348: mod_base = OTHER; note = thymine
  modified_base 352: mod_base = OTHER; note = thymine
  modified_base 354: mod_base = OTHER; note = thymine
  modified_base 356..357: mod_base = OTHER; note = thymine
  modified_base 359: mod_base = OTHER; note = thymine
  modified_base 361: mod_base = OTHER; note = thymine
  modified_base 365..366: mod_base = OTHER; note = thymine
  modified_base 379: mod_base = OTHER; note = thymine
  modified_base 381..383: mod_base = OTHER; note = thymine
  modified_base 387..388: mod_base = OTHER; note = thymine
  modified_base 400: mod_base = OTHER; note = thymine
  modified_base 403..404: mod_base = OTHER; note = thymine
  modified_base 407..409: mod_base = OTHER; note = thymine
  modified_base 414: mod_base = OTHER; note = thymine
  modified_base 419..421: mod_base = OTHER; note = thymine
  modified_base 424: mod_base = OTHER; note = thymine
  modified_base 427: mod_base = OTHER; note = thymine
  modified_base 429..430: mod_base = OTHER; note = thymine
  modified_base 435: mod_base = OTHER; note = thymine
  modified_base 437..438: mod_base = OTHER; note = thymine
  modified_base 440..441: mod_base = OTHER; note = thymine
  modified_base 449: mod_base = OTHER; note = thymine
  modified_base 451: mod_base = OTHER; note = thymine
  modified_base 454: mod_base = OTHER; note = thymine
  modified_base 456..457: mod_base = OTHER; note = thymine
  modified_base 459: mod_base = OTHER; note = thymine
  modified_base 462: mod_base = OTHER; note = thymine
  modified_base 469: mod_base = OTHER; note = thymine
  modified_base 478: mod_base = OTHER; note = thymine
  modified_base 480: mod_base = OTHER; note = thymine
  modified_base 485: mod_base = OTHER; note = thymine
  modified_base 488: mod_base = OTHER; note = thymine
  modified_base 490..491: mod_base = OTHER; note = thymine
  modified_base 499: mod_base = OTHER; note = thymine
  modified_base 502: mod_base = OTHER; note = thymine
  modified_base 505: mod_base = OTHER; note = thymine
  modified_base 510..512: mod_base = OTHER; note = thymine
  modified_base 515..516: mod_base = OTHER; note = thymine
  modified_base 518: mod_base = OTHER; note = thymine
  modified_base 520: mod_base = OTHER; note = thymine
  modified_base 524: mod_base = OTHER; note = thymine
  modified_base 527..528: mod_base = OTHER; note = thymine
  modified_base 531: mod_base = OTHER; note = thymine
  modified_base 535: mod_base = OTHER; note = thymine
  modified_base 538: mod_base = OTHER; note = thymine
  modified_base 540: mod_base = OTHER; note = thymine
  modified_base 547: mod_base = OTHER; note = thymine
  modified_base 550..551: mod_base = OTHER; note = thymine
  modified_base 556: mod_base = OTHER; note = thymine
  modified_base 558: mod_base = OTHER; note = thymine
  modified_base 569..571: mod_base = OTHER; note = thymine
  modified_base 573: mod_base = OTHER; note = thymine
  modified_base 575: mod_base = OTHER; note = thymine
  modified_base 577: mod_base = OTHER; note = thymine
  modified_base 586: mod_base = OTHER; note = thymine
  modified_base 602..603: mod_base = OTHER; note = thymine
  modified_base 606: mod_base = OTHER; note = thymine
  modified_base 608..610: mod_base = OTHER; note = thymine
  modified_base 615: mod_base = OTHER; note = thymine
  modified_base 623: mod_base = OTHER; note = thymine
  modified_base 631: mod_base = OTHER; note = thymine
  modified_base 634: mod_base = OTHER; note = thymine
  modified_base 636: mod_base = OTHER; note = thymine
  modified_base 640: mod_base = OTHER; note = thymine
  modified_base 646..647: mod_base = OTHER; note = thymine
  modified_base 649: mod_base = OTHER; note = thymine
  modified_base 652: mod_base = OTHER; note = thymine
  modified_base 659..661: mod_base = OTHER; note = thymine
  modified_base 667: mod_base = OTHER; note = thymine
  modified_base 672: mod_base = OTHER; note = thymine
  modified_base 676: mod_base = OTHER; note = thymine
  modified_base 679..681: mod_base = OTHER; note = thymine
  modified_base 683: mod_base = OTHER; note = thymine
  modified_base 685: mod_base = OTHER; note = thymine
  modified_base 692: mod_base = OTHER; note = thymine
  modified_base 694: mod_base = OTHER; note = thymine
  modified_base 697: mod_base = OTHER; note = thymine
  modified_base 703..704: mod_base = OTHER; note = thymine
  modified_base 707..708: mod_base = OTHER; note = thymine
  modified_base 712: mod_base = OTHER; note = thymine
  modified_base 720: mod_base = OTHER; note = thymine
  modified_base 722: mod_base = OTHER; note = thymine
  modified_base 725..726: mod_base = OTHER; note = thymine
  modified_base 729: mod_base = OTHER; note = thymine
  modified_base 742: mod_base = OTHER; note = thymine
  modified_base 748..749: mod_base = OTHER; note = thymine
  modified_base 754: mod_base = OTHER; note = thymine
  modified_base 756: mod_base = OTHER; note = thymine
  modified_base 761: mod_base = OTHER; note = thymine
  modified_base 763: mod_base = OTHER; note = thymine
  modified_base 768: mod_base = OTHER; note = thymine
  modified_base 777..778: mod_base = OTHER; note = thymine
  modified_base 785..786: mod_base = OTHER; note = thymine
  modified_base 791..792: mod_base = OTHER; note = thymine
  modified_base 795..796: mod_base = OTHER; note = thymine
  modified_base 805: mod_base = OTHER; note = thymine
  modified_base 813..814: mod_base = OTHER; note = thymine
  modified_base 819..820: mod_base = OTHER; note = thymine
  modified_base 830: mod_base = OTHER; note = thymine
  modified_base 833..834: mod_base = OTHER; note = thymine
  modified_base 842..843: mod_base = OTHER; note = thymine
  modified_base 845..846: mod_base = OTHER; note = thymine
  modified_base 848: mod_base = OTHER; note = thymine
  modified_base 851: mod_base = OTHER; note = thymine
  modified_base 854: mod_base = OTHER; note = thymine
  modified_base 856..858: mod_base = OTHER; note = thymine
  modified_base 866: mod_base = OTHER; note = thymine
  modified_base 869: mod_base = OTHER; note = thymine
  modified_base 880: mod_base = OTHER; note = thymine
  modified_base 886: mod_base = OTHER; note = thymine
  modified_base 893: mod_base = OTHER; note = thymine
  modified_base 895..896: mod_base = OTHER; note = thymine
  modified_base 898: mod_base = OTHER; note = thymine
  modified_base 900: mod_base = OTHER; note = thymine
  modified_base 908: mod_base = OTHER; note = thymine
  modified_base 912: mod_base = OTHER; note = thymine
  modified_base 915: mod_base = OTHER; note = thymine
  modified_base 918..919: mod_base = OTHER; note = thymine
  modified_base 930: mod_base = OTHER; note = thymine
  modified_base 935: mod_base = OTHER; note = thymine
  modified_base 937..940: mod_base = OTHER; note = thymine
  modified_base 946: mod_base = OTHER; note = thymine
  modified_base 949: mod_base = OTHER; note = thymine
  modified_base 952: mod_base = OTHER; note = thymine
  modified_base 954: mod_base = OTHER; note = thymine
  modified_base 962: mod_base = OTHER; note = thymine
  modified_base 967: mod_base = OTHER; note = thymine
  modified_base 969: mod_base = OTHER; note = thymine
  modified_base 973: mod_base = OTHER; note = thymine
  modified_base 976: mod_base = OTHER; note = thymine
  modified_base 980: mod_base = OTHER; note = thymine
  modified_base 982: mod_base = OTHER; note = thymine
  modified_base 986..987: mod_base = OTHER; note = thymine
  modified_base 989: mod_base = OTHER; note = thymine
  modified_base 991: mod_base = OTHER; note = thymine
  modified_base 996: mod_base = OTHER; note = thymine
  modified_base 999: mod_base = OTHER; note = thymine
  modified_base 1005..1009: mod_base = OTHER; note = thymine
  modified_base 1012..1013: mod_base = OTHER; note = thymine
  modified_base 1024: mod_base = OTHER; note = thymine
  modified_base 1027: mod_base = OTHER; note = thymine
  modified_base 1029: mod_base = OTHER; note = thymine
  modified_base 1037..1038: mod_base = OTHER; note = thymine
  modified_base 1042: mod_base = OTHER; note = thymine
  modified_base 1045: mod_base = OTHER; note = thymine
  modified_base 1051: mod_base = OTHER; note = thymine
  modified_base 1062: mod_base = OTHER; note = thymine
  modified_base 1066: mod_base = OTHER; note = thymine
  modified_base 1068: mod_base = OTHER; note = thymine
  modified_base 1071: mod_base = OTHER; note = thymine
  modified_base 1083: mod_base = OTHER; note = thymine
  modified_base 1087: mod_base = OTHER; note = thymine
  modified_base 1092: mod_base = OTHER; note = thymine
  modified_base 1095: mod_base = OTHER; note = thymine
  modified_base 1097: mod_base = OTHER; note = thymine
  modified_base 1099: mod_base = OTHER; note = thymine
  modified_base 1105..1107: mod_base = OTHER; note = thymine
  modified_base 1109..1110: mod_base = OTHER; note = thymine
  modified_base 1113: mod_base = OTHER; note = thymine
  modified_base 1125..1126: mod_base = OTHER; note = thymine
  modified_base 1134: mod_base = OTHER; note = thymine
  modified_base 1137: mod_base = OTHER; note = thymine
  modified_base 1141: mod_base = OTHER; note = thymine
  modified_base 1143: mod_base = OTHER; note = thymine
  modified_base 1147..1148: mod_base = OTHER; note = thymine
  modified_base 1151: mod_base = OTHER; note = thymine
  modified_base 1156..1157: mod_base = OTHER; note = thymine
  modified_base 1166..1169: mod_base = OTHER; note = thymine
  modified_base 1172: mod_base = OTHER; note = thymine
  modified_base 1174: mod_base = OTHER; note = thymine
  modified_base 1176: mod_base = OTHER; note = thymine
  modified_base 1188..1190: mod_base = OTHER; note = thymine
  modified_base 1202: mod_base = OTHER; note = thymine
  modified_base 1205..1206: mod_base = OTHER; note = thymine
  modified_base 1214: mod_base = OTHER; note = thymine
  modified_base 1217: mod_base = OTHER; note = thymine
  modified_base 1220..1221: mod_base = OTHER; note = thymine
  modified_base 1224: mod_base = OTHER; note = thymine
  modified_base 1238: mod_base = OTHER; note = thymine
  modified_base 1240..1242: mod_base = OTHER; note = thymine
  modified_base 1248: mod_base = OTHER; note = thymine
  modified_base 1250: mod_base = OTHER; note = thymine
  modified_base 1252: mod_base = OTHER; note = thymine
  modified_base 1256..1257: mod_base = OTHER; note = thymine
  modified_base 1261: mod_base = OTHER; note = thymine
  modified_base 1264: mod_base = OTHER; note = thymine
  modified_base 1268: mod_base = OTHER; note = thymine
  modified_base 1272: mod_base = OTHER; note = thymine
  modified_base 1274..1275: mod_base = OTHER; note = thymine
  modified_base 1282: mod_base = OTHER; note = thymine
  modified_base 1286..1287: mod_base = OTHER; note = thymine
  modified_base 1289..1290: mod_base = OTHER; note = thymine
  modified_base 1293..1295: mod_base = OTHER; note = thymine
  modified_base 1297: mod_base = OTHER; note = thymine
  modified_base 1302: mod_base = OTHER; note = thymine
  modified_base 1305: mod_base = OTHER; note = thymine
  modified_base 1316: mod_base = OTHER; note = thymine
  modified_base 1318..1319: mod_base = OTHER; note = thymine
  modified_base 1333: mod_base = OTHER; note = thymine
  modified_base 1339: mod_base = OTHER; note = thymine
  modified_base 1342..1344: mod_base = OTHER; note = thymine
  modified_base 1348..1350: mod_base = OTHER; note = thymine
  modified_base 1353..1354: mod_base = OTHER; note = thymine
  modified_base 1359: mod_base = OTHER; note = thymine
  modified_base 1361: mod_base = OTHER; note = thymine
  modified_base 1363: mod_base = OTHER; note = thymine
  modified_base 1365..1367: mod_base = OTHER; note = thymine
  modified_base 1377: mod_base = OTHER; note = thymine
  modified_base 1379..1380: mod_base = OTHER; note = thymine
  modified_base 1382..1383: mod_base = OTHER; note = thymine
  modified_base 1391: mod_base = OTHER; note = thymine
  modified_base 1395: mod_base = OTHER; note = thymine
  modified_base 1397..1398: mod_base = OTHER; note = thymine
  modified_base 1401..1402: mod_base = OTHER; note = thymine
  modified_base 1406..1407: mod_base = OTHER; note = thymine
  modified_base 1410: mod_base = OTHER; note = thymine
  modified_base 1414: mod_base = OTHER; note = thymine
  modified_base 1416: mod_base = OTHER; note = thymine
  modified_base 1421: mod_base = OTHER; note = thymine
  modified_base 1434: mod_base = OTHER; note = thymine
  modified_base 1448: mod_base = OTHER; note = thymine
  modified_base 1453..1455: mod_base = OTHER; note = thymine
  modified_base 1466..1467: mod_base = OTHER; note = thymine
  modified_base 1475..1476: mod_base = OTHER; note = thymine
  modified_base 1480: mod_base = OTHER; note = thymine
  modified_base 1483..1484: mod_base = OTHER; note = thymine
  modified_base 1490: mod_base = OTHER; note = thymine
  modified_base 1492: mod_base = OTHER; note = thymine
  modified_base 1495: mod_base = OTHER; note = thymine
  modified_base 1499: mod_base = OTHER; note = thymine
  modified_base 1507: mod_base = OTHER; note = thymine
  modified_base 1509: mod_base = OTHER; note = thymine
  modified_base 1524..1526: mod_base = OTHER; note = thymine
  modified_base 1531: mod_base = OTHER; note = thymine
  modified_base 1541: mod_base = OTHER; note = thymine
  modified_base 1548: mod_base = OTHER; note = thymine
  modified_base 1551: mod_base = OTHER; note = thymine
  modified_base 1557: mod_base = OTHER; note = thymine
  modified_base 1576: mod_base = OTHER; note = thymine
  modified_base 1585: mod_base = OTHER; note = thymine
  modified_base 1588: mod_base = OTHER; note = thymine
  modified_base 1590: mod_base = OTHER; note = thymine
  modified_base 1601: mod_base = OTHER; note = thymine
  modified_base 1610: mod_base = OTHER; note = thymine
  modified_base 1621: mod_base = OTHER; note = thymine
  modified_base 1632: mod_base = OTHER; note = thymine
  modified_base 1635..1636: mod_base = OTHER; note = thymine
  modified_base 1641: mod_base = OTHER; note = thymine
  modified_base 1645: mod_base = OTHER; note = thymine
  modified_base 1650: mod_base = OTHER; note = thymine
  modified_base 1654..1655: mod_base = OTHER; note = thymine
  modified_base 1673: mod_base = OTHER; note = thymine
  modified_base 1677: mod_base = OTHER; note = thymine
  modified_base 1680: mod_base = OTHER; note = thymine
  modified_base 1683: mod_base = OTHER; note = thymine
  modified_base 1689: mod_base = OTHER; note = thymine
  modified_base 1691: mod_base = OTHER; note = thymine
  modified_base 1695..1696: mod_base = OTHER; note = thymine
  modified_base 1699: mod_base = OTHER; note = thymine
  modified_base 1702: mod_base = OTHER; note = thymine
  modified_base 1705: mod_base = OTHER; note = thymine
  modified_base 1711..1712: mod_base = OTHER; note = thymine
  modified_base 1717: mod_base = OTHER; note = thymine
  modified_base 1719: mod_base = OTHER; note = thymine
  modified_base 1725: mod_base = OTHER; note = thymine
  modified_base 1729: mod_base = OTHER; note = thymine
  modified_base 1736..1737: mod_base = OTHER; note = thymine
  modified_base 1739: mod_base = OTHER; note = thymine
  modified_base 1744: mod_base = OTHER; note = thymine
  modified_base 1746: mod_base = OTHER; note = thymine
  modified_base 1759: mod_base = OTHER; note = thymine
  modified_base 1761: mod_base = OTHER; note = thymine
  modified_base 1764..1765: mod_base = OTHER; note = thymine
  modified_base 1769: mod_base = OTHER; note = thymine
  modified_base 1780..1781: mod_base = OTHER; note = thymine
  modified_base 1790: mod_base = OTHER; note = thymine
  modified_base 1792: mod_base = OTHER; note = thymine
  modified_base 1795..1798: mod_base = OTHER; note = thymine
  modified_base 1802: mod_base = OTHER; note = thymine
  modified_base 1804..1808: mod_base = OTHER; note = thymine
  modified_base 1810..1811: mod_base = OTHER; note = thymine
  modified_base 1813..1814: mod_base = OTHER; note = thymine
  modified_base 1824: mod_base = OTHER; note = thymine
  modified_base 1828..1829: mod_base = OTHER; note = thymine
  modified_base 1831: mod_base = OTHER; note = thymine
  modified_base 1834: mod_base = OTHER; note = thymine
  modified_base 1836..1838: mod_base = OTHER; note = thymine
  modified_base 1842: mod_base = OTHER; note = thymine
  modified_base 1846: mod_base = OTHER; note = thymine
  modified_base 1852: mod_base = OTHER; note = thymine
  modified_base 1854..1856: mod_base = OTHER; note = thymine
  modified_base 1858: mod_base = OTHER; note = thymine
  modified_base 1867: mod_base = OTHER; note = thymine
  modified_base 1869..1870: mod_base = OTHER; note = thymine
  modified_base 1873: mod_base = OTHER; note = thymine
  modified_base 1879: mod_base = OTHER; note = thymine
  modified_base 1883: mod_base = OTHER; note = thymine
  modified_base 1887..1888: mod_base = OTHER; note = thymine
  modified_base 1890..1891: mod_base = OTHER; note = thymine
  modified_base 1896: mod_base = OTHER; note = thymine
  modified_base 1898: mod_base = OTHER; note = thymine
  modified_base 1903..1904: mod_base = OTHER; note = thymine
  modified_base 1909: mod_base = OTHER; note = thymine
  modified_base 1917..1919: mod_base = OTHER; note = thymine
  modified_base 1924: mod_base = OTHER; note = thymine
  modified_base 1926: mod_base = OTHER; note = thymine
  modified_base 1928..1929: mod_base = OTHER; note = thymine
  modified_base 1931..1932: mod_base = OTHER; note = thymine
  modified_base 1936: mod_base = OTHER; note = thymine
  modified_base 1942: mod_base = OTHER; note = thymine
  modified_base 1951..1952: mod_base = OTHER; note = thymine
  modified_base 1955: mod_base = OTHER; note = thymine
  modified_base 1958: mod_base = OTHER; note = thymine
  modified_base 1960: mod_base = OTHER; note = thymine
  modified_base 1963: mod_base = OTHER; note = thymine
  modified_base 1968: mod_base = OTHER; note = thymine
  modified_base 1971..1972: mod_base = OTHER; note = thymine
  modified_base 1976: mod_base = OTHER; note = thymine
  modified_base 1979: mod_base = OTHER; note = thymine
  modified_base 1988: mod_base = OTHER; note = thymine
  modified_base 1990: mod_base = OTHER; note = thymine
  modified_base 1992: mod_base = OTHER; note = thymine
  modified_base 2009: mod_base = OTHER; note = thymine
  modified_base 2014: mod_base = OTHER; note = thymine
  modified_base 2018: mod_base = OTHER; note = thymine
  modified_base 2025: mod_base = OTHER; note = thymine
  modified_base 2028: mod_base = OTHER; note = thymine
  modified_base 2033..2034: mod_base = OTHER; note = thymine
  modified_base 2038: mod_base = OTHER; note = thymine
  modified_base 2040..2041: mod_base = OTHER; note = thymine
  modified_base 2043: mod_base = OTHER; note = thymine
  modified_base 2045: mod_base = OTHER; note = thymine
  modified_base 2047: mod_base = OTHER; note = thymine
  modified_base 2049..2051: mod_base = OTHER; note = thymine
  modified_base 2054..2055: mod_base = OTHER; note = thymine
  modified_base 2060: mod_base = OTHER; note = thymine
  modified_base 2065: mod_base = OTHER; note = thymine
  modified_base 2071: mod_base = OTHER; note = thymine
  modified_base 2073: mod_base = OTHER; note = thymine
  modified_base 2075: mod_base = OTHER; note = thymine
  modified_base 2078: mod_base = OTHER; note = thymine
  modified_base 2083..2087: mod_base = OTHER; note = thymine
  modified_base 2094..2096: mod_base = OTHER; note = thymine
  modified_base 2098: mod_base = OTHER; note = thymine
  modified_base 2101: mod_base = OTHER; note = thymine
  modified_base 2104: mod_base = OTHER; note = thymine
  modified_base 2106: mod_base = OTHER; note = thymine
  modified_base 2109..2112: mod_base = OTHER; note = thymine
  modified_base 2114: mod_base = OTHER; note = thymine
  modified_base 2122: mod_base = OTHER; note = thymine
  modified_base 2126: mod_base = OTHER; note = thymine
  modified_base 2128: mod_base = OTHER; note = thymine
  modified_base 2131: mod_base = OTHER; note = thymine
  modified_base 2134..2135: mod_base = OTHER; note = thymine
  modified_base 2139: mod_base = OTHER; note = thymine
  modified_base 2146: mod_base = OTHER; note = thymine
  modified_base 2159: mod_base = OTHER; note = thymine
  modified_base 2161: mod_base = OTHER; note = thymine
  modified_base 2170: mod_base = OTHER; note = thymine
  modified_base 2174: mod_base = OTHER; note = thymine
  modified_base 2178: mod_base = OTHER; note = thymine
  modified_base 2181: mod_base = OTHER; note = thymine
  modified_base 2184..2185: mod_base = OTHER; note = thymine
  modified_base 2189: mod_base = OTHER; note = thymine
  modified_base 2191: mod_base = OTHER; note = thymine
  modified_base 2204: mod_base = OTHER; note = thymine
  modified_base 2206..2207: mod_base = OTHER; note = thymine
  modified_base 2210: mod_base = OTHER; note = thymine
  modified_base 2215: mod_base = OTHER; note = thymine
  modified_base 2220..2222: mod_base = OTHER; note = thymine
  modified_base 2229: mod_base = OTHER; note = thymine
  modified_base 2233: mod_base = OTHER; note = thymine
  modified_base 2235: mod_base = OTHER; note = thymine
  modified_base 2237: mod_base = OTHER; note = thymine
  modified_base 2240: mod_base = OTHER; note = thymine
  modified_base 2247..2248: mod_base = OTHER; note = thymine
  modified_base 2250..2251: mod_base = OTHER; note = thymine
  modified_base 2253: mod_base = OTHER; note = thymine
  modified_base 2255: mod_base = OTHER; note = thymine
  modified_base 2257: mod_base = OTHER; note = thymine
  modified_base 2259: mod_base = OTHER; note = thymine
  modified_base 2261: mod_base = OTHER; note = thymine
  modified_base 2266..2267: mod_base = OTHER; note = thymine
  modified_base 2274: mod_base = OTHER; note = thymine
  modified_base 2276: mod_base = OTHER; note = thymine
  modified_base 2281..2282: mod_base = OTHER; note = thymine
  modified_base 2284: mod_base = OTHER; note = thymine
  modified_base 2290..2291: mod_base = OTHER; note = thymine
  modified_base 2293..2296: mod_base = OTHER; note = thymine
  modified_base 2301..2302: mod_base = OTHER; note = thymine
  modified_base 2304: mod_base = OTHER; note = thymine
  modified_base 2306: mod_base = OTHER; note = thymine
  modified_base 2310: mod_base = OTHER; note = thymine
  modified_base 2319: mod_base = OTHER; note = thymine
  modified_base 2322..2324: mod_base = OTHER; note = thymine
  modified_base 2327..2328: mod_base = OTHER; note = thymine
  modified_base 2330: mod_base = OTHER; note = thymine
  modified_base 2335: mod_base = OTHER; note = thymine
  modified_base 2339: mod_base = OTHER; note = thymine
  modified_base 2342..2343: mod_base = OTHER; note = thymine
  modified_base 2345: mod_base = OTHER; note = thymine
  modified_base 2347..2348: mod_base = OTHER; note = thymine
  modified_base 2350: mod_base = OTHER; note = thymine
  modified_base 2355: mod_base = OTHER; note = thymine
  modified_base 2360: mod_base = OTHER; note = thymine
  modified_base 2364: mod_base = OTHER; note = thymine
  modified_base 2366: mod_base = OTHER; note = thymine
  modified_base 2369..2371: mod_base = OTHER; note = thymine
  modified_base 2373: mod_base = OTHER; note = thymine
  modified_base 2376: mod_base = OTHER; note = thymine
  modified_base 2378: mod_base = OTHER; note = thymine
  modified_base 2387: mod_base = OTHER; note = thymine
  modified_base 2389: mod_base = OTHER; note = thymine
  modified_base 2401: mod_base = OTHER; note = thymine
  modified_base 2418..2420: mod_base = OTHER; note = thymine
  modified_base 2422: mod_base = OTHER; note = thymine
  modified_base 2431..2434: mod_base = OTHER; note = thymine
  modified_base 2437: mod_base = OTHER; note = thymine
  modified_base 2443..2445: mod_base = OTHER; note = thymine
  modified_base 2447: mod_base = OTHER; note = thymine
  modified_base 2449..2450: mod_base = OTHER; note = thymine
  modified_base 2454: mod_base = OTHER; note = thymine
  modified_base 2457..2459: mod_base = OTHER; note = thymine
  modified_base 2461: mod_base = OTHER; note = thymine
  modified_base 2464: mod_base = OTHER; note = thymine
  modified_base 2468: mod_base = OTHER; note = thymine
  modified_base 2474: mod_base = OTHER; note = thymine
  modified_base 2478..2479: mod_base = OTHER; note = thymine
  modified_base 2481: mod_base = OTHER; note = thymine
  modified_base 2483..2484: mod_base = OTHER; note = thymine
  modified_base 2487..2489: mod_base = OTHER; note = thymine
  modified_base 2494: mod_base = OTHER; note = thymine
  modified_base 2498: mod_base = OTHER; note = thymine
  modified_base 2501..2502: mod_base = OTHER; note = thymine
  modified_base 2505..2508: mod_base = OTHER; note = thymine
  modified_base 2510: mod_base = OTHER; note = thymine
  modified_base 2524..2525: mod_base = OTHER; note = thymine
  modified_base 2527: mod_base = OTHER; note = thymine
  modified_base 2529: mod_base = OTHER; note = thymine
  modified_base 2534: mod_base = OTHER; note = thymine
  modified_base 2538: mod_base = OTHER; note = thymine
  modified_base 2540: mod_base = OTHER; note = thymine
  modified_base 2543..2544: mod_base = OTHER; note = thymine
  modified_base 2546: mod_base = OTHER; note = thymine
  modified_base 2548: mod_base = OTHER; note = thymine
  modified_base 2555: mod_base = OTHER; note = thymine
  modified_base 2557..2559: mod_base = OTHER; note = thymine
  modified_base 2566: mod_base = OTHER; note = thymine
  modified_base 2573: mod_base = OTHER; note = thymine
  modified_base 2577: mod_base = OTHER; note = thymine
  modified_base 2587..2589: mod_base = OTHER; note = thymine
  modified_base 2592: mod_base = OTHER; note = thymine
  modified_base 2598: mod_base = OTHER; note = thymine
  modified_base 2606: mod_base = OTHER; note = thymine
  modified_base 2611..2612: mod_base = OTHER; note = thymine
  modified_base 2614: mod_base = OTHER; note = thymine
  modified_base 2624: mod_base = OTHER; note = thymine
  modified_base 2626..2627: mod_base = OTHER; note = thymine
  modified_base 2630: mod_base = OTHER; note = thymine
  modified_base 2636..2638: mod_base = OTHER; note = thymine
  modified_base 2641: mod_base = OTHER; note = thymine
  modified_base 2644..2645: mod_base = OTHER; note = thymine
  modified_base 2648: mod_base = OTHER; note = thymine
  modified_base 2654: mod_base = OTHER; note = thymine
  modified_base 2656: mod_base = OTHER; note = thymine
  modified_base 2665: mod_base = OTHER; note = thymine
  modified_base 2668: mod_base = OTHER; note = thymine
  modified_base 2670..2671: mod_base = OTHER; note = thymine
  modified_base 2674: mod_base = OTHER; note = thymine
  modified_base 2678: mod_base = OTHER; note = thymine
  modified_base 2682: mod_base = OTHER; note = thymine
  modified_base 2684: mod_base = OTHER; note = thymine
  modified_base 2686: mod_base = OTHER; note = thymine
  modified_base 2692: mod_base = OTHER; note = thymine
  modified_base 2696: mod_base = OTHER; note = thymine
  modified_base 2701..2703: mod_base = OTHER; note = thymine
  modified_base 2707: mod_base = OTHER; note = thymine
  modified_base 2710..2711: mod_base = OTHER; note = thymine
  modified_base 2717: mod_base = OTHER; note = thymine
  modified_base 2719: mod_base = OTHER; note = thymine
  modified_base 2721: mod_base = OTHER; note = thymine
  modified_base 2738..2739: mod_base = OTHER; note = thymine
  modified_base 2746: mod_base = OTHER; note = thymine
  modified_base 2748: mod_base = OTHER; note = thymine
  modified_base 2750: mod_base = OTHER; note = thymine
  modified_base 2756: mod_base = OTHER; note = thymine
  modified_base 2758..2759: mod_base = OTHER; note = thymine
  modified_base 2762..2763: mod_base = OTHER; note = thymine
  modified_base 2765: mod_base = OTHER; note = thymine
  modified_base 2767: mod_base = OTHER; note = thymine
  modified_base 2772: mod_base = OTHER; note = thymine
  modified_base 2775..2776: mod_base = OTHER; note = thymine
  modified_base 2783: mod_base = OTHER; note = thymine
  modified_base 2786: mod_base = OTHER; note = thymine
  modified_base 2790: mod_base = OTHER; note = thymine
  modified_base 2795..2796: mod_base = OTHER; note = thymine
  modified_base 2798: mod_base = OTHER; note = thymine
  modified_base 2802: mod_base = OTHER; note = thymine
  modified_base 2821: mod_base = OTHER; note = thymine
  modified_base 2824..2825: mod_base = OTHER; note = thymine
  modified_base 2827..2828: mod_base = OTHER; note = thymine
  modified_base 2832..2835: mod_base = OTHER; note = thymine
  modified_base 2842: mod_base = OTHER; note = thymine
  modified_base 2844..2846: mod_base = OTHER; note = thymine
  modified_base 2851: mod_base = OTHER; note = thymine
  modified_base 2856: mod_base = OTHER; note = thymine
  modified_base 2861: mod_base = OTHER; note = thymine
  modified_base 2872..2874: mod_base = OTHER; note = thymine
  modified_base 2882..2883: mod_base = OTHER; note = thymine
  modified_base 2886..2887: mod_base = OTHER; note = thymine
  modified_base 2890: mod_base = OTHER; note = thymine
  modified_base 2892..2893: mod_base = OTHER; note = thymine
  modified_base 2898: mod_base = OTHER; note = thymine
  modified_base 2904: mod_base = OTHER; note = thymine
  modified_base 2906..2907: mod_base = OTHER; note = thymine
  modified_base 2909: mod_base = OTHER; note = thymine
  modified_base 2913: mod_base = OTHER; note = thymine
  modified_base 2915: mod_base = OTHER; note = thymine
  modified_base 2917: mod_base = OTHER; note = thymine
  modified_base 2919: mod_base = OTHER; note = thymine
  modified_base 2922: mod_base = OTHER; note = thymine
  modified_base 2924: mod_base = OTHER; note = thymine
  modified_base 2926..2927: mod_base = OTHER; note = thymine
  modified_base 2934..2935: mod_base = OTHER; note = thymine
  modified_base 2937: mod_base = OTHER; note = thymine
  modified_base 2940..2941: mod_base = OTHER; note = thymine
  modified_base 2950..2951: mod_base = OTHER; note = thymine
  modified_base 2957: mod_base = OTHER; note = thymine
  modified_base 2959: mod_base = OTHER; note = thymine
  modified_base 2967..2968: mod_base = OTHER; note = thymine
  modified_base 2976: mod_base = OTHER; note = thymine
  modified_base 2978: mod_base = OTHER; note = thymine
  modified_base 2980: mod_base = OTHER; note = thymine
  modified_base 2982: mod_base = OTHER; note = thymine
  modified_base 2985: mod_base = OTHER; note = thymine
  modified_base 2987..2988: mod_base = OTHER; note = thymine
  modified_base 2990: mod_base = OTHER; note = thymine
  modified_base 2993: mod_base = OTHER; note = thymine
  modified_base 2995..2996: mod_base = OTHER; note = thymine
  modified_base 2998..3003: mod_base = OTHER; note = thymine
  modified_base 3006..3008: mod_base = OTHER; note = thymine
  modified_base 3010: mod_base = OTHER; note = thymine
  modified_base 3016: mod_base = OTHER; note = thymine
  modified_base 3028: mod_base = OTHER; note = thymine
  modified_base 3032: mod_base = OTHER; note = thymine
  modified_base 3036..3037: mod_base = OTHER; note = thymine
  modified_base 3040: mod_base = OTHER; note = thymine
  modified_base 3044: mod_base = OTHER; note = thymine
  modified_base 3052: mod_base = OTHER; note = thymine
  modified_base 3054: mod_base = OTHER; note = thymine
  modified_base 3061..3062: mod_base = OTHER; note = thymine
  modified_base 3069: mod_base = OTHER; note = thymine
  modified_base 3071: mod_base = OTHER; note = thymine
  modified_base 3073..3075: mod_base = OTHER; note = thymine
  modified_base 3078..3079: mod_base = OTHER; note = thymine
  modified_base 3086: mod_base = OTHER; note = thymine
  modified_base 3089: mod_base = OTHER; note = thymine
  modified_base 3092: mod_base = OTHER; note = thymine
  modified_base 3098: mod_base = OTHER; note = thymine
  modified_base 3102: mod_base = OTHER; note = thymine
  modified_base 3104: mod_base = OTHER; note = thymine
  modified_base 3106: mod_base = OTHER; note = thymine
  modified_base 3112: mod_base = OTHER; note = thymine
  modified_base 3114..3116: mod_base = OTHER; note = thymine
  modified_base 3118..3119: mod_base = OTHER; note = thymine
  modified_base 3124: mod_base = OTHER; note = thymine
  modified_base 3128: mod_base = OTHER; note = thymine
  modified_base 3133..3134: mod_base = OTHER; note = thymine
  modified_base 3136..3137: mod_base = OTHER; note = thymine
  modified_base 3142: mod_base = OTHER; note = thymine
  modified_base 3144..3145: mod_base = OTHER; note = thymine
  modified_base 3147..3148: mod_base = OTHER; note = thymine
  modified_base 3155..3158: mod_base = OTHER; note = thymine
  modified_base 3161: mod_base = OTHER; note = thymine
  modified_base 3163..3165: mod_base = OTHER; note = thymine
  modified_base 3167: mod_base = OTHER; note = thymine
  modified_base 3171: mod_base = OTHER; note = thymine
  modified_base 3178: mod_base = OTHER; note = thymine
  modified_base 3184: mod_base = OTHER; note = thymine
  modified_base 3186: mod_base = OTHER; note = thymine
  modified_base 3190: mod_base = OTHER; note = thymine
  modified_base 3192..3193: mod_base = OTHER; note = thymine
  modified_base 3196..3197: mod_base = OTHER; note = thymine
  modified_base 3202: mod_base = OTHER; note = thymine
  modified_base 3206: mod_base = OTHER; note = thymine
  modified_base 3209: mod_base = OTHER; note = thymine
  modified_base 3211: mod_base = OTHER; note = thymine
  modified_base 3218..3219: mod_base = OTHER; note = thymine
  modified_base 3223..3224: mod_base = OTHER; note = thymine
  modified_base 3229: mod_base = OTHER; note = thymine
  modified_base 3232..3233: mod_base = OTHER; note = thymine
  modified_base 3241: mod_base = OTHER; note = thymine
  modified_base 3247..3248: mod_base = OTHER; note = thymine
  modified_base 3254: mod_base = OTHER; note = thymine
  modified_base 3256: mod_base = OTHER; note = thymine
  modified_base 3260: mod_base = OTHER; note = thymine
  modified_base 3264..3265: mod_base = OTHER; note = thymine
  modified_base 3269: mod_base = OTHER; note = thymine
  modified_base 3276: mod_base = OTHER; note = thymine
  modified_base 3278: mod_base = OTHER; note = thymine
  modified_base 3281..3282: mod_base = OTHER; note = thymine
  modified_base 3289: mod_base = OTHER; note = thymine
  modified_base 3293: mod_base = OTHER; note = thymine
  modified_base 3297: mod_base = OTHER; note = thymine
  modified_base 3299: mod_base = OTHER; note = thymine
  modified_base 3305..3306: mod_base = OTHER; note = thymine
  modified_base 3313: mod_base = OTHER; note = thymine
  modified_base 3315..3317: mod_base = OTHER; note = thymine
  modified_base 3322: mod_base = OTHER; note = thymine
  modified_base 3324: mod_base = OTHER; note = thymine
  modified_base 3329: mod_base = OTHER; note = thymine
  modified_base 3338..3339: mod_base = OTHER; note = thymine
  modified_base 3341: mod_base = OTHER; note = thymine
  modified_base 3344: mod_base = OTHER; note = thymine
  modified_base 3347..3350: mod_base = OTHER; note = thymine
  modified_base 3353: mod_base = OTHER; note = thymine
  modified_base 3355: mod_base = OTHER; note = thymine
  modified_base 3360: mod_base = OTHER; note = thymine
  modified_base 3365..3366: mod_base = OTHER; note = thymine
  modified_base 3369: mod_base = OTHER; note = thymine
  modified_base 3373: mod_base = OTHER; note = thymine
  modified_base 3379: mod_base = OTHER; note = thymine
  modified_base 3381..3382: mod_base = OTHER; note = thymine
  modified_base 3389: mod_base = OTHER; note = thymine
  modified_base 3391: mod_base = OTHER; note = thymine
  modified_base 3395: mod_base = OTHER; note = thymine
  modified_base 3400: mod_base = OTHER; note = thymine
  modified_base 3405: mod_base = OTHER; note = thymine
  modified_base 3407: mod_base = OTHER; note = thymine
  misc_feature 1..3410: note = Junin virus strain Candid #1 segment S, completesequence.-ACCESSION FJ969442-VERSION FJ969442.1-GI:264665554 - Viruses; ssRNA viruses; ssRNA negative-strand viruses; Arenaviridae; Mammarenavirus.
  source 1..3410: mol_type = other RNA
cgcacagtggatcctaggcgattttggttacgctataattgtaactgttttctgtttggacaacatcaaaaacatccattgcacaatggggcagttcattagcttcatgcaagaaataccaacctttttgcaggaggctctgaacattgctcttgttgcagtcagtctcattgccatcattaagggtgtagtgaacctgtacaaaagtggtttgttccaattctttgtattcctagcactcgcaggaagatcctgcacagaagaagcttttaaaatcggactgcacacagagttccagactgtgtccttctcaatggtgggtctcttttccaacaatccacatgacctgcctctgttgtgtaccttaaacaagagccatctttacattaaggggggcaatgcttcatttcagatcagctttgatgatattgcagtattgttgccacagtatgatgttataatacaacatccagcagatatgagctggtgttccaaaagtgatgatcaaatttggttgtctcagtggttcatgaatgctgtgggacatgattggcatctagacccaccatttctgtgtaggaaccgtgcaaagacagaaggcttcatctttcaagtcaacacctccaagactggtgtcaatggaaattatgctaagaagtttaagactggcatgcatcatttatatagagaatatcctgacccttgcttgaatggcaaactgtgcttaatgaaggcacaacctaccagttggcctctccaatgtccactcgaccacgttaacacattacacttccttacaagaggtaaaaacattcaacttccaaggaggtccttgaaagcattcttctcctggtctttgacagactcatccggcaaggatacccctggaggctattgtctagaagagtggatgctcgttgcagccaaaatgaagtgttttggcaatactgctgtagcaaaatgcaatctgaatcatgactctgaattctgtgacatgctgaggctttttgattacaacaaaaatgctatcaaaaccttaaatgatgaaactaagaaacaagtaaatctgatgggacagacaatcaatgcgctgatatctgacaatttattgatgaaaaacaaaattagggaactgatgagtgtcccttactgcaattacacaaaattttggtatgtcaaccacacactttcaggacaacactcattaccaaggtgctggttaataaaaaacaacagctatttgaacatctctgacttccgtaatgactggatattagaaagtgacttcttaatttctgaaatgctaagcaaagagtattcggacaggcagggtaaaactcctttgactttagttgacatctgtatttggagcacagtattcttcacagcgtcactcttccttcacttggtgggtataccctcccacagacacatcaggggcgaagcatgccctttgccacacaggttgaacagcttgggtggttgcagatgtggtaagtaccccaatctaaagaaaccaacagtttggcgtagaggacactaagacctcctgagggtccccaccagcccgggcactgcccgggctggtgtggccccccagtccgcggcctggccgcggactggggaggcactgcttacagtgcataggctgccttcgggaggaacagcaagctcggtggtaatagaggtgtaggttcctcctcatagagcttcccatctagcactgactgaaacattatgcagtctagcagagcacagtgtggttcactggaggccaacttgaagggagtatccttttccctctttttcttattgacaaccactccattgtgatatttgcataagtgaccatatttctcccagacctgttgatcaaactgcctggcttgttcagatgtgagcttaacatcaaccagtttaagatctcttcttccatggaggtcaaacaacttcctgatgtcatcggatccttgagtagtcacaaccatgtctggaggcagcaagccgatcacgtaactaagaactcctggcattgcatcttctatgtctttcattaagatgccgtgagagtgtctgctaccatttttaaaccctttctcatcatgtggttttctgaagcagtgaatatactgcttacctgcaggctggaacaacgccatctcaacagggtcagtagctggtccttcaatgtcgagccaaagggtattggtggggtcgagtttccccactgcctctctgatgacagcttcttgtatctctgtcaagttagccaatctcaaattctgaccgttcttttccggttgtctaggtccagcaactggtttccttgtcagatcaatacttgtgttgtcccatgacctgcctatgatttgtgatctggaaccaatataaggccaaccatcgccagaaaggcaaagtttgtacagaaggttttcataagggtttctattgcctggtttctcatcaataaacatgccttctcttcgtttaacctgaatggttgattttatgagggaagaaaagttatctggggtgactctgattgtctccaacatatttccaccatcaagaatggatgcaccagcctttactgcagctgaaagactaaagttgtagccagaaatgttgatggagctttcatccttagtcacaatctggaggcagtcatgttcctgagtcaatctgtcaaggtcactcaagtttggatacttcacagtgtatagaagcccaagagaggttaaagcctgtatgacactgttcattgtctcacctccttgaacagtcatgcatgcaattgtcaatgcaggaacagaaccaaactgattgttaagttttgaaggatctttaacatcccataccctcaccacaccatttcccccagttccttgctgttgaaatcccagtgttctcaatatctctgatctcttggccagttgtgattgagacaagttacccatgtaaaccccttgagagcctgtctctgctcttctaatcttgttttttaatttctcaaggtcagacgccaactccatcagttcatccctccccagatctcccaccttgaaaactgtgtttcgttgaacactcctcatggacatgagtctgtcaacctctttattcaggtccctcaacttattgaggtcttcttccccccttttagtctttctgagtgcccgctgcacctgtgcctcttggttgaagtcaatgctgtcagcaattagcttggcatccttcagaacatccgacttgacagtctgagtaaattgactcaaacctctccttaaggactgagtccatctaaagcttggaacctctttggagtgtgccatgccagaagttctggtgattttgatctagaatagagttgctcagtgaaagtgttagacactatgcctaggatccactgtgcg

SEQ ID NO: 4 - RNA, 7114 nt, unidentified; identical to SEQ ID NO: 2
  modified_base 8: mod_base = OTHER; note = thymine
  modified_base 12: mod_base = OTHER; note = thymine
  modified_base 15: mod_base = OTHER; note = thymine
  modified_base 21: mod_base = OTHER; note = thymine
  modified_base 25..26: mod_base = OTHER; note = thymine
  modified_base 29: mod_base = OTHER; note = thymine
  modified_base 32..33: mod_base = OTHER; note = thymine
  modified_base 38: mod_base = OTHER; note = thymine
  modified_base 40: mod_base = OTHER; note = thymine
  modified_base 45..46: mod_base = OTHER; note = thymine
  modified_base 48: mod_base = OTHER; note = thymine
  modified_base 51: mod_base = OTHER; note = thymine
  modified_base 53: mod_base = OTHER; note = thymine
  modified_base 57: mod_base = OTHER; note = thymine
  modified_base 59: mod_base = OTHER; note = thymine
  modified_base 63..64: mod_base = OTHER; note = thymine
  modified_base 67: mod_base = OTHER; note = thymine
  modified_base 85: mod_base = OTHER; note = thymine
  modified_base 93: mod_base = OTHER; note = thymine
  modified_base 105: mod_base = OTHER; note = thymine
  modified_base 111: mod_base = OTHER; note = thymine
  modified_base 113: mod_base = OTHER; note = thymine
  modified_base 126: mod_base = OTHER; note = thymine
  modified_base 129: mod_base = OTHER; note = thymine
  modified_base 156..158: mod_base = OTHER; note = thymine
  modified_base 166: mod_base = OTHER; note = thymine
  modified_base 170: mod_base = OTHER; note = thymine
  modified_base 179: mod_base = OTHER; note = thymine
  modified_base 181: mod_base = OTHER; note = thymine
  modified_base 183: mod_base = OTHER; note = thymine
  modified_base 185: mod_base = OTHER; note = thymine
  modified_base 188: mod_base = OTHER; note = thymine
  modified_base 192: mod_base = OTHER; note = thymine
  modified_base 194: mod_base = OTHER; note = thymine
  modified_base 198: mod_base = OTHER; note = thymine
  modified_base 200: mod_base = OTHER; note = thymine
  modified_base 204: mod_base = OTHER; note = thymine
  modified_base 207: mod_base = OTHER; note = thymine
  modified_base 210: mod_base = OTHER; note = thymine
  modified_base 213..215: mod_base = OTHER; note = thymine
  modified_base 218: mod_base = OTHER; note = thymine
  modified_base 221: mod_base = OTHER; note = thymine
  modified_base 227..229: mod_base = OTHER; note = thymine
  modified_base 232: mod_base = OTHER; note = thymine
  modified_base 237: mod_base = OTHER; note = thymine
  modified_base 239: mod_base = OTHER; note = thymine
  modified_base 242: mod_base = OTHER; note = thymine
  modified_base 245: mod_base = OTHER; note = thymine
  modified_base 249: mod_base = OTHER; note = thymine
  modified_base 253..255: mod_base = OTHER; note = thymine
  modified_base 257..259: mod_base = OTHER; note = thymine
  modified_base 264: mod_base = OTHER; note = thymine
  modified_base 269: mod_base = OTHER; note = thymine
  modified_base 275: mod_base = OTHER; note = thymine
  modified_base 277: mod_base = OTHER; note = thymine
  modified_base 279..280: mod_base = OTHER; note = thymine
  modified_base 287..288: mod_base = OTHER; note = thymine
  modified_base 293: mod_base = OTHER; note = thymine
  modified_base 295: mod_base = OTHER; note = thymine
  modified_base 297: mod_base = OTHER; note = thymine
  modified_base 302: mod_base = OTHER; note = thymine
  modified_base 304: mod_base = OTHER; note = thymine
  modified_base 306: mod_base = OTHER; note = thymine
  modified_base 309: mod_base = OTHER; note = thymine
  modified_base 319: mod_base = OTHER; note = thymine
  modified_base 331: mod_base = OTHER; note = thymine
  modified_base 337: mod_base = OTHER; note = thymine
  modified_base 343: mod_base = OTHER; note = thymine
  modified_base 366: mod_base = OTHER; note = thymine
  modified_base 375: mod_base = OTHER; note = thymine
  modified_base 384: mod_base = OTHER; note = thymine
  modified_base 412: mod_base = OTHER; note = thymine
  modified_base 425: mod_base = OTHER; note = thymine
  modified_base 442: mod_base = OTHER; note = thymine
  modified_base 445: mod_base = OTHER; note = thymine
  modified_base 452..453: mod_base = OTHER; note = thymine
  modified_base 456: mod_base = OTHER; note = thymine
  modified_base 460: mod_base = OTHER; note = thymine
  modified_base 462: mod_base = OTHER; note = thymine
  modified_base 464..466: mod_base = OTHER; note = thymine
  modified_base 469: mod_base = OTHER; note = thymine
  modified_base 471: mod_base = OTHER; note = thymine
  modified_base 476..478: mod_base = OTHER; note = thymine
  modified_base 480: mod_base = OTHER; note = thymine
  modified_base 483..484: mod_base = OTHER; note = thymine
  modified_base 488: mod_base = OTHER; note = thymine
  modified_base 493: mod_base = OTHER; note = thymine
  modified_base 497: mod_base = OTHER; note = thymine
  modified_base 499: mod_base = OTHER; note = thymine
  modified_base 506..508: mod_base = OTHER; note = thymine
  modified_base 512: mod_base = OTHER; note = thymine
  modified_base 514: mod_base = OTHER; note = thymine
  modified_base 519: mod_base = OTHER; note = thymine
  modified_base 524..525: mod_base = OTHER; note = thymine
  modified_base 531: mod_base = OTHER; note = thymine
  modified_base 533: mod_base = OTHER; note = thymine
  modified_base 536: mod_base = OTHER; note = thymine
  modified_base 538: mod_base = OTHER; note = thymine
  modified_base 546: mod_base = OTHER; note = thymine
  modified_base 549..551: mod_base = OTHER; note = thymine
  modified_base 557: mod_base = OTHER; note = thymine
  modified_base 559: mod_base = OTHER; note = thymine
  modified_base 562: mod_base = OTHER; note = thymine
  modified_base 566: mod_base = OTHER; note = thymine
  modified_base 573..574: mod_base = OTHER; note = thymine
  modified_base 579..581: mod_base = OTHER; note = thymine
  modified_base 587..588: mod_base = OTHER; note = thymine
  modified_base 597: mod_base = OTHER; note = thymine
  modified_base 605..607: mod_base = OTHER; note = thymine
  modified_base 609: mod_base = OTHER; note = thymine
  modified_base 614: mod_base = OTHER; note = thymine
  modified_base 622: mod_base = OTHER; note = thymine
  modified_base 627: mod_base = OTHER; note = thymine
  modified_base 630: mod_base = OTHER; note = thymine
  modified_base 632..634: mod_base = OTHER; note = thymine
  modified_base 636: mod_base = OTHER; note = thymine
  modified_base 638: mod_base = OTHER; note = thymine
  modified_base 642: mod_base = OTHER; note = thymine
  modified_base 645: mod_base = OTHER; note = thymine
  modified_base 660: mod_base = OTHER; note = thymine
  modified_base 663: mod_base = OTHER; note = thymine
  modified_base 665..666: mod_base = OTHER; note = thymine
  modified_base 669: mod_base = OTHER; note = thymine
  modified_base 674: mod_base = OTHER; note = thymine
  modified_base 677: mod_base = OTHER; note = thymine
  modified_base 679..680: mod_base = OTHER; note = thymine
  modified_base 682..684: mod_base = OTHER; note = thymine
  modified_base 686: mod_base = OTHER; note = thymine
  modified_base 689: mod_base = OTHER; note = thymine
  modified_base 691..692: mod_base = OTHER; note = thymine
  modified_base 694..698: mod_base = OTHER; note = thymine
  modified_base 702: mod_base = OTHER; note = thymine
  modified_base 710: mod_base = OTHER; note = thymine
  modified_base 713..714: mod_base = OTHER; note = thymine
  modified_base 718..719: mod_base = OTHER; note = thymine
  modified_base 722: mod_base = OTHER; note = thymine
  modified_base 727..729: mod_base = OTHER; note = thymine
  modified_base 736: mod_base = OTHER; note = thymine
  modified_base 738: mod_base = OTHER; note = thymine
  modified_base 742: mod_base = OTHER; note = thymine
  modified_base 744: mod_base = OTHER; note = thymine
  modified_base 747: mod_base = OTHER; note = thymine
  modified_base 749: mod_base = OTHER; note = thymine
  modified_base 752: mod_base = OTHER; note = thymine
  modified_base 755: mod_base = OTHER; note = thymine
  modified_base 760..761: mod_base = OTHER; note = thymine
  modified_base 765..766: mod_base = OTHER; note = thymine
  modified_base 770: mod_base = OTHER; note = thymine
  modified_base 772: mod_base = OTHER; note = thymine
  modified_base 777: mod_base = OTHER; note = thymine
  modified_base 789: mod_base = OTHER; note = thymine
  modified_base 794: mod_base = OTHER; note = thymine
  modified_base 796: mod_base = OTHER; note = thymine
  modified_base 798: mod_base = OTHER; note = thymine
  modified_base 800: mod_base = OTHER; note = thymine
  modified_base 802..803: mod_base = OTHER; note = thymine
  modified_base 811..813: mod_base = OTHER; note = thymine
  modified_base 816..817: mod_base = OTHER; note = thymine
  modified_base 821: mod_base = OTHER; note = thymine
  modified_base 823: mod_base = OTHER; note = thymine
  modified_base 825: mod_base = OTHER; note = thymine
  modified_base 832: mod_base = OTHER; note = thymine
  modified_base 837: mod_base = OTHER; note = thymine
  modified_base 839..841: mod_base = OTHER; note = thymine
  modified_base 854..855: mod_base = OTHER; note = thymine
  modified_base 857: mod_base = OTHER; note = thymine
  modified_base 860: mod_base = OTHER; note = thymine
  modified_base 864..865: mod_base = OTHER; note = thymine
  modified_base 867: mod_base = OTHER; note = thymine
  modified_base 869: mod_base = OTHER; note = thymine
  modified_base 877: mod_base = OTHER; note = thymine
  modified_base 882: mod_base = OTHER; note = thymine
  modified_base 885: mod_base = OTHER; note = thymine
  modified_base 887: mod_base = OTHER; note = thymine
  modified_base 891: mod_base = OTHER; note = thymine
  modified_base 894..895: mod_base = OTHER; note = thymine
  modified_base 897..898: mod_base = OTHER; note = thymine
  modified_base 901..902: mod_base = OTHER; note = thymine
  modified_base 905..906: mod_base = OTHER; note = thymine
  modified_base 912: mod_base = OTHER; note = thymine
  modified_base 918: mod_base = OTHER; note = thymine
  modified_base 927..928: mod_base = OTHER; note = thymine
  modified_base 933..934: mod_base = OTHER; note = thymine
  modified_base 939: mod_base = OTHER; note = thymine
  modified_base 943: mod_base = OTHER; note = thymine
  modified_base 949: mod_base = OTHER; note = thymine
  modified_base 951..952: mod_base = OTHER; note = thymine
  modified_base 954: mod_base = OTHER; note = thymine
  modified_base 957: mod_base = OTHER; note = thymine
  modified_base 960..962: mod_base = OTHER; note = thymine
  modified_base 964: mod_base = OTHER; note = thymine
  modified_base 972: mod_base = OTHER; note = thymine
  modified_base 977: mod_base = OTHER; note = thymine
  modified_base 981: mod_base = OTHER; note = thymine
  modified_base 985: mod_base = OTHER; note = thymine
  modified_base 998: mod_base = OTHER; note = thymine
  modified_base 1002: mod_base = OTHER; note = thymine
  modified_base 1009: mod_base = OTHER; note = thymine
  modified_base 1011: mod_base = OTHER; note = thymine
  modified_base 1014: mod_base = OTHER; note = thymine
  modified_base 1019: mod_base = OTHER; note = thymine
  modified_base 1021: mod_base = OTHER; note = thymine
  modified_base 1028: mod_base = OTHER; note = thymine
  modified_base 1031: mod_base = OTHER; note = thymine
  modified_base 1036: mod_base = OTHER; note = thymine
  modified_base 1042: mod_base = OTHER; note = thymine
  modified_base 1049: mod_base = OTHER; note = thymine
  modified_base 1056: mod_base = OTHER; note = thymine
  modified_base 1064: mod_base = OTHER; note = thymine
  modified_base 1068: mod_base = OTHER; note = thymine
  modified_base 1072..1073: mod_base = OTHER; note = thymine
  modified_base 1077: mod_base = OTHER; note = thymine
  modified_base 1094..1095: mod_base = OTHER; note = thymine
  modified_base 1099: mod_base = OTHER; note = thymine
  modified_base 1102..1103: mod_base = OTHER; note = thymine
  modified_base 1107: mod_base = OTHER; note = thymine
  modified_base 1113..1114: mod_base = OTHER; note = thymine
  modified_base 1119: mod_base = OTHER; note = thymine
  modified_base 1121: mod_base = OTHER; note = thymine
  modified_base 1124..1125: mod_base = OTHER; note = thymine
  modified_base 1132: mod_base = OTHER; note = thymine
  modified_base 1140: mod_base = OTHER; note = thymine
  modified_base 1143: mod_base = OTHER; note = thymine
  modified_base 1148: mod_base = OTHER; note = thymine
  modified_base 1151: mod_base = OTHER; note = thymine
  modified_base 1154: mod_base = OTHER; note = thymine
  modified_base 1159: mod_base = OTHER; note = thymine
  modified_base 1165: mod_base = OTHER; note = thymine
  modified_base 1168: mod_base = OTHER; note = thymine
  modified_base 1170: mod_base = OTHER; note = thymine
  modified_base 1174: mod_base = OTHER; note = thymine
  modified_base 1176: mod_base = OTHER; note = thymine
  modified_base 1183: mod_base = OTHER; note = thymine
  modified_base 1185..1187: mod_base = OTHER; note = thymine
  modified_base 1189..1190: mod_base = OTHER; note = thymine
  modified_base 1192: mod_base = OTHER; note = thymine
  modified_base 1196..1198: mod_base = OTHER; note = thymine
  modified_base 1202..1205: mod_base = OTHER; note = thymine
  modified_base 1207: mod_base = OTHER; note = thymine
  modified_base 1214: mod_base = OTHER; note = thymine
  modified_base 1217: mod_base = OTHER; note = thymine
  modified_base 1219: mod_base = OTHER; note = thymine
  modified_base 1221: mod_base = OTHER; note = thymine
  modified_base 1223..1224: mod_base = OTHER; note = thymine
  modified_base 1226: mod_base = OTHER; note = thymine
  modified_base 1229..1230: mod_base = OTHER; note = thymine
  modified_base 1234: mod_base = OTHER; note = thymine
  modified_base 1237: mod_base = OTHER; note = thymine
  modified_base 1241..1244: mod_base = OTHER; note = thymine
  modified_base 1247..1249: mod_base = OTHER; note = thymine
  modified_base 1256..1257: mod_base = OTHER; note = thymine
  modified_base 1259..1260: mod_base = OTHER; note = thymine
  modified_base 1272..1273: mod_base = OTHER; note = thymine
  modified_base 1285: mod_base = OTHER; note = thymine
  modified_base 1287: mod_base = OTHER; note = thymine
  modified_base 1293: mod_base = OTHER; note = thymine
  modified_base 1297: mod_base = OTHER; note = thymine
  modified_base 1300: mod_base = OTHER; note = thymine
  modified_base 1302: mod_base = OTHER; note = thymine
  modified_base 1304..1305: mod_base = OTHER; note = thymine
  modified_base 1308: mod_base = OTHER; note = thymine
  modified_base 1324: mod_base = OTHER; note = thymine
  modified_base 1326..1327: mod_base = OTHER; note = thymine
  modified_base 1329: mod_base = OTHER; note = thymine
  modified_base 1344: mod_base = OTHER; note = thymine
  modified_base 1356..1357: mod_base = OTHER; note = thymine
  modified_base 1360: mod_base = OTHER; note = thymine
  modified_base 1362..1363: mod_base = OTHER; note = thymine
  modified_base 1365: mod_base = OTHER; note = thymine
  modified_base 1372: mod_base = OTHER; note = thymine
  modified_base 1379: mod_base = OTHER; note = thymine
  modified_base 1383: mod_base = OTHER; note = thymine
  modified_base 1390..1391: mod_base = OTHER; note = thymine
  modified_base 1396: mod_base = OTHER; note = thymine
  modified_base 1398: mod_base = OTHER; note = thymine
  modified_base 1400..1401: mod_base = OTHER; note = thymine
  modified_base 1411..1412: mod_base = OTHER; note = thymine
  modified_base 1415: mod_base = OTHER; note = thymine
  modified_base 1417..1418: mod_base = OTHER; note = thymine
  modified_base 1421: mod_base = OTHER; note = thymine
  modified_base 1424..1426: mod_base = OTHER; note = thymine
  modified_base 1428..1429: mod_base = OTHER; note = thymine
  modified_base 1431..1432: mod_base = OTHER; note = thymine
  modified_base 1435: mod_base = OTHER; note = thymine
  modified_base 1439: mod_base = OTHER; note = thymine
  modified_base 1447: mod_base = OTHER; note = thymine
  modified_base 1452: mod_base = OTHER; note = thymine
  modified_base 1454: mod_base = OTHER; note = thymine
  modified_base 1456: mod_base = OTHER; note = thymine
  modified_base 1460..1461: mod_base = OTHER; note = thymine
  modified_base 1466: mod_base = OTHER; note = thymine
  modified_base 1470: mod_base = OTHER; note = thymine
  modified_base 1472: mod_base = OTHER; note = thymine
  modified_base 1475..1476: mod_base = OTHER; note = thymine
  modified_base 1480: mod_base = OTHER; note = thymine
  modified_base 1482: mod_base = OTHER; note = thymine
  modified_base 1485: mod_base = OTHER; note = thymine
  modified_base 1487..1488: mod_base = OTHER; note = thymine
  modified_base 1490: mod_base = OTHER; note = thymine
  modified_base 1494: mod_base = OTHER; note = thymine
  modified_base 1496: mod_base = OTHER; note = thymine
  modified_base 1502..1503: mod_base = OTHER; note = thymine
  modified_base 1506..1507: mod_base = OTHER; note = thymine
  modified_base 1523..1525: mod_base = OTHER; note = thymine
  modified_base 1535: mod_base = OTHER; note = thymine
  modified_base 1537..1538: mod_base = OTHER; note = thymine
  modified_base 1543: mod_base = OTHER; note = thymine
  modified_base 1547: mod_base = OTHER; note = thymine
  modified_base 1550: mod_base = OTHER; note = thymine
  modified_base 1554..1556: mod_base = OTHER; note = thymine
  modified_base 1558: mod_base = OTHER; note = thymine
  modified_base 1561: mod_base = OTHER; note = thymine
  modified_base 1568: mod_base = OTHER; note = thymine
  modified_base 1577..1578: mod_base = OTHER; note = thymine
  modified_base 1585: mod_base = OTHER; note = thymine
  modified_base 1590..1591: mod_base = OTHER; note = thymine
  modified_base 1594: mod_base = OTHER; note = thymine
  modified_base 1601..1602: mod_base = OTHER; note = thymine
  modified_base 1605: mod_base = OTHER; note = thymine
  modified_base 1607: mod_base = OTHER; note = thymine
  modified_base 1612: mod_base = OTHER; note = thymine
  modified_base 1615: mod_base = OTHER; note = thymine
  modified_base 1622..1623: mod_base = OTHER; note = thymine
  modified_base 1629..1631: mod_base = OTHER; note = thymine
  modified_base 1633: mod_base = OTHER; note = thymine
  modified_base 1635: mod_base = OTHER; note = thymine
  modified_base 1638: mod_base = OTHER; note = thymine
  modified_base 1640: mod_base = OTHER; note = thymine
  modified_base 1644: mod_base = OTHER; note = thymine
  modified_base 1646: mod_base = OTHER; note = thymine
  modified_base 1648: mod_base = OTHER; note = thymine
  modified_base 1653: mod_base = OTHER; note = thymine
  modified_base 1658: mod_base = OTHER; note = thymine
  modified_base 1668: mod_base = OTHER; note = thymine
  modified_base 1672: mod_base = OTHER; note = thymine
  modified_base 1674: mod_base = OTHER; note = thymine
  modified_base 1676..1678: mod_base = OTHER; note = thymine
  modified_base 1684: mod_base = OTHER; note = thymine
  modified_base 1686: mod_base = OTHER; note = thymine
  modified_base 1692..1693: mod_base = OTHER; note = thymine
  modified_base 1695..1696: mod_base = OTHER; note = thymine
  modified_base 1698: mod_base = OTHER; note = thymine
  modified_base 1700: mod_base = OTHER; note = thymine
  modified_base 1702..1703: mod_base = OTHER; note = thymine
  modified_base 1706..1708: mod_base = OTHER; note = thymine
  modified_base 1712: mod_base = OTHER; note = thymine
  modified_base 1716: mod_base = OTHER; note = thymine
  modified_base 1720..1721: mod_base = OTHER; note = thymine
  modified_base 1723: mod_base = OTHER; note = thymine
  modified_base 1726: mod_base = OTHER; note = thymine
  modified_base 1730: mod_base = OTHER; note = thymine
  modified_base 1734..1737: mod_base = OTHER; note = thymine
  modified_base 1740..1741: mod_base = OTHER; note = thymine
  modified_base 1744: mod_base = OTHER; note = thymine
  modified_base 1747: mod_base = OTHER; note = thymine
  modified_base 1749: mod_base = OTHER; note = thymine
  modified_base 1751: mod_base = OTHER; note = thymine
  modified_base 1767: mod_base = OTHER; note = thymine
  modified_base 1769: mod_base = OTHER; note = thymine
  modified_base 1776..1777: mod_base = OTHER; note = thymine
  modified_base 1781: mod_base = OTHER; note = thymine
  modified_base 1783..1784: mod_base = OTHER; note = thymine
  modified_base 1789: mod_base = OTHER; note = thymine
  modified_base 1791: mod_base = OTHER; note = thymine
  modified_base 1799: mod_base = OTHER; note = thymine
  modified_base 1801: mod_base = OTHER; note = thymine
  modified_base 1803..1804: mod_base = OTHER; note = thymine
  modified_base 1808..1809: mod_base = OTHER; note = thymine
  modified_base 1815: mod_base = OTHER; note = thymine
  modified_base 1818..1819: mod_base = OTHER; note = thymine
  modified_base 1822: mod_base = OTHER; note = thymine
  modified_base 1829: mod_base = OTHER; note = thymine
  modified_base 1836: mod_base = OTHER; note = thymine
  modified_base 1838..1839: mod_base = OTHER; note = thymine
  modified_base 1841..1842: mod_base = OTHER; note = thymine
  modified_base 1847: mod_base = OTHER; note = thymine
  modified_base 1850..1852: mod_base = OTHER; note = thymine
  modified_base 1857: mod_base = OTHER; note = thymine
  modified_base 1860..1862: mod_base = OTHER; note = thymine
  modified_base 1864..1865: mod_base = OTHER; note = thymine
  modified_base 1868: mod_base = OTHER; note = thymine
  modified_base 1870: mod_base = OTHER; note = thymine
  modified_base 1875: mod_base = OTHER; note = thymine
  modified_base 1880: mod_base = OTHER; note = thymine
  modified_base 1884: mod_base = OTHER; note = thymine
  modified_base 1887..1888: mod_base = OTHER; note = thymine
  modified_base 1891: mod_base = OTHER; note = thymine
  modified_base 1895: mod_base = OTHER; note = thymine
  modified_base 1898..1899: mod_base = OTHER; note = thymine
  modified_base 1902: mod_base = OTHER; note = thymine
  modified_base 1904..1905: mod_base = OTHER; note = thymine
  modified_base 1907: mod_base = OTHER; note = thymine
  modified_base 1909: mod_base = OTHER; note = thymine
  modified_base 1911: mod_base = OTHER; note = thymine
  modified_base 1913: mod_base = OTHER; note = thymine
  modified_base 1916: mod_base = OTHER; note = thymine
  modified_base 1919: mod_base = OTHER; note = thymine
  modified_base 1922..1924: mod_base = OTHER; note = thymine
  modified_base 1928..1930: mod_base = OTHER; note = thymine
  modified_base 1947..1948: mod_base = OTHER; note = thymine
  modified_base 1950..1951: mod_base = OTHER; note = thymine
  modified_base 1953..1954: mod_base = OTHER; note = thymine
  modified_base 1958: mod_base = OTHER; note = thymine
  modified_base 1974: mod_base = OTHER; note = thymine
  modified_base 1976: mod_base = OTHER; note = thymine
  modified_base 1981: mod_base = OTHER; note = thymine
  modified_base 1983..1984: mod_base = OTHER; note = thymine
  modified_base 1987: mod_base = OTHER; note = thymine
  modified_base 2004: mod_base = OTHER; note = thymine
  modified_base 2009: mod_base = OTHER; note = thymine
  modified_base 2013: mod_base = OTHER; note = thymine
  modified_base 2022..2024: mod_base = OTHER; note = thymine
  modified_base 2026: mod_base = OTHER; note = thymine
  modified_base 2043: mod_base = OTHER; note = thymine
  modified_base 2046: mod_base = OTHER; note = thymine
  modified_base 2056..2057: mod_base = OTHER; note = thymine
  modified_base 2060..2062: mod_base = OTHER; note = thymine
  modified_base 2069..2070: mod_base = OTHER; note = thymine
  modified_base 2073..2074: mod_base = OTHER; note = thymine
  modified_base 2078..2079: mod_base = OTHER; note = thymine
  modified_base 2081..2083: mod_base = OTHER; note = thymine
  modified_base 2087: mod_base = OTHER; note = thymine
  modified_base 2090..2094: mod_base = OTHER; note = thymine
  modified_base 2100: mod_base = OTHER; note = thymine
  modified_base 2104: mod_base = OTHER; note = thymine
  modified_base 2108..2110: mod_base = OTHER; note = thymine
  modified_base 2115..2117: mod_base = OTHER; note = thymine
  modified_base 2119: mod_base = OTHER; note = thymine
  modified_base 2121: mod_base = OTHER; note = thymine
  modified_base 2128..2129: mod_base = OTHER; note = thymine
  modified_base 2141: mod_base = OTHER; note = thymine
  modified_base 2146..2147: mod_base = OTHER; note = thymine
  modified_base 2149..2150: mod_base = OTHER; note = thymine
  modified_base 2155: mod_base = OTHER; note = thymine
  modified_base 2159: mod_base = OTHER; note = thymine
  modified_base 2161: mod_base = OTHER; note = thymine
  modified_base 2163: mod_base = OTHER; note = thymine
  modified_base 2166..2168: mod_base = OTHER; note = thymine
  modified_base 2170: mod_base = OTHER; note = thymine
  modified_base 2172: mod_base = OTHER; note = thymine
  modified_base 2180: mod_base = OTHER; note = thymine
  modified_base 2186..2188: mod_base = OTHER; note = thymine
  modified_base 2193: mod_base = OTHER; note = thymine
  modified_base 2197: mod_base = OTHER; note = thymine
  modified_base 2205..2206: mod_base = OTHER; note = thymine
  modified_base 2209: mod_base = OTHER; note = thymine
  modified_base 2215: mod_base = OTHER; note = thymine
  modified_base 2218: mod_base = OTHER; note = thymine
  modified_base 2220..2221: mod_base = OTHER; note = thymine
  modified_base 2233: mod_base = OTHER; note = thymine
  modified_base 2240: mod_base = OTHER; note = thymine
  modified_base 2256: mod_base = OTHER; note = thymine
  modified_base 2261: mod_base = OTHER; note = thymine
  modified_base 2267..2269: mod_base = OTHER; note = thymine
  modified_base 2271..2272: mod_base = OTHER; note = thymine
  modified_base 2275..2276: mod_base = OTHER; note = thymine
  modified_base 2280: mod_base = OTHER; note = thymine
  modified_base 2282: mod_base = OTHER; note = thymine
  modified_base 2290: mod_base = OTHER; note = thymine
  modified_base 2292..2293: mod_base = OTHER; note = thymine
  modified_base 2295..2296: mod_base = OTHER; note = thymine
  modified_base 2303..2306: mod_base = OTHER; note = thymine
  modified_base 2308: mod_base = OTHER; note = thymine
  modified_base 2313..2315: mod_base = OTHER; note = thymine
  modified_base 2317..2318: mod_base = OTHER; note = thymine
  modified_base 2326: mod_base = OTHER; note = thymine
  modified_base 2331: mod_base = OTHER; note = thymine
  modified_base 2333: mod_base = OTHER; note = thymine
  modified_base 2335..2336: mod_base = OTHER; note = thymine
  modified_base 2338: mod_base = OTHER; note = thymine
  modified_base 2341..2342: mod_base = OTHER; note = thymine
  modified_base 2344: mod_base = OTHER; note = thymine
  modified_base 2348: mod_base = OTHER; note = thymine
  modified_base 2350..2351: mod_base = OTHER; note = thymine
  modified_base 2356: mod_base = OTHER; note = thymine
  modified_base 2358: mod_base = OTHER; note = thymine
  modified_base 2360..2361: mod_base = OTHER; note = thymine
  modified_base 2363..2365: mod_base = OTHER; note = thymine
  modified_base 2367: mod_base = OTHER; note = thymine
  modified_base 2369: mod_base = OTHER; note = thymine
  modified_base 2375: mod_base = OTHER; note = thymine
  modified_base 2377..2378: mod_base = OTHER; note = thymine
  modified_base 2383..2384: mod_base = OTHER; note = thymine
  modified_base 2386: mod_base = OTHER; note = thymine
  modified_base 2389: mod_base = OTHER; note = thymine
  modified_base 2393..2395: mod_base = OTHER; note = thymine
  modified_base 2398: mod_base = OTHER; note = thymine
  modified_base 2401: mod_base = OTHER; note = thymine
  modified_base 2413: mod_base = OTHER; note = thymine
  modified_base 2418: mod_base = OTHER; note = thymine
  modified_base 2428: mod_base = OTHER; note = thymine
  modified_base 2432: mod_base = OTHER; note = thymine
  modified_base 2436..2437: mod_base = OTHER; note = thymine
  modified_base 2444: mod_base = OTHER; note = thymine
  modified_base 2446: mod_base = OTHER; note = thymine
  modified_base 2448: mod_base = OTHER; note = thymine
  modified_base 2451: mod_base = OTHER; note = thymine
  modified_base 2453: mod_base = OTHER; note = thymine
  modified_base 2458..2460: mod_base = OTHER; note = thymine
  modified_base 2462: mod_base = OTHER; note = thymine
  modified_base 2464: mod_base = OTHER; note = thymine
  modified_base 2467..2469: mod_base = OTHER; note = thymine
  modified_base 2472: mod_base = OTHER; note = thymine
  modified_base 2474..2475: mod_base = OTHER; note = thymine
  modified_base 2479: mod_base = OTHER; note = thymine
  modified_base 2490: mod_base = OTHER; note = thymine
  modified_base 2492..2493: mod_base = OTHER; note = thymine
  modified_base 2501: mod_base = OTHER; note = thymine
  modified_base 2507: mod_base = OTHER; note = thymine
  modified_base 2514: mod_base = OTHER; note = thymine
  modified_base 2517: mod_base = OTHER; note = thymine
  modified_base 2522: mod_base = OTHER; note = thymine
  modified_base 2527: mod_base = OTHER; note = thymine
  modified_base 2530..2531: mod_base = OTHER; note = thymine
  modified_base 2533: mod_base = OTHER; note = thymine
  modified_base 2538..2539: mod_base = OTHER; note = thymine
  modified_base 2542: mod_base = OTHER; note = thymine
  modified_base 2547..2550: mod_base = OTHER; note = thymine
  modified_base 2553: mod_base = OTHER; note = thymine
  modified_base 2560..2563: mod_base = OTHER; note = thymine
  modified_base 2567: mod_base = OTHER; note = thymine
  modified_base 2569: mod_base = OTHER; note = thymine
  modified_base 2571..2572: mod_base = OTHER; note = thymine
  modified_base 2575: mod_base = OTHER; note = thymine
  modified_base 2577: mod_base = OTHER; note = thymine
  modified_base 2580..2581: mod_base = OTHER; note = thymine
  modified_base 2588..2590: mod_base = OTHER; note = thymine
  modified_base 2593: mod_base = OTHER; note = thymine
  modified_base 2601..2602: mod_base = OTHER; note = thymine
  modified_base 2605..2606: mod_base = OTHER; note = thymine
  modified_base 2608: mod_base = OTHER; note = thymine
  modified_base 2614..2615: mod_base = OTHER; note = thymine
  modified_base 2617: mod_base = OTHER; note = thymine
  modified_base 2619: mod_base = OTHER; note = thymine
  modified_base 2622..2623: mod_base = OTHER; note = thymine
  modified_base 2625: mod_base = OTHER; note = thymine
  modified_base 2628: mod_base = OTHER; note = thymine
  modified_base 2638: mod_base = OTHER; note = thymine
  modified_base 2642..2643: mod_base = OTHER; note = thymine
  modified_base 2647..2650: mod_base = OTHER; note = thymine
  modified_base 2653: mod_base = OTHER; note = thymine
  modified_base 2655: mod_base = OTHER; note = thymine
  modified_base 2657: mod_base = OTHER; note = thymine
  modified_base 2664: mod_base = OTHER; note = thymine
  modified_base 2669: mod_base = OTHER; note = thymine
  modified_base 2671: mod_base = OTHER; note = thymine
  modified_base 2679: mod_base = OTHER; note = thymine
  modified_base 2688: mod_base = OTHER; note = thymine
  modified_base 2690..2692: mod_base = OTHER; note = thymine
  modified_base 2697: mod_base = OTHER; note = thymine
  modified_base 2699..2700: mod_base = OTHER; note = thymine
  modified_base 2702..2703: mod_base = OTHER; note = thymine
  modified_base 2705..2706: mod_base = OTHER; note = thymine
  modified_base 2710: mod_base = OTHER; note = thymine
  modified_base 2714: mod_base = OTHER; note = thymine
  modified_base 2725: mod_base = OTHER; note = thymine
  modified_base 2727: mod_base = OTHER; note = thymine
  modified_base 2729: mod_base = OTHER; note = thymine
  modified_base 2731: mod_base = OTHER; note = thymine
  modified_base 2733: mod_base = OTHER; note = thymine
  modified_base 2735: mod_base = OTHER; note = thymine
  modified_base 2742: mod_base = OTHER; note = thymine
  modified_base 2744: mod_base = OTHER; note = thymine
  modified_base 2746: mod_base = OTHER; note = thymine
  modified_base 2749: mod_base = OTHER; note = thymine
  modified_base 2753..2754: mod_base = OTHER; note = thymine
  modified_base 2757..2758: mod_base = OTHER; note = thymine
  modified_base 2762: mod_base = OTHER; note = thymine
  modified_base 2764: mod_base = OTHER; note = thymine
  modified_base 2767: mod_base = OTHER; note = thymine
  modified_base 2773: mod_base = OTHER; note = thymine
  modified_base 2775..2777: mod_base = OTHER; note = thymine
  modified_base 2783: mod_base = OTHER; note = thymine
  modified_base 2785: mod_base = OTHER; note = thymine
  modified_base 2787: mod_base = OTHER; note = thymine
  modified_base 2791: mod_base = OTHER; note = thymine
  modified_base 2794: mod_base = OTHER; note = thymine
  modified_base 2802..2803: mod_base = OTHER; note = thymine
  modified_base 2809: mod_base = OTHER; note = thymine
  modified_base 2813..2814: mod_base = OTHER; note = thymine
  modified_base 2817: mod_base = OTHER; note = thymine
  modified_base 2824..2825: mod_base = OTHER; note = thymine
  modified_base 2827: mod_base = OTHER; note = thymine
  modified_base 2829: mod_base = OTHER; note = thymine
  modified_base 2831: mod_base = OTHER; note = thymine
  modified_base 2833: mod_base = OTHER; note = thymine
  modified_base 2836: mod_base = OTHER; note = thymine
  modified_base 2838: mod_base = OTHER; note = thymine
  modified_base 2840: mod_base = OTHER; note = thymine
  modified_base 2846..2847: mod_base = OTHER; note = thymine
  modified_base 2849: mod_base = OTHER; note = thymine
  modified_base 2851..2852: mod_base = OTHER; note = thymine
  modified_base 2857..2860: mod_base = OTHER; note = thymine
  modified_base 2865..2866: mod_base = OTHER; note = thymine
  modified_base 2871..2872: mod_base = OTHER; note = thymine
  modified_base 2874: mod_base = OTHER; note = thymine
  modified_base 2876: mod_base = OTHER; note = thymine
  modified_base 2895..2896: mod_base = OTHER; note = thymine
  modified_base 2899: mod_base = OTHER; note = thymine
  modified_base 2903: mod_base = OTHER; note = thymine
  modified_base 2911: mod_base = OTHER; note = thymine
  modified_base 2913: mod_base = OTHER; note = thymine
  modified_base 2915..2916: mod_base = OTHER; note = thymine
  modified_base 2923: mod_base = OTHER; note = thymine
  modified_base 2933: mod_base = OTHER; note = thymine
  modified_base 2935..2936: mod_base = OTHER; note = thymine
  modified_base 2940..2941: mod_base = OTHER; note = thymine
  modified_base 2946: mod_base = OTHER; note = thymine
  modified_base 2959..2960: mod_base = OTHER; note = thymine
  modified_base 2963: mod_base = OTHER; note = thymine
  modified_base 2965: mod_base = OTHER; note = thymine
  modified_base 2971: mod_base = OTHER; note = thymine
  modified_base 2973..2975: mod_base = OTHER; note = thymine
  modified_base 2980: mod_base = OTHER; note = thymine
  modified_base 2983: mod_base = OTHER; note = thymine
  modified_base 2988..2989: mod_base = OTHER; note = thymine
  modified_base 2991..2993: mod_base = OTHER; note = thymine
  modified_base 2996..2998: mod_base = OTHER; note = thymine
  modified_base 3005: mod_base = OTHER; note = thymine
  modified_base 3011..3012: mod_base = OTHER; note = thymine
  modified_base 3015: mod_base = OTHER; note = thymine
  modified_base 3028..3030: mod_base = OTHER; note = thymine
  modified_base 3038..3039: mod_base = OTHER; note = thymine
  modified_base 3041: mod_base = OTHER; note = thymine
  modified_base 3043: mod_base = OTHER; note = thymine
  modified_base 3046: mod_base = OTHER; note = thymine
  modified_base 3048: mod_base = OTHER; note = thymine
  modified_base 3050..3051: mod_base = OTHER; note = thymine
  modified_base 3057..3060: mod_base = OTHER; note = thymine
  modified_base 3063: mod_base = OTHER; note = thymine
  modified_base 3067: mod_base = OTHER; note = thymine
  modified_base 3079..3083: mod_base = OTHER; note = thymine
  modified_base 3085: mod_base = OTHER; note = thymine
  modified_base 3091: mod_base = OTHER; note = thymine
  modified_base 3096: mod_base = OTHER; note = thymine
  modified_base 3099: mod_base = OTHER; note = thymine
  modified_base 3102: mod_base = OTHER; note = thymine
  modified_base 3106: mod_base = OTHER; note = thymine
  modified_base 3112: mod_base = OTHER; note = thymine
  modified_base 3114: mod_base = OTHER; note = thymine
  modified_base 3118: mod_base = OTHER; note = thymine
  modified_base 3125: mod_base = OTHER; note = thymine
  modified_base 3127: mod_base = OTHER; note = thymine
  modified_base 3132: mod_base = OTHER; note = thymine
  modified_base 3135: mod_base = OTHER; note = thymine
  modified_base 3140: mod_base = OTHER; note = thymine
  modified_base 3144: mod_base = OTHER; note = thymine
  modified_base 3149: mod_base = OTHER; note = thymine
  modified_base 3153: mod_base = OTHER; note = thymine
  modified_base 3164..3165: mod_base = OTHER; note = thymine
  modified_base 3167..3168: mod_base = OTHER; note = thymine
  modified_base 3172: mod_base = OTHER; note = thymine
  modified_base 3175..3176: mod_base = OTHER; note = thymine
  modified_base 3183: mod_base = OTHER; note = thymine
  modified_base 3185: mod_base = OTHER; note = thymine
  modified_base 3189: mod_base = OTHER; note = thymine
  modified_base 3191: mod_base = OTHER; note = thymine
  modified_base 3194: mod_base = OTHER; note = thymine
  modified_base 3196: mod_base = OTHER; note = thymine
  modified_base 3198..3199: mod_base = OTHER; note = thymine
  modified_base 3201: mod_base = OTHER; note = thymine
  modified_base 3208..3209: mod_base = OTHER; note = thymine
  modified_base 3213: mod_base = OTHER; note = thymine
  modified_base 3220: mod_base = OTHER; note = thymine
  modified_base 3222: mod_base = OTHER; note = thymine
  modified_base 3235..3236: mod_base = OTHER; note = thymine
  modified_base 3238: mod_base = OTHER; note = thymine
  modified_base 3240: mod_base = OTHER; note = thymine
  modified_base 3244: mod_base = OTHER; note = thymine
  modified_base 3252: mod_base = OTHER; note = thymine
  modified_base 3255: mod_base = OTHER; note = thymine
  modified_base 3259..3260: mod_base = OTHER; note = thymine
  modified_base 3264: mod_base = OTHER; note = thymine
  modified_base 3270: mod_base = OTHER; note = thymine
  modified_base 3273: mod_base = OTHER; note = thymine
  modified_base 3276: mod_base = OTHER; note = thymine
  modified_base 3285: mod_base = OTHER; note = thymine
  modified_base 3287..3288: mod_base = OTHER; note = thymine
  modified_base 3298: mod_base = OTHER; note = thymine
  modified_base 3301: mod_base = OTHER; note = thymine
  modified_base 3306: mod_base = OTHER; note = thymine
  modified_base 3316..3317: mod_base = OTHER; note = thymine
  modified_base 3323: mod_base = OTHER; note = thymine
  modified_base 3329: mod_base = OTHER; note = thymine
  modified_base 3331: mod_base = OTHER; note = thymine
  modified_base 3333..3334: mod_base = OTHER; note = thymine
  modified_base 3338..3340: mod_base = OTHER; note = thymine
  modified_base 3344: mod_base = OTHER; note = thymine
  modified_base 3351: mod_base = OTHER; note = thymine
  modified_base 3356..3357: mod_base = OTHER; note = thymine
  modified_base 3359: mod_base = OTHER; note = thymine
  modified_base 3366..3367: mod_base = OTHER; note = thymine
  modified_base 3370..3371: mod_base = OTHER; note = thymine
  modified_base 3377..3378: mod_base = OTHER; note = thymine
  modified_base 3387..3388: mod_base = OTHER; note = thymine
  modified_base 3390: mod_base = OTHER; note = thymine
  modified_base 3396: mod_base = OTHER; note = thymine
  modified_base 3401..3403: mod_base = OTHER; note = thymine
  modified_base 3409: mod_base = OTHER; note = thymine
  modified_base 3411..3412: mod_base = OTHER; note = thymine
  modified_base 3415..3416: mod_base = OTHER; note = thymine
  modified_base 3419: mod_base = OTHER; note = thymine
  modified_base 3422..3423: mod_base = OTHER; note = thymine
  modified_base 3429..3430: mod_base = OTHER; note = thymine
  modified_base 3435..3437: mod_base = OTHER; note = thymine
  modified_base 3439..3440: mod_base = OTHER; note = thymine
  modified_base 3442: mod_base = OTHER; note = thymine
  modified_base 3444: mod_base = OTHER; note = thymine
  modified_base 3446: mod_base = OTHER; note = thymine
  modified_base 3448: mod_base = OTHER; note = thymine
  modified_base 3453..3454: mod_base = OTHER; note = thymine
  modified_base 3456..3457: mod_base = OTHER; note = thymine
  modified_base 3461..3462: mod_base = OTHER; note = thymine
  modified_base 3464: mod_base = OTHER; note = thymine
  modified_base 3467: mod_base = OTHER; note = thymine
  modified_base 3469: mod_base = OTHER; note = thymine
  modified_base 3471: mod_base = OTHER; note = thymine
  modified_base 3491: mod_base = OTHER; note = thymine
  modified_base 3494: mod_base = OTHER; note = thymine
  modified_base 3499: mod_base = OTHER; note = thymine
  modified_base 3502: mod_base = OTHER; note = thymine
  modified_base 3505..3506: mod_base = OTHER; note = thymine
  modified_base 3509: mod_base = OTHER; note = thymine
  modified_base 3515..3517: mod_base = OTHER; note = thymine
  modified_base 3522: mod_base = OTHER; note = thymine
  modified_base 3525: mod_base = OTHER; note = thymine
  modified_base 3528: mod_base = OTHER; note = thymine
  modified_base 3530: mod_base = OTHER; note = thymine
  modified_base 3543: mod_base = OTHER; note = thymine
  modified_base 3549..3550: mod_base = OTHER; note = thymine
  modified_base 3552..3553: mod_base = OTHER; note = thymine
  modified_base 3562..3565: mod_base = OTHER; note = thymine
  modified_base 3568: mod_base = OTHER; note = thymine
  modified_base 3570: mod_base = OTHER; note = thymine
  modified_base 3577..3578: mod_base = OTHER; note = thymine
  modified_base 3584..3585: mod_base = OTHER; note = thymine
  modified_base 3590..3591: mod_base = OTHER; note = thymine
  modified_base 3594..3597: mod_base = OTHER; note = thymine
  modified_base 3599: mod_base = OTHER; note = thymine
  modified_base 3603..3604: mod_base = OTHER; note = thymine
  modified_base 3611: mod_base = OTHER; note = thymine
  modified_base 3613: mod_base = OTHER; note = thymine
  modified_base 3615: mod_base = OTHER; note = thymine
  modified_base 3617..3619: mod_base = OTHER; note = thymine
  modified_base 3622..3623: mod_base = OTHER; note = thymine
  modified_base 3628: mod_base = OTHER; note = thymine
  modified_base 3638..3639: mod_base = OTHER; note = thymine
  modified_base 3641: mod_base = OTHER; note = thymine
  modified_base 3648: mod_base = OTHER; note = thymine
  modified_base 3650..3651: mod_base = OTHER; note = thymine
  modified_base 3656: mod_base = OTHER; note = thymine
  modified_base 3661: mod_base = OTHER; note = thymine
  modified_base 3664: mod_base = OTHER; note = thymine
  modified_base 3667: mod_base = OTHER; note = thymine
  modified_base 3672..3673: mod_base = OTHER; note = thymine
  modified_base 3676: mod_base = OTHER; note = thymine
  modified_base 3678..3679: mod_base = OTHER; note = thymine
  modified_base 3684: mod_base = OTHER; note = thymine
  modified_base 3686: mod_base = OTHER; note = thymine
  modified_base 3693: mod_base = OTHER; note = thymine
  modified_base 3698..3699: mod_base = OTHER; note = thymine
  modified_base 3701: mod_base = OTHER; note = thymine
  modified_base 3703: mod_base = OTHER; note = thymine
  modified_base 3705..3707: mod_base = OTHER; note = thymine
  modified_base 3717: mod_base = OTHER; note = thymine
  modified_base 3720: mod_base = OTHER; note = thymine
  modified_base 3723..3724: mod_base = OTHER; note = thymine
  modified_base 3726: mod_base = OTHER; note = thymine
  modified_base 3730..3731: mod_base = OTHER; note = thymine
  modified_base 3734: mod_base = OTHER; note = thymine
  modified_base 3740..3742: mod_base = OTHER; note = thymine
  modified_base 3747..3748: mod_base = OTHER; note = thymine
  modified_base 3752: mod_base = OTHER; note = thymine
  modified_base 3754: mod_base = OTHER; note = thymine
  modified_base 3756..3758: mod_base = OTHER; note = thymine
  modified_base 3763: mod_base = OTHER; note = thymine
  modified_base 3766: mod_base = OTHER; note = thymine
  modified_base 3769..3770: mod_base = OTHER; note = thymine
  modified_base 3772: mod_base = OTHER; note = thymine
  modified_base 3777: mod_base = OTHER; note = thymine
  modified_base 3779: mod_base = OTHER; note = thymine
  modified_base 3781: mod_base = OTHER; note = thymine
  modified_base 3789: mod_base = OTHER; note = thymine
  modified_base 3792: mod_base = OTHER; note = thymine
  modified_base 3794: mod_base = OTHER; note = thymine
  modified_base 3798: mod_base = OTHER; note = thymine
  modified_base 3806: mod_base = OTHER; note = thymine
  modified_base 3810..3811: mod_base = OTHER; note = thymine
  modified_base 3815: mod_base = OTHER; note = thymine
  modified_base 3821: mod_base = OTHER; note = thymine
  modified_base 3825..3827: mod_base = OTHER; note = thymine
  modified_base 3829: mod_base = OTHER; note = thymine
  modified_base 3834: mod_base = OTHER; note = thymine
  modified_base 3838..3841: mod_base = OTHER; note = thymine
  modified_base 3843: mod_base = OTHER; note = thymine
  modified_base 3845: mod_base = OTHER; note = thymine
  modified_base 3849..3850: mod_base = OTHER; note = thymine
  modified_base 3852: mod_base = OTHER; note = thymine
  modified_base 3859: mod_base = OTHER; note = thymine
  modified_base 3863..3864: mod_base = OTHER; note = thymine
  modified_base 3870..3871: mod_base = OTHER; note = thymine
  modified_base 3874..3876: mod_base = OTHER; note = thymine
  modified_base 3880: mod_base = OTHER; note = thymine
  modified_base 3883: mod_base = OTHER; note = thymine
  modified_base 3886: mod_base = OTHER; note = thymine
  modified_base 3897: mod_base = OTHER; note = thymine
  modified_base 3900: mod_base = OTHER; note = thymine
  modified_base 3903..3905: mod_base = OTHER; note = thymine
  modified_base 3908: mod_base = OTHER; note = thymine
  modified_base 3910: mod_base = OTHER; note = thymine
  modified_base 3917..3918: mod_base = OTHER; note = thymine
  modified_base 3922: mod_base = OTHER; note = thymine
  modified_base 3935: mod_base = OTHER; note = thymine
  modified_base 3937: mod_base = OTHER; note = thymine
  modified_base 3941: mod_base = OTHER; note = thymine
  modified_base 3943: mod_base = OTHER; note = thymine
  modified_base 3946..3947: mod_base = OTHER; note = thymine
  modified_base 3949: mod_base = OTHER; note = thymine
  modified_base 3951: mod_base = OTHER; note = thymine
  modified_base 3954: mod_base = OTHER; note = thymine
  modified_base 3957: mod_base = OTHER; note = thymine
  modified_base 3961: mod_base = OTHER; note = thymine
  modified_base 3968..3970: mod_base = OTHER; note = thymine
  modified_base 3972: mod_base = OTHER; note = thymine
  modified_base 3975: mod_base = OTHER; note = thymine
  modified_base 3978: mod_base = OTHER; note = thymine
  modified_base 3981: mod_base = OTHER; note = thymine
  modified_base 3983: mod_base = OTHER; note = thymine
  modified_base 3986..3987: mod_base = OTHER; note = thymine
  modified_base 3991..3992: mod_base = OTHER; note = thymine
  modified_base 3999: mod_base = OTHER; note = thymine
  modified_base 4002..4003: mod_base = OTHER; note = thymine
  modified_base 4010..4011: mod_base = OTHER; note = thymine
  modified_base 4019..4020: mod_base = OTHER; note = thymine
  modified_base 4023: mod_base = OTHER; note = thymine
  modified_base 4025..4026: mod_base = OTHER; note = thymine
  modified_base 4028..4029: mod_base = OTHER; note = thymine
  modified_base 4032: mod_base = OTHER; note = thymine
  modified_base 4037: mod_base = OTHER; note = thymine
  modified_base 4039..4040: mod_base = OTHER; note = thymine
  modified_base 4042..4043: mod_base = OTHER; note = thymine
  modified_base 4045: mod_base = OTHER; note = thymine
  modified_base 4050..4055: mod_base = OTHER; note = thymine
  modified_base 4058: mod_base = OTHER; note = thymine
  modified_base 4078: mod_base = OTHER; note = thymine
  modified_base 4080: mod_base = OTHER; note = thymine
  modified_base 4083: mod_base = OTHER; note = thymine
  modified_base 4088: mod_base = OTHER; note = thymine
  modified_base 4091..4092: mod_base = OTHER; note = thymine
  modified_base 4101: mod_base = OTHER; note = thymine
  modified_base 4105: mod_base = OTHER; note = thymine
  modified_base 4109..4111: mod_base = OTHER; note = thymine
  modified_base 4115: mod_base = OTHER; note = thymine
  modified_base 4121: mod_base = OTHER; note = thymine
  modified_base 4125: mod_base = OTHER; note = thymine
  modified_base 4134: mod_base = OTHER; note = thymine
  modified_base 4139: mod_base = OTHER; note = thymine
  modified_base 4143: mod_base = OTHER; note = thymine
  modified_base 4145..4147: mod_base = OTHER; note = thymine
  modified_base 4149: mod_base = OTHER; note = thymine
  modified_base 4155: mod_base = OTHER; note = thymine
  modified_base 4162: mod_base = OTHER; note = thymine
  modified_base 4170: mod_base = OTHER; note = thymine
  modified_base 4172: mod_base = OTHER; note = thymine
  modified_base 4176: mod_base = OTHER; note = thymine
  modified_base 4182: mod_base = OTHER; note = thymine
  modified_base 4185..4186: mod_base = OTHER; note = thymine
  modified_base 4191: mod_base = OTHER; note = thymine
  modified_base 4194: mod_base = OTHER; note = thymine
  modified_base 4198..4199: mod_base = OTHER; note = thymine
  modified_base 4206: mod_base = OTHER; note = thymine
  modified_base 4208: mod_base = OTHER; note = thymine
  modified_base 4211..4213: mod_base = OTHER; note = thymine
  modified_base 4216: mod_base = OTHER; note = thymine
  modified_base 4218: mod_base = OTHER; note = thymine
  modified_base 4220: mod_base = OTHER; note = thymine
  modified_base 4222: mod_base = OTHER; note = thymine
  modified_base 4229: mod_base = OTHER; note = thymine
  modified_base 4247: mod_base = OTHER; note = thymine
  modified_base 4251: mod_base = OTHER; note = thymine
  modified_base 4260: mod_base = OTHER; note = thymine
  modified_base 4262: mod_base = OTHER; note = thymine
  modified_base 4266: mod_base = OTHER; note = thymine
  modified_base 4269: mod_base = OTHER; note = thymine
  modified_base 4277: mod_base = OTHER; note = thymine
  modified_base 4281..4286: mod_base = OTHER; note = thymine
  modified_base 4289..4291: mod_base = OTHER; note = thymine
  modified_base 4296..4297: mod_base = OTHER; note = thymine
  modified_base 4300: mod_base = OTHER; note = thymine
  modified_base 4303: mod_base = OTHER; note = thymine
  modified_base 4305: mod_base = OTHER; note = thymine
  modified_base 4310..4313: mod_base = OTHER; note = thymine
  modified_base 4315: mod_base = OTHER; note = thymine
  modified_base 4320: mod_base = OTHER; note = thymine
  modified_base 4324: mod_base = OTHER; note = thymine
  modified_base 4326..4327: mod_base = OTHER; note = thymine
  modified_base 4333: mod_base = OTHER; note = thymine
  modified_base 4335..4336: mod_base = OTHER; note = thymine
  modified_base 4340: mod_base = OTHER; note = thymine
  modified_base 4343: mod_base = OTHER; note = thymine
  modified_base 4345: mod_base = OTHER; note = thymine
  modified_base 4348..4350: mod_base = OTHER; note = thymine
  modified_base 4352: mod_base = OTHER; note = thymine
  modified_base 4356: mod_base = OTHER; note = thymine
  modified_base 4359..4361: mod_base = OTHER; note = thymine
  modified_base 4363: mod_base = OTHER; note = thymine
  modified_base 4366: mod_base = OTHER; note = thymine
  modified_base 4371: mod_base = OTHER; note = thymine
  modified_base 4381..4382: mod_base = OTHER; note = thymine
  modified_base 4385..4386: mod_base = OTHER; note = thymine
  modified_base 4389: mod_base = OTHER; note = thymine
  modified_base 4392: mod_base = OTHER; note = thymine
  modified_base 4394: mod_base = OTHER; note = thymine
  modified_base 4396..4397: mod_base = OTHER; note = thymine
  modified_base 4402: mod_base = OTHER; note = thymine
  modified_base 4404: mod_base = OTHER; note = thymine
  modified_base 4407: mod_base = OTHER; note = thymine
  modified_base 4411: mod_base = OTHER; note = thymine
  modified_base 4415: mod_base = OTHER; note = thymine
  modified_base 4418: mod_base = OTHER; note = thymine
  modified_base 4421..4422: mod_base = OTHER; note = thymine
  modified_base 4424..4425: mod_base = OTHER; note = thymine
  modified_base 4427..4428: mod_base = OTHER; note = thymine
  modified_base 4431..4432: mod_base = OTHER; note = thymine
  modified_base 4435..4436: mod_base = OTHER; note = thymine
  modified_base 4438: mod_base = OTHER; note = thymine
  modified_base 4441..4442: mod_base = OTHER; note = thymine
  modified_base 4444..4445: mod_base = OTHER; note = thymine
  modified_base 4453: mod_base = OTHER; note = thymine
  modified_base 4456: mod_base = OTHER; note = thymine
  modified_base 4466: mod_base = OTHER; note = thymine
  modified_base 4468..4469: mod_base = OTHER; note = thymine
  modified_base 4473..4478: mod_base = OTHER; note = thymine
  modified_base 4481: mod_base = OTHER; note = thymine
  modified_base 4483..4484: mod_base = OTHER; note = thymine
  modified_base 4488: mod_base = OTHER; note = thymine
  modified_base 4491: mod_base = OTHER; note = thymine
  modified_base 4495: mod_base = OTHER; note = thymine
  modified_base 4500: mod_base = OTHER; note = thymine
  modified_base 4506: mod_base = OTHER; note = thymine
  modified_base 4512..4513: mod_base = OTHER; note = thymine
  modified_base 4515: mod_base = OTHER; note = thymine
  modified_base 4519: mod_base = OTHER; note = thymine
  modified_base 4523..4526: mod_base = OTHER; note = thymine
  modified_base 4528..4531: mod_base = OTHER; note = thymine
  modified_base 4537: mod_base = OTHER; note = thymine
  modified_base 4539: mod_base = OTHER; note = thymine
  modified_base 4546: mod_base = OTHER; note = thymine
  modified_base 4549: mod_base = OTHER; note = thymine
  modified_base 4559: mod_base = OTHER; note = thymine
  modified_base 4561..4562: mod_base = OTHER; note = thymine
  modified_base 4571..4573: mod_base = OTHER; note = thymine
  modified_base 4575: mod_base = OTHER; note = thymine
  modified_base 4578..4579: mod_base = OTHER; note = thymine
  modified_base 4581: mod_base = OTHER; note = thymine
  modified_base 4584: mod_base = OTHER; note = thymine
  modified_base 4597: mod_base = OTHER; note = thymine
  modified_base 4599..4601: mod_base = OTHER; note = thymine
  modified_base 4607..4608: mod_base = OTHER; note = thymine
  modified_base 4611: mod_base = OTHER; note = thymine
  modified_base 4617..4618: mod_base = OTHER; note = thymine
  modified_base 4624..4625: mod_base = OTHER; note = thymine
  modified_base 4633..4636: mod_base = OTHER; note = thymine
  modified_base 4638..4639: mod_base = OTHER; note = thymine
  modified_base 4642..4643: mod_base = OTHER; note = thymine
  modified_base 4650: mod_base = OTHER; note = thymine
  modified_base 4658: mod_base = OTHER; note = thymine
  modified_base 4660: mod_base = OTHER; note = thymine
  modified_base 4663: mod_base = OTHER; note = thymine
  modified_base 4666: mod_base = OTHER; note = thymine
  modified_base 4669: mod_base = OTHER; note = thymine
  modified_base 4671..4672: mod_base = OTHER; note = thymine
  modified_base 4678: mod_base = OTHER; note = thymine
  modified_base 4680: mod_base = OTHER; note = thymine
  modified_base 4686..4687: mod_base = OTHER; note = thymine
  modified_base 4690..4693: mod_base = OTHER; note = thymine
  modified_base 4697..4698: mod_base = OTHER; note = thymine
  modified_base 4703: mod_base = OTHER; note = thymine
  modified_base 4705: mod_base = OTHER; note = thymine
  modified_base 4709..4711: mod_base = OTHER; note = thymine
  modified_base 4715: mod_base = OTHER; note = thymine
  modified_base 4717..4718: mod_base = OTHER; note = thymine
  modified_base 4721: mod_base = OTHER; note = thymine
  modified_base 4723: mod_base = OTHER; note = thymine
  modified_base 4725..4726: mod_base = OTHER; note = thymine
  modified_base 4738: mod_base = OTHER; note = thymine
  modified_base 4753: mod_base = OTHER; note = thymine
  modified_base 4755: mod_base = OTHER; note = thymine
  modified_base 4757..4759: mod_base = OTHER; note = thymine
  modified_base 4766: mod_base = OTHER; note = thymine
  modified_base 4769: mod_base = OTHER; note = thymine
  modified_base 4774..4776: mod_base = OTHER; note = thymine
  modified_base 4780: mod_base = OTHER; note = thymine
  modified_base 4782: mod_base = OTHER; note = thymine
  modified_base 4784: mod_base = OTHER; note = thymine
  modified_base 4792: mod_base = OTHER; note = thymine
  modified_base 4794..4795: mod_base = OTHER; note = thymine
  modified_base 4800: mod_base = OTHER; note = thymine
  modified_base 4802..4804: mod_base = OTHER; note = thymine
  source 1..7114: mol_type = other RNA

SEQ ID NO: 5 - RNA, 3377 nt, unidentified
  modified_base 13: mod_base = OTHER; note = thymine
  modified_base 16: mod_base = OTHER; note = thymine
  modified_base 21..25: mod_base = OTHER; note = thymine
  modified_base 29..30: mod_base = OTHER; note = thymine
  modified_base 35..37: mod_base = OTHER; note = thymine
  modified_base 40: mod_base = OTHER; note = thymine
  modified_base 42: mod_base = OTHER; note = thymine
  modified_base 46: mod_base = OTHER; note = thymine
  modified_base 51: mod_base = OTHER; note = thymine
  modified_base 55: mod_base = OTHER; note = thymine
  modified_base 57: mod_base = OTHER; note = thymine
  modified_base 65: mod_base = OTHER; note = thymine
  modified_base 67: mod_base = OTHER; note = thymine
  modified_base 70: mod_base = OTHER; note = thymine
  modified_base 80: mod_base = OTHER; note = thymine
  modified_base 84: mod_base = OTHER; note = thymine
  modified_base 89..90: mod_base = OTHER; note = thymine
  modified_base 92: mod_base = OTHER; note = thymine
  modified_base 98: mod_base = OTHER; note = thymine
  modified_base 100..102: mod_base = OTHER; note = thymine
  modified_base 108: mod_base = OTHER; note = thymine
  modified_base 110: mod_base = OTHER; note = thymine
  modified_base 114: mod_base = OTHER; note = thymine
  modified_base 119: mod_base = OTHER; note = thymine
  modified_base 122: mod_base = OTHER; note = thymine
  modified_base 126: mod_base = OTHER; note = thymine
  modified_base 131: mod_base = OTHER; note = thymine
  modified_base 134: mod_base = OTHER; note = thymine
  modified_base 140..141: mod_base = OTHER; note = thymine
  modified_base 143: mod_base = OTHER; note = thymine
  modified_base 146..147: mod_base = OTHER; note = thymine
  modified_base 149..150: mod_base = OTHER; note = thymine
  modified_base 152: mod_base = OTHER; note = thymine
  modified_base 155..156: mod_base = OTHER; note = thymine
  modified_base 158: mod_base = OTHER; note = thymine
  modified_base 161: mod_base = OTHER; note = thymine
  modified_base 164: mod_base = OTHER; note = thymine
  modified_base 171: mod_base = OTHER; note = thymine
  modified_base 173: mod_base = OTHER; note = thymine
  modified_base 180: mod_base = OTHER; note = thymine
  modified_base 182: mod_base = OTHER; note = thymine
  modified_base 184: mod_base = OTHER; note = thymine
  modified_base 189..192: mod_base = OTHER; note = thymine
  modified_base 199: mod_base = OTHER; note = thymine
  modified_base 201: mod_base = OTHER; note = thymine
  modified_base 206: mod_base = OTHER; note = thymine
  modified_base 208..209: mod_base = OTHER; note = thymine
  modified_base 214..215: mod_base = OTHER; note = thymine
  modified_base 218: mod_base = OTHER; note = thymine
  modified_base 222..224: mod_base = OTHER; note = thymine
  modified_base 227: mod_base = OTHER; note = thymine
  modified_base 230..231: mod_base = OTHER; note = thymine
  modified_base 233: mod_base = OTHER; note = thymine
  modified_base 237: mod_base = OTHER; note = thymine
  modified_base 244: mod_base = OTHER; note = thymine
  modified_base 247: mod_base = OTHER; note = thymine
  modified_base 249: mod_base = OTHER; note = thymine
  modified_base 254: mod_base = OTHER; note = thymine
  modified_base 256: mod_base = OTHER; note = thymine
  modified_base 261: mod_base = OTHER; note = thymine
  modified_base 263..264: mod_base = OTHER; note = thymine
  modified_base 278..280: mod_base = OTHER; note = thymine
  modified_base 290..292: mod_base = OTHER; note = thymine
  modified_base 298..300: mod_base = OTHER; note = thymine
  modified_base 304: mod_base = OTHER; note = thymine
  modified_base 308: mod_base = OTHER; note = thymine
  modified_base 313..315: mod_base = OTHER; note = thymine
  modified_base 318: mod_base = OTHER; note = thymine
  modified_base 320: mod_base = OTHER; note = thymine
  modified_base 322: mod_base = OTHER; note = thymine
  modified_base 327: mod_base = OTHER; note = thymine
  modified_base 329: mod_base = OTHER; note = thymine
  modified_base 335: mod_base = OTHER; note = thymine
  modified_base 341: mod_base = OTHER; note = thymine
  modified_base 352: mod_base = OTHER; note = thymine
  modified_base 354..355: mod_base = OTHER; note = thymine
  modified_base 367: mod_base = OTHER; note = thymine
  modified_base 375..376: mod_base = OTHER; note = thymine
  modified_base 380: mod_base = OTHER; note = thymine
  modified_base 384: mod_base = OTHER; note = thymine
  modified_base 386: mod_base = OTHER; note = thymine
  modified_base 393..394: mod_base = OTHER; note = thymine
  modified_base 396: mod_base = OTHER; note = thymine
  modified_base 401: mod_base = OTHER; note = thymine
  modified_base 406..407: mod_base = OTHER; note = thymine
  modified_base 412..413: mod_base = OTHER; note = thymine
  modified_base 420: mod_base = OTHER; note = thymine
  modified_base 423..424: mod_base = OTHER; note = thymine
  modified_base 428: mod_base = OTHER; note = thymine
  modified_base 431: mod_base = OTHER; note = thymine
  modified_base 435: mod_base = OTHER; note = thymine
  modified_base 442..445: mod_base = OTHER; note = thymine
  modified_base 450: mod_base = OTHER; note = thymine
  modified_base 452: mod_base = OTHER; note = thymine
  modified_base 457: mod_base = OTHER; note = thymine
  modified_base 459: mod_base = OTHER; note = thymine
  modified_base 463..464: mod_base = OTHER; note = thymine
  modified_base 478..480: mod_base = OTHER; note = thymine
  modified_base 491: mod_base = OTHER; note = thymine
  modified_base 494: mod_base = OTHER; note = thymine
  modified_base 498: mod_base = OTHER; note = thymine
  modified_base 500: mod_base = OTHER; note = thymine
  modified_base 503..505: mod_base = OTHER; note = thymine
  modified_base 512: mod_base = OTHER; note = thymine
  modified_base 518: mod_base = OTHER; note = thymine
  modified_base 522: mod_base = OTHER; note = thymine
  modified_base 524: mod_base = OTHER; note = thymine
  modified_base 535: mod_base = OTHER; note = thymine
  modified_base 541: mod_base = OTHER; note = thymine
  modified_base 543: mod_base = OTHER; note = thymine
  modified_base 551: mod_base = OTHER; note = thymine
  modified_base 553: mod_base = OTHER; note = thymine
  modified_base 556: mod_base = OTHER; note = thymine
  modified_base 562..563: mod_base = OTHER; note = thymine
  modified_base 570: mod_base = OTHER; note = thymine
  modified_base 575: mod_base = OTHER; note = thymine
  modified_base 581: mod_base = OTHER; note = thymine
  modified_base 586: mod_base = OTHER; note = thymine
  modified_base 592..593: mod_base = OTHER; note = thymine
  modified_base 598..599: mod_base = OTHER; note = thymine
  modified_base 601: mod_base = OTHER; note = thymine
  modified_base 606: mod_base = OTHER; note = thymine
  modified_base 615: mod_base = OTHER; note = thymine
  modified_base 618: mod_base = OTHER; note = thymine
  modified_base 628: mod_base = OTHER; note = thymine
  modified_base 630: mod_base = OTHER; note = thymine
  modified_base 637..638: mod_base = OTHER; note = thymine
  modified_base 645: mod_base = OTHER; note = thymine
  modified_base 650: mod_base = OTHER; note = thymine
  modified_base 653: mod_base = OTHER; note = thymine
  modified_base 657: mod_base = OTHER; note = thymine
  modified_base 659: mod_base = OTHER; note = thymine
  modified_base 661..663: mod_base = OTHER; note = thymine
  modified_base 669: mod_base = OTHER; note = thymine
  modified_base 673..674: mod_base = OTHER; note = thymine
  modified_base 685: mod_base = OTHER; note = thymine
  modified_base 689: mod_base = OTHER; note = thymine
  modified_base 696: mod_base = OTHER; note = thymine
  modified_base 700: mod_base = OTHER; note = thymine
  modified_base 706: mod_base = OTHER; note = thymine
  modified_base 715: mod_base = OTHER; note = thymine
  modified_base 720: mod_base = OTHER; note = thymine
  modified_base 733: mod_base = OTHER; note = thymine
  modified_base 736: mod_base = OTHER; note = thymine
  modified_base 738: mod_base = OTHER; note = thymine
  modified_base 750..751: mod_base = OTHER; note = thymine
  modified_base 757: mod_base = OTHER; note = thymine
  modified_base 761: mod_base = OTHER; note = thymine
  modified_base 764..765: mod_base = OTHER; note = thymine
  modified_base 767: mod_base = OTHER; note = thymine
  modified_base 774: mod_base = OTHER; note = thymine
  modified_base 781: mod_base = OTHER; note = thymine
  modified_base 793: mod_base = OTHER; note = thymine
  modified_base 799: mod_base = OTHER; note = thymine
  modified_base 801: mod_base = OTHER; note = thymine
  modified_base 807: mod_base = OTHER; note = thymine
  modified_base 810..813: mod_base = OTHER; note = thymine
  modified_base 818: mod_base = OTHER; note = thymine
  modified_base 820: mod_base = OTHER; note = thymine
  modified_base 827..828: mod_base = OTHER; note = thymine
  modified_base 830: mod_base = OTHER; note = thymine
  modified_base 833..835: mod_base = OTHER; note = thymine
  modified_base 849: mod_base = OTHER; note = thymine
  modified_base 853..854: mod_base = OTHER; note = thymine
  modified_base 857: mod_base = OTHER; note = thymine
  modified_base 861: mod_base = OTHER; note = thymine
  modified_base 869: mod_base = OTHER; note = thymine
  modified_base 880..881: mod_base = OTHER; note = thymine
  modified_base 886: mod_base = OTHER; note = thymine
  modified_base 891..893: mod_base = OTHER; note = thymine
  modified_base 895: mod_base = OTHER; note = thymine
  modified_base 901: mod_base = OTHER; note = thymine
  modified_base 903..904: mod_base = OTHER; note = thymine
  modified_base 911: mod_base = OTHER; note = thymine
  modified_base 918: mod_base = OTHER; note = thymine
  modified_base 924: mod_base = OTHER; note = thymine
  modified_base 927..928: mod_base = OTHER; note = thymine
  modified_base 930..931: mod_base = OTHER; note = thymine
  modified_base 935: mod_base = OTHER; note = thymine
  modified_base 943: mod_base = OTHER; note = thymine
  modified_base 947: mod_base = OTHER; note = thymine
  modified_base 950..951: mod_base = OTHER; note = thymine
  modified_base 953..954: mod_base = OTHER; note = thymine
  modified_base 957: mod_base = OTHER; note = thymine
  modified_base 965..966: mod_base = OTHER; note = thymine
  modified_base 970: mod_base = OTHER; note = thymine
  modified_base 972..974: mod_base = OTHER; note = thymine
  modified_base 989..990: mod_base = OTHER; note = thymine
  modified_base 997: mod_base = OTHER; note = thymine
  modified_base 1002: mod_base = OTHER; note = thymine
  modified_base 1004: mod_base = OTHER; note = thymine
  modified_base 1008: mod_base = OTHER; note = thymine
  modified_base 1011: mod_base = OTHER; note = thymine
  modified_base 1014: mod_base = OTHER; note = thymine
  modified_base 1021..1022: mod_base = OTHER; note = thymine
  modified_base 1024: mod_base = OTHER; note = thymine
  modified_base 1026: mod_base = OTHER; note = thymine
  modified_base 1031: mod_base = OTHER; note = thymine
  modified_base 1034: mod_base = OTHER; note = thymine
  modified_base 1040: mod_base = OTHER; note = thymine
  modified_base 1043..1044: mod_base = OTHER; note = thymine
  modified_base 1048: mod_base = OTHER; note = thymine
  modified_base 1059: mod_base = OTHER; note = thymine
  modified_base 1062..1064: mod_base = OTHER; note = thymine
  modified_base 1068: mod_base = OTHER; note = thymine
  modified_base 1072..1073: mod_base = OTHER; note = thymine
  modified_base 1085: mod_base = OTHER; note = thymine
  modified_base 1090: mod_base = OTHER; note = thymine
  modified_base 1092: mod_base = OTHER; note = thymine
  modified_base 1096..1097: mod_base = OTHER; note = thymine
  modified_base 1102..1103: mod_base = OTHER; note = thymine
  modified_base 1105..1106: mod_base = OTHER; note = thymine
  modified_base 1118: mod_base = OTHER; note = thymine
  modified_base 1122..1123: mod_base = OTHER; note = thymine
  modified_base 1125..1127: mod_base = OTHER; note = thymine
  modified_base 1130..1132: mod_base = OTHER; note = thymine
  modified_base 1137: mod_base = OTHER; note = thymine
  modified_base 1142: mod_base = OTHER; note = thymine
  modified_base 1145: mod_base = OTHER; note = thymine
  modified_base 1148: mod_base = OTHER; note = thymine
  modified_base 1159..1160: mod_base = OTHER; note = thymine
  modified_base 1167: mod_base = OTHER; note = thymine
  modified_base 1169: mod_base = OTHER; note = thymine
  modified_base 1172: mod_base = OTHER; note = thymine
  modified_base 1178: mod_base = OTHER; note = thymine
  modified_base 1183: mod_base = OTHER; note = thymine
  modified_base 1185..1186: mod_base = OTHER; note = thymine
  modified_base 1191..1192: mod_base = OTHER; note = thymine
  modified_base 1195: mod_base = OTHER; note = thymine
  modified_base 1201..1204: mod_base = OTHER; note = thymine
  modified_base 1207: mod_base = OTHER; note = thymine
  modified_base 1211: mod_base = OTHER; note = thymine
  modified_base 1218: mod_base = OTHER; note = thymine
  modified_base 1236: mod_base = OTHER; note = thymine
  modified_base 1239: mod_base = OTHER; note = thymine
  modified_base 1241: mod_base = OTHER; note = thymine
  modified_base 1249: mod_base = OTHER; note = thymine
  modified_base 1252: mod_base = OTHER; note = thymine
  modified_base 1256..1257: mod_base = OTHER; note = thymine
  modified_base 1259: mod_base = OTHER; note = thymine
  modified_base 1266: mod_base = OTHER; note = thymine
  modified_base 1269..1270: mod_base = OTHER; note = thymine
  modified_base 1272..1273: mod_base = OTHER; note = thymine
  modified_base 1276..1277: mod_base = OTHER; note = thymine
  modified_base 1281: mod_base = OTHER; note = thymine
  modified_base 1291..1292: mod_base = OTHER; note = thymine
  modified_base 1296: mod_base = OTHER; note = thymine
  modified_base 1304: mod_base = OTHER; note = thymine
  modified_base 1320: mod_base = OTHER; note = thymine
  modified_base 1325: mod_base = OTHER; note = thymine
  modified_base 1328..1329: mod_base = OTHER; note = thymine
  modified_base 1337: mod_base = OTHER; note = thymine
  modified_base 1339..1340: mod_base = OTHER; note = thymine
  modified_base 1350..1351: mod_base = OTHER; note = thymine
  modified_base 1355: mod_base = OTHER; note = thymine
  modified_base 1371: mod_base = OTHER; note = thymine
  modified_base 1379: mod_base = OTHER; note = thymine
  modified_base 1384..1385: mod_base = OTHER; note = thymine
  modified_base 1388: mod_base = OTHER; note = thymine
  modified_base 1394..1395: mod_base = OTHER; note = thymine
  modified_base 1397: mod_base = OTHER; note = thymine
  modified_base 1400: mod_base = OTHER; note = thymine
  modified_base 1402..1405: mod_base = OTHER; note = thymine
  modified_base 1411: mod_base = OTHER; note = thymine
  modified_base 1413: mod_base = OTHER; note = thymine
  modified_base 1417: mod_base = OTHER; note = thymine
  modified_base 1419: mod_base = OTHER; note = thymine
  modified_base 1421: mod_base = OTHER; note = thymine
  modified_base 1424: mod_base = OTHER; note = thymine
  modified_base 1430: mod_base = OTHER; note = thymine
  modified_base 1432..1433: mod_base = OTHER; note = thymine
  modified_base 1436: mod_base = OTHER; note = thymine
  modified_base 1442..1443: mod_base = OTHER; note = thymine
  modified_base 1445: mod_base = OTHER; note = thymine
  modified_base 1451: mod_base = OTHER; note = thymine
  modified_base 1469: mod_base = OTHER; note = thymine
  modified_base 1476: mod_base = OTHER; note = thymine
  modified_base 1480: mod_base = OTHER; note = thymine
  modified_base 1483: mod_base = OTHER; note = thymine
  modified_base 1485: mod_base = OTHER; note = thymine
  modified_base 1501..1502: mod_base = OTHER; note = thymine
  modified_base 1517..1519: mod_base = OTHER; note = thymine
  modified_base 1521: mod_base = OTHER; note = thymine
  modified_base 1524..1525: mod_base = OTHER; note = thymine
  modified_base 1527: mod_base = OTHER; note = thymine
  modified_base 1530: mod_base = OTHER; note = thymine
  modified_base 1534..1536: mod_base = OTHER; note = thymine
  modified_base 1541: mod_base = OTHER; note = thymine
  modified_base 1545: mod_base = OTHER; note = thymine
  modified_base 1548: mod_base = OTHER; note = thymine
  modified_base 1550: mod_base = OTHER; note = thymine
  modified_base 1559: mod_base = OTHER; note = thymine
  modified_base 1561: mod_base = OTHER; note = thymine
  modified_base 1573: mod_base = OTHER; note = thymine
  modified_base 1587: mod_base = OTHER; note = thymine
  modified_base 1591: mod_base = OTHER; note = thymine
  modified_base 1595: mod_base = OTHER; note = thymine
  modified_base 1597: mod_base = OTHER; note = thymine
  modified_base 1603: mod_base = OTHER; note = thymine
  modified_base 1613: mod_base = OTHER; note = thymine
  modified_base 1620: mod_base = OTHER; note = thymine
  modified_base 1638: mod_base = OTHER; note = thymine
  modified_base 1640..1641: mod_base = OTHER; note = thymine
  modified_base 1646: mod_base = OTHER; note = thymine
  modified_base 1648: mod_base = OTHER; note = thymine
  modified_base 1656..1658: mod_base = OTHER; note = thymine
  modified_base 1664: mod_base = OTHER; note = thymine
  modified_base 1666: mod_base = OTHER; note = thymine
  modified_base 1672..1673: mod_base = OTHER; note = thymine
  modified_base 1677: mod_base = OTHER; note = thymine
  modified_base 1680: mod_base = OTHER; note = thymine
  modified_base 1682: mod_base = OTHER; note = thymine
  modified_base 1690: mod_base = OTHER; note = thymine
  modified_base 1692..1693: mod_base = OTHER; note = thymine
  modified_base 1695: mod_base = OTHER; note = thymine
  modified_base 1702..1705: mod_base = OTHER; note = thymine
  modified_base 1710: mod_base = OTHER; note = thymine
  modified_base 1712: mod_base = OTHER; note = thymine
  modified_base 1720..1721: mod_base = OTHER; note = thymine
  modified_base 1724..1726: mod_base = OTHER; note = thymine
  modified_base 1735..1737: mod_base = OTHER; note = thymine
  modified_base 1744: mod_base = OTHER; note = thymine
  modified_base 1747: mod_base = OTHER; note = thymine
  modified_base 1752: mod_base = OTHER; note = thymine
  modified_base 1756: mod_base = OTHER; note = thymine
  modified_base 1760: mod_base = OTHER; note = thymine
  modified_base 1767: mod_base = OTHER; note = thymine
  modified_base 1774: mod_base = OTHER; note = thymine
  modified_base 1777: mod_base = OTHER; note = thymine
  modified_base 1779: mod_base = OTHER; note = thymine
  modified_base 1781..1783: mod_base = OTHER; note = thymine
  modified_base 1785..1786: mod_base = OTHER; note = thymine
  modified_base 1788..1792: mod_base = OTHER; note = thymine
  modified_base 1794: mod_base = OTHER; note = thymine
  modified_base 1798..1799: mod_base = OTHER; note = thymine
  modified_base 1802: mod_base = OTHER; note = thymine
  modified_base 1804..1805: mod_base = OTHER; note = thymine
  modified_base 1810: mod_base = OTHER; note = thymine
  modified_base 1812: mod_base = OTHER; note = thymine
  modified_base 1816: mod_base = OTHER; note = thymine
  modified_base 1818..1819: mod_base = OTHER; note = thymine
  modified_base 1833: mod_base = OTHER; note = thymine
  modified_base 1835..1837: mod_base = OTHER; note = thymine
  modified_base 1839: mod_base = OTHER; note = thymine
  modified_base 1847..1849: mod_base = OTHER; note = thymine
  modified_base 1851: mod_base = OTHER; note = thymine
  modified_base 1853..1854: mod_base = OTHER; note = thymine
  modified_base 1857: mod_base = OTHER; note = thymine
  modified_base 1860: mod_base = OTHER; note = thymine
  modified_base 1864: mod_base = OTHER; note = thymine
  modified_base 1871..1872: mod_base = OTHER; note = thymine
  modified_base 1876: mod_base = OTHER; note = thymine
  modified_base 1879: mod_base = OTHER; note = thymine
  modified_base 1882..1884: mod_base = OTHER; note = thymine
  modified_base 1890: mod_base = OTHER; note = thymine
  modified_base 1894: mod_base = OTHER; note = thymine
  modified_base 1899..1900: mod_base = OTHER; note = thymine
  modified_base 1903: mod_base = OTHER; note = thymine
  modified_base 1905: mod_base = OTHER; note = thymine
  modified_base 1908..1909: mod_base = OTHER; note = thymine
  modified_base 1912: mod_base = OTHER; note = thymine
  modified_base 1914..1915: mod_base = OTHER; note = thymine
  modified_base 1917: mod_base = OTHER; note = thymine
  modified_base 1919: mod_base = OTHER; note = thymine
  modified_base 1923: mod_base = OTHER; note = thymine
  modified_base 1932..1934: mod_base = OTHER; note = thymine
  modified_base 1936: mod_base = OTHER; note = thymine
  modified_base 1939: mod_base = OTHER; note = thymine
  modified_base 1941: mod_base = OTHER; note = thymine
  modified_base 1944: mod_base = OTHER; note = thymine
  modified_base 1952..1953: mod_base = OTHER; note = thymine
  modified_base 1960..1961: mod_base = OTHER; note = thymine
  modified_base 1969: mod_base = OTHER; note = thymine
  modified_base 1974: mod_base = OTHER; note = thymine
  modified_base 1988: mod_base = OTHER; note = thymine
  modified_base 1990: mod_base = OTHER; note = thymine
  modified_base 1994: mod_base = OTHER; note = thymine
  modified_base 2002: mod_base = OTHER; note = thymine
  modified_base 2012..2013: mod_base = OTHER; note = thymine
  modified_base 2019..2020: mod_base = OTHER; note = thymine
  modified_base 2028: mod_base = OTHER; note = thymine
  modified_base 2037: mod_base = OTHER; note = thymine
  modified_base 2041: mod_base = OTHER; note = thymine
  modified_base 2046: mod_base = OTHER; note = thymine
  modified_base 2048: mod_base = OTHER; note = thymine
  modified_base 2052: mod_base = OTHER; note = thymine
  modified_base 2055..2056: mod_base = OTHER; note = thymine
  modified_base 2061: mod_base = OTHER; note = thymine
  modified_base 2063..2064: mod_base = OTHER; note = thymine
  modified_base 2067..2068: mod_base = OTHER; note = thymine
  modified_base 2072..2073: mod_base = OTHER; note = thymine
  modified_base 2075..2079: mod_base = OTHER; note = thymine
  modified_base 2082: mod_base = OTHER; note = thymine
  modified_base 2090..2091: mod_base = OTHER; note = thymine
  modified_base 2095: mod_base = OTHER; note = thymine
  modified_base 2097: mod_base = OTHER; note = thymine
  modified_base 2103: mod_base = OTHER; note = thymine
  modified_base 2105: mod_base = OTHER; note = thymine
  modified_base 2107: mod_base = OTHER; note = thymine
  modified_base 2112: mod_base = OTHER; note = thymine
  modified_base 2118..2119: mod_base = OTHER; note = thymine
  modified_base 2121: mod_base = OTHER; note = thymine
  modified_base 2123: mod_base = OTHER; note = thymine
  modified_base 2126..2127: mod_base = OTHER; note = thymine
  modified_base 2134..2135: mod_base = OTHER; note = thymine
  modified_base 2138: mod_base = OTHER; note = thymine
  modified_base 2140..2142: mod_base = OTHER; note = thymine
  modified_base 2147: mod_base = OTHER; note = thymine
  modified_base 2151: mod_base = OTHER; note = thymine
  modified_base 2154..2155: mod_base = OTHER; note = thymine
  modified_base 2159: mod_base = OTHER; note = thymine
  modified_base 2161: mod_base = OTHER; note = thymine
  modified_base 2166: mod_base = OTHER; note = thymine
  modified_base 2170: mod_base = OTHER; note = thymine
  modified_base 2172: mod_base = OTHER; note = thymine
  modified_base 2176: mod_base = OTHER; note = thymine
  modified_base 2180: mod_base = OTHER; note = thymine
  modified_base 2182: mod_base = OTHER; note = thymine
  modified_base 2190..2191: mod_base = OTHER; note = thymine
  modified_base 2196: mod_base = OTHER; note = thymine
  modified_base 2200..2201: mod_base = OTHER; note = thymine
  modified_base 2204: mod_base = OTHER; note = thymine
  modified_base 2209: mod_base = OTHER; note = thymine
  modified_base 2214: mod_base = OTHER; note = thymine
  modified_base 2216..2219: mod_base = OTHER; note = thymine
  modified_base 2227..2228: mod_base = OTHER; note = thymine
  modified_base 2230: mod_base = OTHER; note = thymine
  modified_base 2232: mod_base = OTHER; note = thymine
  modified_base 2236: mod_base = OTHER; note = thymine
  modified_base 2238: mod_base = OTHER; note = thymine
  modified_base 2242..2243: mod_base = OTHER; note = thymine
  modified_base 2253: mod_base = OTHER; note = thymine
  modified_base 2258: mod_base = OTHER; note = thymine
  modified_base 2264: mod_base = OTHER; note = thymine
  modified_base 2267: mod_base = OTHER; note = thymine
  modified_base 2270: mod_base = OTHER; note = thymine
  modified_base 2279: mod_base = OTHER; note = thymine
  modified_base 2284: mod_base = OTHER; note = thymine
  modified_base 2288: mod_base = OTHER; note = thymine
  modified_base 2292..2293: mod_base = OTHER; note = thymine
  modified_base 2297: mod_base = OTHER; note = thymine
  modified_base 2303..2305: mod_base = OTHER; note = thymine
  modified_base 2307: mod_base = OTHER; note = thymine
  modified_base 2311..2312: mod_base = OTHER; note = thymine
  modified_base 2314: mod_base = OTHER; note = thymine
  modified_base 2319: mod_base = OTHER; note = thymine
  modified_base 2323..2324: mod_base = OTHER; note = thymine
  modified_base 2326..2327: mod_base = OTHER; note = thymine
  modified_base 2329: mod_base = OTHER; note = thymine
  modified_base 2331..2334: mod_base = OTHER; note = thymine
  modified_base 2339: mod_base = OTHER; note = thymine
  modified_base 2342: mod_base = OTHER; note = thymine
  modified_base 2344: mod_base = OTHER; note = thymine
  modified_base 2353: mod_base = OTHER; note = thymine
  modified_base 2357: mod_base = OTHER; note = thymine
  modified_base 2359..2360: mod_base = OTHER; note = thymine
  modified_base 2362: mod_base = OTHER; note = thymine
  modified_base 2369: mod_base = OTHER; note = thymine
  modified_base 2371: mod_base = OTHER; note = thymine
  modified_base 2373: mod_base = OTHER; note = thymine
  modified_base 2375: mod_base = OTHER; note = thymine
  modified_base 2381: mod_base = OTHER; note = thymine
  modified_base 2384..2385: mod_base = OTHER; note = thymine
  modified_base 2390: mod_base = OTHER; note = thymine
  modified_base 2402..2404: mod_base = OTHER; note = thymine
  modified_base 2406: mod_base = OTHER; note = thymine
  modified_base 2413: mod_base = OTHER; note = thymine
  modified_base 2415..2418: mod_base = OTHER; note = thymine
  modified_base 2421: mod_base = OTHER; note = thymine
  modified_base 2427..2428: mod_base = OTHER; note = thymine
  modified_base 2431: mod_base = OTHER; note = thymine
  modified_base 2433: mod_base = OTHER; note = thymine
  modified_base 2441..2442: mod_base = OTHER; note = thymine
  modified_base 2445: mod_base = OTHER; note = thymine
  modified_base 2447: mod_base = OTHER; note = thymine
  modified_base 2458: mod_base = OTHER; note = thymine
  modified_base 2460..2461: mod_base = OTHER; note = thymine
  modified_base 2465..2468: mod_base = OTHER; note = thymine
  modified_base 2470: mod_base = OTHER; note = thymine
  modified_base 2472..2473: mod_base = OTHER; note = thymine
  modified_base 2486: mod_base = OTHER; note = thymine
  modified_base 2490..2491: mod_base = OTHER; note = thymine
  modified_base 2499: mod_base = OTHER; note = thymine
  modified_base 2502: mod_base = OTHER; note = thymine
  modified_base 2506: mod_base = OTHER; note = thymine
  modified_base 2508..2509: mod_base = OTHER; note = thymine
  modified_base 2512..2513: mod_base = OTHER; note = thymine
  modified_base 2517..2518: mod_base = OTHER; note = thymine
  modified_base 2521: mod_base = OTHER; note = thymine
  modified_base 2527..2528: mod_base = OTHER; note = thymine
  modified_base 2532: mod_base = OTHER; note = thymine
  modified_base 2534: mod_base = OTHER; note = thymine
  modified_base 2539: mod_base = OTHER; note = thymine
  modified_base 2541..2542: mod_base = OTHER; note = thymine
  modified_base 2550: mod_base = OTHER; note = thymine
  modified_base 2561: mod_base = OTHER; note = thymine
  modified_base 2567: mod_base = OTHER; note = thymine
  modified_base 2571..2572: mod_base = OTHER; note = thymine
  modified_base 2590: mod_base = OTHER; note = thymine
  modified_base 2595..2596: mod_base = OTHER; note = thymine
  modified_base 2598: mod_base = OTHER; note = thymine
  modified_base 2608: mod_base = OTHER; note = thymine
  modified_base 2610..2611: mod_base = OTHER; note = thymine
  modified_base 2614: mod_base = OTHER; note = thymine
  modified_base 2617: mod_base = OTHER; note = thymine
  modified_base 2622: mod_base = OTHER; note = thymine
  modified_base 2625: mod_base = OTHER; note = thymine
  modified_base 2627..2628: mod_base = OTHER; note = thymine
  modified_base 2632: mod_base = OTHER; note = thymine
  modified_base 2635: mod_base = OTHER; note = thymine
  modified_base 2648: mod_base = OTHER; note = thymine
  modified_base 2652: mod_base = OTHER; note = thymine
  modified_base 2655..2656: mod_base = OTHER; note = thymine
  modified_base 2658: mod_base = OTHER; note = thymine
  modified_base 2660..2662: mod_base = OTHER; note = thymine
  modified_base 2668..2670: mod_base = OTHER; note = thymine
  modified_base 2676: mod_base = OTHER; note = thymine
  modified_base 2679..2680: mod_base = OTHER; note = thymine
  modified_base 2685..2687: mod_base = OTHER; note = thymine
  modified_base 2691: mod_base = OTHER; note = thymine
  modified_base 2694..2695: mod_base = OTHER; note = thymine
  modified_base 2699: mod_base = OTHER; note = thymine
  modified_base 2701: mod_base = OTHER; note = thymine
  modified_base 2703: mod_base = OTHER; note = thymine
  modified_base 2718: mod_base = OTHER; note = thymine
  modified_base 2720: mod_base = OTHER; note = thymine
  modified_base 2722: mod_base = OTHER; note = thymine
  modified_base 2729..2730: mod_base = OTHER; note = thymine
  modified_base 2732: mod_base = OTHER; note = thymine
  modified_base 2739: mod_base = OTHER; note = thymine
  modified_base 2742..2743: mod_base = OTHER; note = thymine
  modified_base 2752: mod_base = OTHER; note = thymine
  modified_base 2754: mod_base = OTHER; note = thymine
  modified_base 2756: mod_base = OTHER; note = thymine
  modified_base 2760: mod_base = OTHER; note = thymine
  modified_base 2762..2764: mod_base = OTHER; note = thymine
  modified_base 2770: mod_base = OTHER; note = thymine
  modified_base 2779: mod_base = OTHER; note = thymine
  modified_base 2782..2783: mod_base = OTHER; note = thymine
  modified_base 2788..2789: mod_base = OTHER; note = thymine
  modified_base 2794..2795: mod_base = OTHER; note = thymine
  modified_base 2797: mod_base = OTHER; note = thymine
  modified_base 2804..2805: mod_base = OTHER; note = thymine
  modified_base 2808..2809: mod_base = OTHER; note = thymine
  modified_base 2811..2812: mod_base = OTHER; note = thymine
  modified_base 2819: mod_base = OTHER; note = thymine
  modified_base 2822: mod_base = OTHER; note = thymine
  modified_base 2826: mod_base = OTHER; note = thymine
  modified_base 2828..2830: mod_base = OTHER; note = thymine
  modified_base 2835: mod_base = OTHER; note = thymine
  modified_base 2843: mod_base = OTHER; note = thymine
  modified_base 2845..2846: mod_base = OTHER; note = thymine
  modified_base 2857..2858: mod_base = OTHER; note = thymine
  modified_base 2865: mod_base = OTHER; note = thymine
  modified_base 2868: mod_base = OTHER; note = thymine
  modified_base 2874..2876: mod_base = OTHER; note = thymine
  modified_base 2878: mod_base = OTHER; note = thymine
  modified_base 2881: mod_base = OTHER; note = thymine
  modified_base 2888: mod_base = OTHER; note = thymine
  modified_base 2890: mod_base = OTHER; note = thymine
  modified_base 2892: mod_base = OTHER; note = thymine
  modified_base 2894: mod_base = OTHER; note = thymine
  modified_base 2899: mod_base = OTHER; note = thymine
  modified_base 2901: mod_base = OTHER; note = thymine
  modified_base 2906: mod_base = OTHER; note = thymine
  modified_base 2908..2910: mod_base = OTHER; note = thymine
  modified_base 2913: mod_base = OTHER; note = thymine
  modified_base 2915: mod_base = OTHER; note = thymine
  modified_base 2918..2919: mod_base = OTHER; note = thymine
  modified_base 2922: mod_base = OTHER; note = thymine
  modified_base 2924: mod_base = OTHER; note = thymine
  modified_base 2926..2927: mod_base = OTHER; note = thymine
  modified_base 2929..2930: mod_base = OTHER; note = thymine
  modified_base 2934..2935: mod_base = OTHER; note = thymine
  modified_base 2941: mod_base = OTHER; note = thymine
  modified_base 2943: mod_base = OTHER; note = thymine
  modified_base 2945: mod_base = OTHER; note = thymine
  modified_base 2951: mod_base = OTHER; note = thymine
  modified_base 2958: mod_base = OTHER; note = thymine
  modified_base 2965..2967: mod_base = OTHER; note = thymine
  modified_base 2974: mod_base = OTHER; note = thymine
  modified_base 2977: mod_base = OTHER; note = thymine
  modified_base 2980: mod_base = OTHER; note = thymine
  modified_base 2982..2983: mod_base = OTHER; note = thymine
  modified_base 2988..2989: mod_base = OTHER; note = thymine
  modified_base 2994..2995: mod_base = OTHER; note = thymine
  modified_base 2997: mod_base = OTHER; note = thymine
  modified_base 3003: mod_base = OTHER; note = thymine
  modified_base 3019: mod_base = OTHER; note = thymine
  modified_base 3023..3024: mod_base = OTHER; note = thymine
  modified_base 3026..3027: mod_base = OTHER; note = thymine
  modified_base 3029: mod_base = OTHER; note = thymine
  modified_base 3034: mod_base = OTHER; note = thymine
  modified_base 3040: mod_base = OTHER; note = thymine
  modified_base 3047..3049: mod_base = OTHER; note = thymine
  modified_base 3057..3058: mod_base = OTHER; note = thymine
  modified_base 3060..3061: mod_base = OTHER; note = thymine
  modified_base 3063..3065: mod_base = OTHER; note = thymine
  modified_base 3068: mod_base = OTHER; note = thymine
  modified_base 3070..3071: mod_base = OTHER; note = thymine
  modified_base 3075..3077: mod_base = OTHER; note = thymine
  modified_base 3081: mod_base = OTHER; note = thymine
  modified_base 3093: mod_base = OTHER; note = thymine
  modified_base 3095: mod_base = OTHER; note = thymine
  modified_base 3100: mod_base = OTHER; note = thymine
  modified_base 3102: mod_base = OTHER; note = thymine
  modified_base 3105..3106: mod_base = OTHER; note = thymine
  modified_base 3112..3113: mod_base = OTHER; note = thymine
  modified_base 3115: mod_base = OTHER; note = thymine
  modified_base 3119: mod_base = OTHER; note = thymine
  modified_base 3121: mod_base = OTHER; note = thymine
  modified_base 3123: mod_base = OTHER; note = thymine
  modified_base 3125: mod_base = OTHER; note = thymine
  modified_base 3129: mod_base = OTHER; note = thymine
  modified_base 3131..3133: mod_base = OTHER; note = thymine
  modified_base 3135: mod_base = OTHER; note = thymine
  modified_base 3138: mod_base = OTHER; note = thymine
  modified_base 3140: mod_base = OTHER; note = thymine
  modified_base 3142: mod_base = OTHER; note = thymine
  modified_base 3144..3147: mod_base = OTHER; note = thymine
  modified_base 3150..3151: mod_base = OTHER; note = thymine
  modified_base 3154: mod_base = OTHER; note = thymine
  modified_base 3157: mod_base = OTHER; note = thymine
  modified_base 3160: mod_base = OTHER; note = thymine
  modified_base 3163: mod_base = OTHER; note = thymine
  modified_base 3165: mod_base = OTHER; note = thymine
  modified_base 3171..3172: mod_base = OTHER; note = thymine
  modified_base 3175: mod_base = OTHER; note = thymine
  modified_base 3180: mod_base = OTHER; note = thymine
  modified_base 3189: mod_base = OTHER; note = thymine
  modified_base 3198..3199: mod_base = OTHER; note = thymine
  modified_base 3207..3208: mod_base = OTHER; note = thymine
  modified_base 3211..3212: mod_base = OTHER; note = thymine
  modified_base 3216: mod_base = OTHER; note = thymine
  modified_base 3219..3220: mod_base = OTHER; note = thymine
  modified_base 3223: mod_base = OTHER; note = thymine
  modified_base 3234..3235: mod_base = OTHER; note = thymine
  modified_base 3240: mod_base = OTHER; note = thymine
  modified_base 3242: mod_base = OTHER; note = thymine
  modified_base 3245: mod_base = OTHER; note = thymine
  modified_base 3247: mod_base = OTHER; note = thymine
  modified_base 3253: mod_base = OTHER; note = thymine
  modified_base 3255: mod_base = OTHER; note = thymine
  modified_base 3260..3261: mod_base = OTHER; note = thymine
  modified_base 3263: mod_base = OTHER; note = thymine
  modified_base 3265..3266: mod_base = OTHER; note = thymine
  modified_base 3268: mod_base = OTHER; note = thymine
  modified_base 3272: mod_base = OTHER; note = thymine
  modified_base 3275..3276: mod_base = OTHER; note = thymine
  modified_base 3280: mod_base = OTHER; note = thymine
  modified_base 3284..3285: mod_base = OTHER; note = thymine
  modified_base 3292: mod_base = OTHER; note = thymine
  modified_base 3294..3295: mod_base = OTHER; note = thymine
  modified_base 3299..3300: mod_base = OTHER; note = thymine
  modified_base 3303..3304: mod_base = OTHER; note = thymine
  modified_base 3316: mod_base = OTHER; note = thymine
  modified_base 3318..3319: mod_base = OTHER; note = thymine
  modified_base 3321..3322: mod_base = OTHER; note = thymine
  modified_base 3325: mod_base = OTHER; note = thymine
  modified_base 3329: mod_base = OTHER; note = thymine
  modified_base 3332..3334: mod_base = OTHER; note = thymine
  modified_base 3336: mod_base = OTHER; note = thymine
  modified_base 3346: mod_base = OTHER; note = thymine
  modified_base 3353: mod_base = OTHER; note = thymine
  modified_base 3358: mod_base = OTHER; note = thymine
  modified_base 3362: mod_base = OTHER; note = thymine
  modified_base 3367: mod_base = OTHER; note = thymine
  modified_base 3372: mod_base = OTHER; note = thymine
  modified_base 3374: mod_base = OTHER; note = thymine
  misc_feature 1..3377: note = Lymphocytic choriomeningitis mammarenavirus Viruses (LCMV); Lymphocytic choriomeningitis virus clone 13 segment S, complete sequence.ACCESSION DQ361065-VERSION DQ361065.2-GI:116563461.-ssRNA viruses; ssRNA negative-strand viruses; Arenaviridae; Mammarenavirus.
  source 1..3377: mol_type = other RNA
gcgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcctcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgaccaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 6 - RNA, 7229 nt, unidentified
  misc_feature 1..7229: note = Lymphocytic choriomeningitis mammarenavirus Viruses (LCMV); Lymphocytic choriomeningitis virus clone 13 segment L, complete sequence.-ACCESSION DQ361066-VERSION DQ361066.1-GI:86440167.-ssRNA viruses; ssRNA negative-strand viruses; Arenaviridae; Mammarenavirus
  source 1..7229: mol_type = other RNA
  modified_base 13: mod_base = OTHER; note = thymine
  modified_base 16: mod_base = OTHER; note = thymine
  modified_base 22..24: mod_base = OTHER; note = thymine
  modified_base 27..28: mod_base = OTHER; note = thymine
  modified_base 33: mod_base = OTHER; note = thymine
  modified_base 35..37: mod_base = OTHER; note = thymine
  modified_base 40..41: mod_base = OTHER; note = thymine
  modified_base 49..51: mod_base = OTHER; note = thymine
  modified_base 53..54: mod_base = OTHER; note = thymine
  modified_base 57: mod_base = OTHER; note = thymine
  modified_base 63: mod_base = OTHER; note = thymine
  modified_base 65: mod_base = OTHER; note = thymine
  modified_base 72: mod_base = OTHER; note = thymine
  modified_base 78: mod_base = OTHER; note = thymine
  modified_base 82: mod_base = OTHER; note = thymine
  modified_base 85: mod_base = OTHER; note = thymine
  modified_base 91: mod_base = OTHER; note = thymine
  modified_base 95: mod_base = OTHER; note = thymine
  modified_base 105: mod_base = OTHER; note = thymine
  modified_base 128: mod_base = OTHER; note = thymine
  modified_base 131: mod_base = OTHER; note = thymine
  modified_base 148: mod_base = OTHER; note = thymine
  modified_base 151: mod_base = OTHER; note = thymine
  modified_base 158: mod_base = OTHER; note = thymine
  modified_base 165: mod_base = OTHER; note = thymine
  modified_base 167: mod_base = OTHER; note = thymine
  modified_base 169..170: mod_base = OTHER; note = thymine
  modified_base 176..178: mod_base = OTHER; note = thymine
  modified_base 183: mod_base = OTHER; note = thymine
  modified_base 189: mod_base = OTHER; note = thymine
  modified_base 191..192: mod_base = OTHER; note = thymine
  modified_base 195: mod_base = OTHER; note = thymine
  modified_base 204..206: mod_base = OTHER; note = thymine
  modified_base 213..214: mod_base = OTHER; note = thymine
  modified_base 217: mod_base = OTHER; note = thymine
  modified_base 222: mod_base = OTHER; note = thymine
  modified_base 227: mod_base = OTHER; note = thymine
  modified_base 234: mod_base = OTHER; note = thymine
  modified_base 238..240: mod_base = OTHER; note = thymine
  modified_base 249: mod_base = OTHER; note = thymine
  modified_base 251..253: mod_base = OTHER; note = thymine
  modified_base 259..260: mod_base = OTHER; note = thymine
  modified_base 262: mod_base = OTHER; note = thymine
  modified_base 265: mod_base = OTHER; note = thymine
  modified_base 267: mod_base = OTHER; note = thymine
  modified_base 271: mod_base = OTHER; note = thymine
  modified_base 273: mod_base = OTHER; note = thymine
  modified_base 282: mod_base = OTHER; note = thymine
  modified_base 284: mod_base = OTHER; note = thymine
  modified_base 287: mod_base = OTHER; note = thymine
  modified_base 289..291: mod_base = OTHER; note = thymine
  modified_base 293: mod_base = OTHER; note = thymine
  modified_base 297: mod_base = OTHER; note = thymine
  modified_base 299: mod_base = OTHER; note = thymine
  modified_base 303..304: mod_base = OTHER; note = thymine
  modified_base 315..316: mod_base = OTHER; note = thymine
  modified_base 322: mod_base = OTHER; note = thymine
  modified_base 324: mod_base = OTHER; note = thymine
  modified_base 339: mod_base = OTHER; note = thymine
  modified_base 341: mod_base = OTHER; note = thymine
  modified_base 347: mod_base = OTHER; note = thymine
  modified_base 351: mod_base = OTHER; note = thymine
  modified_base 360: mod_base = OTHER; note = thymine
  modified_base 368: mod_base = OTHER; note = thymine
  modified_base 414: mod_base = OTHER; note = thymine
  modified_base 559: mod_base = OTHER; note = thymine
  modified_base 565: mod_base = OTHER; note = thymine
  modified_base 569: mod_base = OTHER; note = thymine
  modified_base 573: mod_base = OTHER; note = thymine
  modified_base 575: mod_base = OTHER; note = thymine
  modified_base 578: mod_base = OTHER; note = thymine
  modified_base 602: mod_base = OTHER; note = thymine
  modified_base 605: mod_base = OTHER; note = thymine
  modified_base 615: mod_base = OTHER; note = thymine
  modified_base 620..621: mod_base = OTHER; note = thymine
  modified_base 625: mod_base = OTHER; note = thymine
  modified_base 627: mod_base = OTHER; note = thymine
  modified_base 633: mod_base = OTHER; note = thymine
  modified_base 643: mod_base = OTHER; note = thymine
  modified_base 645..647: mod_base = OTHER; note = thymine
  modified_base 650: mod_base = OTHER; note = thymine
  modified_base 654: mod_base = OTHER; note = thymine
  modified_base 660: mod_base = OTHER; note = thymine
  modified_base 663: mod_base = OTHER; note = thymine
  modified_base 666: mod_base = OTHER; note = thymine
  modified_base 670..672: mod_base = OTHER; note = thymine
  modified_base 675: mod_base = OTHER; note = thymine
  modified_base 685: mod_base = OTHER; note = thymine
  modified_base 687: mod_base = OTHER; note = thymine
  modified_base 692: mod_base = OTHER; note = thymine
  modified_base 695..696: mod_base = OTHER; note = thymine
  modified_base 710: mod_base = OTHER; note = thymine
  modified_base 712: mod_base = OTHER; note = thymine
  modified_base 727: mod_base = OTHER; note = thymine
  modified_base 732: mod_base = OTHER; note = thymine
  modified_base 741: mod_base = OTHER; note = thymine
  modified_base 745: mod_base = OTHER; note = thymine
  modified_base 747: mod_base = OTHER; note = thymine
  modified_base 749: mod_base = OTHER; note = thymine
  modified_base 751: mod_base = OTHER; note = thymine
  modified_base 753: mod_base = OTHER; note = thymine
  modified_base 757: mod_base = OTHER; note = thymine
  modified_base 760..761: mod_base = OTHER; note = thymine
  modified_base 764: mod_base = OTHER; note = thymine
  modified_base 771..772: mod_base = OTHER; note = thymine
  modified_base 777..778: mod_base = OTHER; note = thymine
  modified_base 780: mod_base = OTHER; note = thymine
  modified_base 782: mod_base = OTHER; note = thymine
  modified_base 784: mod_base = OTHER; note = thymine
  modified_base 797: mod_base = OTHER; note = thymine
  modified_base 799..800: mod_base = OTHER; note = thymine
  modified_base 807..808: mod_base = OTHER; note = thymine
  modified_base 810: mod_base = OTHER; note = thymine
  modified_base 814: mod_base = OTHER; note = thymine
  modified_base 818: mod_base = OTHER; note = thymine
  modified_base 830..831: mod_base = OTHER; note = thymine
  modified_base 842: mod_base = OTHER; note = thymine
  modified_base 846: mod_base = OTHER; note = thymine
  modified_base 849: mod_base = OTHER; note = thymine
  modified_base 851..854: mod_base = OTHER; note = thymine
  modified_base 857: mod_base = OTHER; note = thymine
  modified_base 863: mod_base = OTHER; note = thymine
  modified_base 868: mod_base = OTHER; note = thymine
  modified_base 871: mod_base = OTHER; note = thymine
  modified_base 874: mod_base = OTHER; note = thymine
  modified_base 883..885: mod_base = OTHER; note = thymine
  modified_base 888: mod_base = OTHER; note = thymine
  modified_base 890: mod_base = OTHER; note = thymine
  modified_base 893..894: mod_base = OTHER; note = thymine
  modified_base 897: mod_base = OTHER; note = thymine
  modified_base 899: mod_base = OTHER; note = thymine
  modified_base 905..906: mod_base = OTHER; note = thymine
  modified_base 912: mod_base = OTHER; note = thymine
  modified_base 915: mod_base = OTHER; note = thymine
  modified_base 917..920: mod_base = OTHER; note = thymine
  modified_base 929..931: mod_base = OTHER; note = thymine
  modified_base 933..934: mod_base = OTHER; note = thymine
  modified_base 937..938: mod_base = OTHER; note = thymine
  modified_base 954: mod_base = OTHER; note = thymine
  modified_base 956..958: mod_base = OTHER; note = thymine
  modified_base 963: mod_base = OTHER; note = thymine
  modified_base 967: mod_base = OTHER; note = thymine
  modified_base 977: mod_base = OTHER; note = thymine
  modified_base 983: mod_base = OTHER; note = thymine
  modified_base 988: mod_base = OTHER; note = thymine
  modified_base 994: mod_base = OTHER; note = thymine
  modified_base 996: mod_base = OTHER; note = thymine
  modified_base 998: mod_base = OTHER; note = thymine
  modified_base 1004: mod_base = OTHER; note = thymine
  modified_base 1008: mod_base = OTHER; note = thymine
  modified_base 1012: mod_base = OTHER; note = thymine
  modified_base 1014: mod_base = OTHER; note = thymine
  modified_base 1017: mod_base = OTHER; note = thymine
  modified_base 1022: mod_base = OTHER; note = thymine
  modified_base 1027: mod_base = OTHER; note = thymine
  modified_base 1029..1030: mod_base = OTHER; note = thymine
  modified_base 1032..1035: mod_base = OTHER; note = thymine
  modified_base 1049: mod_base = OTHER; note = thymine
  modified_base 1055: mod_base = OTHER; note = thymine
  modified_base 1064: mod_base = OTHER; note = thymine
  modified_base 1072: mod_base = OTHER; note = thymine
  modified_base 1074: mod_base = OTHER; note = thymine
  modified_base 1082: mod_base = OTHER; note = thymine
  modified_base 1084..1085: mod_base = OTHER; note = thymine
  modified_base 1089: mod_base = OTHER; note = thymine
  modified_base 1091: mod_base = OTHER; note = thymine
  modified_base 1094: mod_base = OTHER; note = thymine
  modified_base 1097: mod_base = OTHER; note = thymine
  modified_base 1103: mod_base = OTHER; note = thymine
  modified_base 1105..1106: mod_base = OTHER; note = thymine
  modified_base 1111: mod_base = OTHER; note = thymine
  modified_base 1113: mod_base = OTHER; note = thymine
  modified_base 1116: mod_base = OTHER; note = thymine
  modified_base 1118: mod_base = OTHER; note = thymine
  modified_base 1130: mod_base = OTHER; note = thymine
  modified_base 1133..1134: mod_base = OTHER; note = thymine
  modified_base 1139..1140: mod_base = OTHER; note = thymine
  modified_base 1151: mod_base = OTHER; note = thymine
  modified_base 1153: mod_base = OTHER; note = thymine
  modified_base 1155: mod_base = OTHER; note = thymine
  modified_base 1158: mod_base = OTHER; note = thymine
  modified_base 1162: mod_base = OTHER; note = thymine
  modified_base 1166: mod_base = OTHER; note = thymine
  modified_base 1174..1175: mod_base = OTHER; note = thymine
  modified_base 1178: mod_base = OTHER; note = thymine
  modified_base 1185..1186: mod_base = OTHER; note = thymine
  modified_base 1188..1189: mod_base = OTHER; note = thymine
  modified_base 1191: mod_base = OTHER; note = thymine
  modified_base 1193: mod_base = OTHER; note = thymine
  modified_base 1195: mod_base = OTHER; note = thymine
  modified_base 1197: mod_base = OTHER; note = thymine
  modified_base 1199: mod_base = OTHER; note = thymine
  modified_base 1205..1206: mod_base = OTHER; note = thymine
  modified_base 1214: mod_base = OTHER; note = thymine
  modified_base 1222: mod_base = OTHER; note = thymine
  modified_base 1225: mod_base = OTHER; note = thymine
  modified_base 1231..1232: mod_base = OTHER; note = thymine
  modified_base 1243: mod_base = OTHER; note = thymine
  modified_base 1247..1248: mod_base = OTHER; note = thymine
  modified_base 1250: mod_base = OTHER; note = thymine
  modified_base 1252: mod_base = OTHER; note = thymine
  modified_base 1255..1256: mod_base = OTHER; note = thymine
  modified_base 1263: mod_base = OTHER; note = thymine
  modified_base 1273: mod_base = OTHER; note = thymine
  modified_base 1275: mod_base = OTHER; note = thymine
  modified_base 1279: mod_base = OTHER; note = thymine
  modified_base 1285..1287: mod_base = OTHER; note = thymine
  modified_base 1290..1291: mod_base = OTHER; note = thymine
  modified_base 1298: mod_base = OTHER; note = thymine
  modified_base 1302: mod_base = OTHER; note = thymine
  modified_base 1304..1305: mod_base = OTHER; note = thymine
  modified_base 1307..1308: mod_base = OTHER; note = thymine
  modified_base 1310: mod_base = OTHER; note = thymine
  modified_base 1313..1314: mod_base = OTHER; note = thymine
  modified_base 1316: mod_base = OTHER; note = thymine
  modified_base 1320: mod_base = OTHER; note = thymine
  modified_base 1325: mod_base = OTHER; note = thymine
  modified_base 1330: mod_base = OTHER; note = thymine
  modified_base 1335..1336: mod_base = OTHER; note = thymine
  modified_base 1343: mod_base = OTHER; note = thymine
  modified_base 1348: mod_base = OTHER; note = thymine
  modified_base 1354..1357: mod_base = OTHER; note = thymine
  modified_base 1359: mod_base = OTHER; note = thymine
  modified_base 1361: mod_base = OTHER; note = thymine
  modified_base 1364..1365: mod_base = OTHER; note = thymine
  modified_base 1371: mod_base = OTHER; note = thymine
  modified_base 1373: mod_base = OTHER; note = thymine
  modified_base 1376: mod_base = OTHER; note = thymine
  modified_base 1385: mod_base = OTHER; note = thymine
  modified_base 1388..1390: mod_base = OTHER; note = thymine
  modified_base 1396..1398: mod_base = OTHER; note = thymine
  modified_base 1402: mod_base = OTHER; note = thymine
  modified_base 1406..1408: mod_base = OTHER; note = thymine
  modified_base 1412..1414: mod_base = OTHER; note = thymine
  modified_base 1416: mod_base = OTHER; note = thymine
  modified_base 1424: mod_base = OTHER; note = thymine
  modified_base 1428: mod_base = OTHER; note = thymine
  modified_base 1431: mod_base = OTHER; note = thymine
  modified_base 1437: mod_base = OTHER; note = thymine
  modified_base 1445..1446: mod_base = OTHER; note = thymine
  modified_base 1457: mod_base = OTHER; note = thymine
  modified_base 1459: mod_base = OTHER; note = thymine
  modified_base 1464..1465: mod_base = OTHER; note = thymine
  modified_base 1469: mod_base = OTHER; note = thymine
  modified_base 1493: mod_base = OTHER; note = thymine
  modified_base 1499: mod_base = OTHER; note = thymine
  modified_base 1502..1503: mod_base = OTHER; note = thymine
  modified_base 1506: mod_base = OTHER; note = thymine
  modified_base 1512: mod_base = OTHER; note = thymine
  modified_base 1518: mod_base = OTHER; note = thymine
  modified_base 1520: mod_base = OTHER; note = thymine
  modified_base 1526..1530: mod_base = OTHER; note = thymine
  modified_base 1532..1533: mod_base = OTHER; note = thymine
  modified_base 1541..1543: mod_base = OTHER; note = thymine
  modified_base 1552: mod_base = OTHER; note = thymine
  modified_base 1555..1556: mod_base = OTHER; note = thymine
  modified_base 1570: mod_base = OTHER; note = thymine
  modified_base 1574: mod_base = OTHER; note = thymine
  modified_base 1583: mod_base = OTHER; note = thymine
  modified_base 1585..1586: mod_base = OTHER; note = thymine
  modified_base 1591: mod_base = OTHER; note = thymine
  modified_base 1596: mod_base = OTHER; note = thymine
  modified_base 1599..1601: mod_base = OTHER; note = thymine
  modified_base 1603..1605: mod_base = OTHER; note = thymine
  modified_base 1607: mod_base = OTHER; note = thymine
  modified_base 1618: mod_base = OTHER; note = thymine
  modified_base 1625..1626: mod_base = OTHER; note = thymine
  modified_base 1630: mod_base = OTHER; note = thymine
  modified_base 1633: mod_base = OTHER; note = thymine
  modified_base 1635: mod_base = OTHER; note = thymine
  modified_base 1638: mod_base = OTHER; note = thymine
  modified_base 1641..1642: mod_base = OTHER; note = thymine
  modified_base 1644..1645: mod_base = OTHER; note = thymine
  modified_base 1654..1655: mod_base = OTHER; note = thymine
  modified_base 1657: mod_base = OTHER; note = thymine
  modified_base 1666: mod_base = OTHER; note = thymine
  modified_base 1669..1670: mod_base = OTHER; note = thymine
  modified_base 1676: mod_base = OTHER; note = thymine
  modified_base 1682: mod_base = OTHER; note = thymine
  modified_base 1684..1687: mod_base = OTHER; note = thymine
  modified_base 1694..1695: mod_base = OTHER; note = thymine
  modified_base 1697..1698: mod_base = OTHER; note = thymine
  modified_base 1706: mod_base = OTHER; note = thymine
  modified_base 1710: mod_base = OTHER; note = thymine
  modified_base 1713: mod_base = OTHER; note = thymine
  modified_base 1716: mod_base = OTHER; note = thymine
  modified_base 1726..1727: mod_base = OTHER; note = thymine
  modified_base 1730: mod_base = OTHER; note = thymine
  modified_base 1740: mod_base = OTHER; note = thymine
  modified_base 1745: mod_base = OTHER; note = thymine
  modified_base 1750: mod_base = OTHER; note = thymine
  modified_base 1754: mod_base = OTHER; note = thymine
  modified_base 1757: mod_base = OTHER; note = thymine
  modified_base 1763..1764: mod_base = OTHER; note = thymine
  modified_base 1772: mod_base = OTHER; note = thymine
  modified_base 1788: mod_base = OTHER; note = thymine
  modified_base 1790: mod_base = OTHER; note = thymine
  modified_base 1793: mod_base = OTHER; note = thymine
  modified_base 1795..1796: mod_base = OTHER; note = thymine
  modified_base 1799: mod_base = OTHER; note = thymine
  modified_base 1803..1806: mod_base = OTHER; note = thymine
  modified_base 1811..1812: mod_base = OTHER; note = thymine
  modified_base 1820: mod_base = OTHER; note = thymine
  modified_base 1827: mod_base = OTHER; note = thymine
  modified_base 1831: mod_base = OTHER; note = thymine
  modified_base 1840..1841: mod_base = OTHER; note = thymine
  modified_base 1843: mod_base = OTHER; note = thymine
  modified_base 1847..1848: mod_base = OTHER; note = thymine
  modified_base 1851: mod_base = OTHER; note = thymine
  modified_base 1853: mod_base = OTHER; note = thymine
  modified_base 1857..1858: mod_base = OTHER; note = thymine
  modified_base 1871: mod_base = OTHER; note = thymine
  modified_base 1879..1881: mod_base = OTHER; note = thymine
  modified_base 1884..1885: mod_base = OTHER; note = thymine
  modified_base 1887: mod_base = OTHER; note = thymine
  modified_base 1893: mod_base = OTHER; note = thymine
  modified_base 1899: mod_base = OTHER; note = thymine
  modified_base 1902: mod_base = OTHER; note = thymine
  modified_base 1904..1909: mod_base = OTHER; note = thymine
  modified_base 1914: mod_base = OTHER; note = thymine
  modified_base 1917: mod_base = OTHER; note = thymine
  modified_base 1919: mod_base = OTHER; note = thymine
  modified_base 1926: mod_base = OTHER; note = thymine
  modified_base 1930: mod_base = OTHER; note = thymine
  modified_base 1940: mod_base = OTHER; note = thymine
  modified_base 1943: mod_base = OTHER; note = thymine
  modified_base 1946: mod_base = OTHER; note = thymine
  modified_base 1953: mod_base = OTHER; note = thymine
  modified_base 1955..1956: mod_base = OTHER; note = thymine
  modified_base 1961: mod_base = OTHER; note = thymine
  modified_base 1963: mod_base = OTHER; note = thymine
  modified_base 1971: mod_base = OTHER; note = thymine
  modified_base 1978..1979: mod_base = OTHER; note = thymine
  modified_base 1983: mod_base = OTHER; note = thymine
  modified_base 1987..1988: mod_base = OTHER; note = thymine
  modified_base 1991..1992: mod_base = OTHER; note = thymine
  modified_base 1997..1998: mod_base = OTHER; note = thymine
  modified_base 2002..2004: mod_base = OTHER; note = thymine
  modified_base 2006: mod_base = OTHER; note = thymine
  modified_base 2009: mod_base = OTHER; note = thymine
  modified_base 2014: mod_base = OTHER; note = thymine
  modified_base 2019..2020: mod_base = OTHER; note = thymine
  modified_base 2025: mod_base = OTHER; note = thymine
  modified_base 2027: mod_base = OTHER; note = thymine
  modified_base 2033..2035: mod_base = OTHER; note = thymine
  modified_base 2037..2038: mod_base = OTHER; note = thymine
  modified_base 2041: mod_base = OTHER; note = thymine
  modified_base 2045: mod_base = OTHER; note = thymine
  modified_base 2048: mod_base = OTHER; note = thymine
  modified_base 2053: mod_base = OTHER; note = thymine
  modified_base 2057..2059: mod_base = OTHER; note = thymine
  modified_base 2063: mod_base = OTHER; note = thymine
  modified_base 2066..2067: mod_base = OTHER; note = thymine
  modified_base 2073..2074: mod_base = OTHER; note = thymine
  modified_base 2078..2079: mod_base = OTHER; note = thymine
  modified_base 2081: mod_base = OTHER; note = thymine
  modified_base 2085: mod_base = OTHER; note = thymine
  modified_base 2087..2088: mod_base = OTHER; note = thymine
  modified_base 2090..2091: mod_base = OTHER; note = thymine
  modified_base 2093..2094: mod_base = OTHER; note = thymine
  modified_base 2098..2100: mod_base = OTHER; note = thymine
  modified_base 2103: mod_base = OTHER; note = thymine
  modified_base 2107..2108: mod_base = OTHER; note = thymine
  modified_base 2110: mod_base = OTHER; note = thymine
  modified_base 2113: mod_base = OTHER; note = thymine
  modified_base 2132: mod_base = OTHER; note = thymine
  modified_base 2141..2142: mod_base = OTHER; note = thymine
  modified_base 2145: mod_base = OTHER; note = thymine
  modified_base 2149: mod_base = OTHER; note = thymine
  modified_base 2154: mod_base = OTHER; note = thymine
  modified_base 2160: mod_base = OTHER; note = thymine
  modified_base 2162: mod_base = OTHER; note = thymine
  modified_base 2165: mod_base = OTHER; note = thymine
  modified_base 2181..2182: mod_base = OTHER; note = thymine
  modified_base 2191: mod_base = OTHER; note = thymine
  modified_base 2193: mod_base = OTHER; note = thymine
  modified_base 2195: mod_base = OTHER; note = thymine
  modified_base 2198: mod_base = OTHER; note = thymine
  modified_base 2204: mod_base = OTHER; note = thymine
  modified_base 2209..2210: mod_base = OTHER; note = thymine
  modified_base 2212: mod_base = OTHER; note = thymine
  modified_base 2216: mod_base = OTHER; note = thymine
  modified_base 2222: mod_base = OTHER; note = thymine
  modified_base 2224..2226: mod_base = OTHER; note = thymine
  modified_base 2228: mod_base = OTHER; note = thymine
  modified_base 2235: mod_base = OTHER; note = thymine
  modified_base 2237: mod_base = OTHER; note = thymine
  modified_base 2244..2245: mod_base = OTHER; note = thymine
  modified_base 2247: mod_base = OTHER; note = thymine
  modified_base 2252: mod_base = OTHER; note = thymine
  modified_base 2258..2259: mod_base = OTHER; note = thymine
  modified_base 2266: mod_base = OTHER; note = thymine
  modified_base 2268..2269: mod_base = OTHER; note = thymine
  modified_base 2271: mod_base = OTHER; note = thymine
  modified_base 2276: mod_base = OTHER; note = thymine
  modified_base 2279: mod_base = OTHER; note = thymine
  modified_base 2282: mod_base = OTHER; note = thymine
  modified_base 2284: mod_base = OTHER; note = thymine
  modified_base 2286: mod_base = OTHER; note = thymine
  modified_base 2289..2290: mod_base = OTHER; note = thymine
  modified_base 2294: mod_base = OTHER; note = thymine
  modified_base 2297: mod_base = OTHER; note = thymine
  modified_base 2306..2310: mod_base = OTHER; note = thymine
  modified_base 2313: mod_base = OTHER; note = thymine
  modified_base 2315: mod_base = OTHER; note = thymine
  modified_base 2321..2324: mod_base = OTHER; note = thymine
  modified_base 2328: mod_base = OTHER; note = thymine
  modified_base 2330..2334: mod_base = OTHER; note = thymine
  modified_base 2336..2338: mod_base = OTHER; note = thymine
  modified_base 2340: mod_base = OTHER; note = thymine
  modified_base 2349: mod_base = OTHER; note = thymine
  modified_base 2352..2353: mod_base = OTHER; note = thymine
  modified_base 2356: mod_base = OTHER; note = thymine
  modified_base 2361..2364: mod_base = OTHER; note = thymine
  modified_base 2374: mod_base = OTHER; note = thymine
  modified_base 2376: mod_base = OTHER; note = thymine
  modified_base 2386: mod_base = OTHER; note = thymine
  modified_base 2391: mod_base = OTHER; note = thymine
  modified_base 2397: mod_base = OTHER; note = thymine
  modified_base 2399: mod_base = OTHER; note = thymine
  modified_base 2407: mod_base = OTHER; note = thymine
  modified_base 2411..2412: mod_base = OTHER; note = thymine
  modified_base 2414..2415: mod_base = OTHER; note = thymine
  modified_base 2418..2419: mod_base = OTHER; note = thymine
  modified_base 2422..2423: mod_base = OTHER; note = thymine
  modified_base 2425: mod_base = OTHER; note = thymine
  modified_base 2435..2436: mod_base = OTHER; note = thymine
  modified_base 2438: mod_base = OTHER; note = thymine
  modified_base 2440: mod_base = OTHER; note = thymine
  modified_base 2448..2449: mod_base = OTHER; note = thymine
  modified_base 2451: mod_base = OTHER; note = thymine
  modified_base 2455..2457: mod_base = OTHER; note = thymine
  modified_base 2460: mod_base = OTHER; note = thymine
  modified_base 2467..2469: mod_base = OTHER; note = thymine
  modified_base 2471..2472: mod_base = OTHER; note = thymine
  modified_base 2474: mod_base = OTHER; note = thymine
  modified_base 2479: mod_base = OTHER; note = thymine
  modified_base 2481: mod_base = OTHER; note = thymine
  modified_base 2483..2484: mod_base = OTHER; note = thymine
  modified_base 2487: mod_base = OTHER; note = thymine
  modified_base 2491: mod_base = OTHER; note = thymine
  modified_base 2494: mod_base = OTHER; note = thymine
  modified_base 2499..2500: mod_base = OTHER; note = thymine
  modified_base 2502..2504: mod_base = OTHER; note = thymine
  modified_base 2507: mod_base = OTHER; note = thymine
  modified_base 2509..2510: mod_base = OTHER; note = thymine
  modified_base 2517: mod_base = OTHER; note = thymine
  modified_base 2520: mod_base = OTHER; note = thymine
  modified_base 2524: mod_base = OTHER; note = thymine
  modified_base 2527: mod_base = OTHER; note = thymine
  modified_base 2531: mod_base = OTHER; note = thymine
  modified_base 2540..2543: mod_base = OTHER; note = thymine
  modified_base 2545: mod_base = OTHER; note = thymine
  modified_base 2547..2548: mod_base = OTHER; note = thymine
  modified_base 2556: mod_base = OTHER; note = thymine
  modified_base 2559..2560: mod_base = OTHER; note = thymine
  modified_base 2563: mod_base = OTHER; note = thymine
  modified_base 2571..2573: mod_base = OTHER; note = thymine
  modified_base 2576..2577: mod_base = OTHER; note = thymine
  modified_base 2582..2583: mod_base = OTHER; note = thymine
  modified_base 2591..2592: mod_base = OTHER; note = thymine
  modified_base 2597: mod_base = OTHER; note = thymine
  modified_base 2599: mod_base = OTHER; note = thymine
  modified_base 2602: mod_base = OTHER; note = thymine
  modified_base 2606: mod_base = OTHER; note = thymine
  modified_base 2609..2610: mod_base = OTHER; note = thymine
  modified_base 2615: mod_base = OTHER; note = thymine
  modified_base 2617..2618: mod_base = OTHER; note = thymine
  modified_base 2620..2621: mod_base = OTHER; note = thymine
  modified_base 2636: mod_base = OTHER; note = thymine
  modified_base 2638: mod_base = OTHER; note = thymine
  modified_base 2644..2645: mod_base = OTHER; note = thymine
  modified_base 2652: mod_base = OTHER; note = thymine
  modified_base 2657: mod_base = OTHER; note = thymine
  modified_base 2660..2664: mod_base = OTHER; note = thymine
  modified_base 2666: mod_base = OTHER; note = thymine
  modified_base 2668: mod_base = OTHER; note = thymine
  modified_base 2670: mod_base = OTHER; note = thymine
  modified_base 2672..2673: mod_base = OTHER; note = thymine
  modified_base 2677: mod_base = OTHER; note = thymine
  modified_base 2681: mod_base = OTHER; note = thymine
  modified_base 2683: mod_base = OTHER; note = thymine
  modified_base 2692..2693: mod_base = OTHER; note = thymine
  modified_base 2695..2696: mod_base = OTHER; note = thymine
  modified_base 2699..2700: mod_base = OTHER; note = thymine
  modified_base 2704..2705: mod_base = OTHER; note = thymine
  modified_base 2711..2715: mod_base = OTHER; note = thymine
  modified_base 2720..2721: mod_base = OTHER; note = thymine
  modified_base 2723: mod_base = OTHER; note = thymine
  modified_base 2726: mod_base = OTHER; note = thymine
  modified_base 2731..2733: mod_base = OTHER; note = thymine
  modified_base 2736..2737: mod_base = OTHER; note = thymine
  modified_base 2744..2746: mod_base = OTHER; note = thymine
  modified_base 2748..2749: mod_base = OTHER; note = thymine
  modified_base 2756..2758: mod_base = OTHER; note = thymine
  modified_base 2760..2762: mod_base = OTHER; note = thymine
  modified_base 2765..2766: mod_base = OTHER; note = thymine
  modified_base 2776..2777: mod_base = OTHER; note = thymine
  modified_base 2780: mod_base = OTHER; note = thymine
  modified_base 2784: mod_base = OTHER; note = thymine
  modified_base 2788: mod_base = OTHER; note = thymine
  modified_base 2790..2792: mod_base = OTHER; note = thymine
  modified_base 2794: mod_base = OTHER; note = thymine
  modified_base 2796..2797: mod_base = OTHER; note = thymine
  modified_base 2799: mod_base = OTHER; note = thymine
  modified_base 2801: mod_base = OTHER; note = thymine
  modified_base 2806: mod_base = OTHER; note = thymine
  modified_base 2808: mod_base = OTHER; note = thymine
  modified_base 2816: mod_base = OTHER; note = thymine
  modified_base 2825: mod_base = OTHER; note = thymine
  modified_base 2827..2829: mod_base = OTHER; note = thymine
  modified_base 2834: mod_base = OTHER; note = thymine
  modified_base 2838: mod_base = OTHER; note = thymine
  modified_base 2840..2841: mod_base = OTHER; note = thymine
  modified_base 2844: mod_base = OTHER; note = thymine
  modified_base 2846..2847: mod_base = OTHER; note = thymine
  modified_base 2851: mod_base = OTHER; note = thymine
  modified_base 2857: mod_base = OTHER; note = thymine
  modified_base 2861: mod_base = OTHER; note = thymine
  modified_base 2873..2874: mod_base = OTHER; note = thymine
  modified_base 2879: mod_base = OTHER; note = thymine
  modified_base 2882..2884: mod_base = OTHER; note = thymine
  modified_base 2891: mod_base = OTHER; note = thymine
  modified_base 2896: mod_base = OTHER; note = thymine
  modified_base 2898..2899: mod_base = OTHER; note = thymine
  modified_base 2901: mod_base = OTHER; note = thymine
  modified_base 2904..2906: mod_base = OTHER; note = thymine
  modified_base 2910: mod_base = OTHER; note = thymine
  modified_base 2913: mod_base = OTHER; note = thymine
  modified_base 2915..2916: mod_base = OTHER; note = thymine
  modified_base 2920: mod_base = OTHER; note = thymine
  modified_base 2929: mod_base = OTHER; note = thymine
  modified_base 2939..2941: mod_base = OTHER; note = thymine
  modified_base 2946: mod_base = OTHER; note = thymine
  modified_base 2950..2951: mod_base = OTHER; note = thymine
  modified_base 2954: mod_base = OTHER; note = thymine
  modified_base 2956: mod_base = OTHER; note = thymine
  modified_base 2961..2962: mod_base = OTHER; note = thymine
  modified_base 2964: mod_base = OTHER; note = thymine
  modified_base 2966: mod_base = OTHER; note = thymine
  modified_base 2968: mod_base = OTHER; note = thymine
  modified_base 2970..2971: mod_base = OTHER; note = thymine
  modified_base 2973..2975: mod_base = OTHER; note = thymine
  modified_base 2983..2984: mod_base = OTHER; note = thymine
  modified_base 2987: mod_base = OTHER; note = thymine
  modified_base 2989: mod_base = OTHER; note = thymine
  modified_base 2993: mod_base = OTHER; note = thymine
  modified_base 2995..2997: mod_base = OTHER; note = thymine
  modified_base 3002..3003: mod_base = OTHER; note = thymine
  modified_base 3008..3009: mod_base = OTHER; note = thymine
  modified_base 3011: mod_base = OTHER; note = thymine
  modified_base 3013: mod_base = OTHER; note = thymine
  modified_base 3019: mod_base = OTHER; note = thymine
  modified_base 3025: mod_base = OTHER; note = thymine
  modified_base 3032..3034: mod_base = OTHER; note = thymine
  modified_base 3036: mod_base = OTHER; note = thymine
  modified_base 3049..3050: mod_base = OTHER; note = thymine
  modified_base 3053: mod_base = OTHER; note = thymine
  modified_base 3061: mod_base = OTHER; note = thymine
  modified_base 3065: mod_base = OTHER; note = thymine
  modified_base 3070: mod_base = OTHER; note = thymine
  modified_base 3072: mod_base = OTHER; note = thymine
  modified_base 3076..3078: mod_base = OTHER; note = thymine
  modified_base 3082..3083: mod_base = OTHER; note = thymine
  modified_base 3085: mod_base = OTHER; note = thymine
  modified_base 3096: mod_base = OTHER; note = thymine
  modified_base 3108..3112: mod_base = OTHER; note = thymine
  modified_base 3117: mod_base = OTHER; note = thymine
  modified_base 3120: mod_base = OTHER; note = thymine
  modified_base 3124..3126: mod_base = OTHER; note = thymine
  modified_base 3128..3130: mod_base = OTHER; note = thymine
  modified_base 3141: mod_base = OTHER; note = thymine
  modified_base 3146: mod_base = OTHER; note = thymine
  modified_base 3151..3152: mod_base = OTHER; note = thymine
  modified_base 3157..3158: mod_base = OTHER; note = thymine
  modified_base 3175..3176: mod_base = OTHER; note = thymine
  modified_base 3179: mod_base = OTHER; note = thymine
  modified_base 3185: mod_base = OTHER; note = thymine
  modified_base 3189..3190: mod_base = OTHER; note = thymine
  modified_base 3200: mod_base = OTHER; note = thymine
  modified_base 3204: mod_base = OTHER; note = thymine
  modified_base 3210: mod_base = OTHER; note = thymine
  modified_base 3213: mod_base = OTHER; note = thymine
  modified_base 3215: mod_base = OTHER; note = thymine
  modified_base 3220: mod_base = OTHER; note = thymine
  modified_base 3225: mod_base = OTHER; note = thymine
  modified_base 3228: mod_base = OTHER; note = thymine
  modified_base 3230: mod_base = OTHER; note = thymine
  modified_base 3233: mod_base = OTHER; note = thymine
  modified_base 3236: mod_base = OTHER; note = thymine
  modified_base 3240..3241: mod_base = OTHER; note = thymine
  modified_base 3244: mod_base = OTHER; note = thymine
  modified_base 3246: mod_base = OTHER; note = thymine
  modified_base 3248: mod_base = OTHER; note = thymine
  modified_base 3254: mod_base = OTHER; note = thymine
  modified_base 3256: mod_base = OTHER; note = thymine
  modified_base 3259: mod_base = OTHER; note = thymine
  modified_base 3261..3263: mod_base = OTHER; note = thymine
  modified_base 3273: mod_base = OTHER; note = thymine
  modified_base 3282: mod_base = OTHER; note = thymine
  modified_base 3287: mod_base = OTHER; note = thymine
  modified_base 3290..3291: mod_base = OTHER; note = thymine
  modified_base 3294: mod_base = OTHER; note = thymine
  modified_base 3300: mod_base = OTHER; note = thymine
  modified_base 3302: mod_base = OTHER; note = thymine
  modified_base 3306: mod_base = OTHER; note = thymine
  modified_base 3317: mod_base = OTHER; note = thymine
  modified_base 3323: mod_base = OTHER; note = thymine
  modified_base 3332..3333: mod_base = OTHER; note = thymine
  modified_base 3335: mod_base = OTHER; note = thymine
  modified_base 3342..3343: mod_base = OTHER; note = thymine
  modified_base 3347: mod_base = OTHER; note = thymine
  modified_base 3354: mod_base = OTHER; note = thymine
  modified_base 3356: mod_base = OTHER; note = thymine
  modified_base 3360: mod_base = OTHER; note = thymine
  modified_base 3366: mod_base = OTHER; note = thymine
  modified_base 3370: mod_base = OTHER; note = thymine
  modified_base 3372: mod_base = OTHER; note = thymine
  modified_base 3374: mod_base = OTHER; note = thymine
  modified_base 3379: mod_base = OTHER; note = thymine
  modified_base 3385: mod_base = OTHER; note = thymine
  modified_base 3387: mod_base = OTHER; note = thymine
  modified_base 3393..3395: mod_base = OTHER; note = thymine
  modified_base 3401: mod_base = OTHER; note = thymine
  modified_base 3403..3404: mod_base = OTHER; note = thymine
  modified_base 3406: mod_base = OTHER; note = thymine
  modified_base 3408: mod_base = OTHER; note = thymine
  modified_base 3414..3415: mod_base = OTHER; note = thymine
  modified_base 3417: mod_base = OTHER; note = thymine
  modified_base 3419: mod_base = OTHER; note = thymine
  modified_base 3423: mod_base = OTHER; note = thymine
  modified_base 3429..3430: mod_base = OTHER; note = thymine
  modified_base 3432..3434: mod_base = OTHER; note = thymine
  modified_base 3436: mod_base = OTHER; note = thymine
  modified_base 3444: mod_base = OTHER; note = thymine
  modified_base 3451..3454: mod_base = OTHER; note = thymine
  modified_base 3458..3459: mod_base = OTHER; note = thymine
  modified_base 3464..3465: mod_base = OTHER; note = thymine
  modified_base 3470: mod_base = OTHER; note = thymine
  modified_base 3472: mod_base = OTHER; note = thymine
  modified_base 3475..3477: mod_base = OTHER; note = thymine
  modified_base 3480: mod_base = OTHER; note = thymine
  modified_base 3483..3484: mod_base = OTHER; note = thymine
  modified_base 3488..3489: mod_base = OTHER; note = thymine
  modified_base 3509: mod_base = OTHER; note = thymine
  modified_base 3518..3519: mod_base = OTHER; note = thymine
  modified_base 3521: mod_base = OTHER; note = thymine
  modified_base 3525: mod_base = OTHER; note = thymine
  modified_base 3527: mod_base = OTHER; note = thymine
  modified_base 3534..3535: mod_base = OTHER; note = thymine
  modified_base 3543: mod_base = OTHER; note = thymine
  modified_base 3546: mod_base = OTHER; note = thymine
  modified_base 3551: mod_base = OTHER; note = thymine
  modified_base 3554: mod_base = OTHER; note = thymine
  modified_base 3556..3558: mod_base = OTHER; note = thymine
  modified_base 3572: mod_base = OTHER; note = thymine
  modified_base 3575: mod_base = OTHER; note = thymine
  modified_base 3578: mod_base = OTHER; note = thymine
  modified_base 3581: mod_base = OTHER; note = thymine
  modified_base 3586: mod_base = OTHER; note = thymine
  modified_base 3589..3591: mod_base = OTHER; note = thymine
  modified_base 3596..3599: mod_base = OTHER; note = thymine
  modified_base 3601: mod_base = OTHER; note = thymine
  modified_base 3609..3610: mod_base = OTHER; note = thymine
  modified_base 3622: mod_base = OTHER; note = thymine
  modified_base 3630: mod_base = OTHER; note = thymine
  modified_base 3633..3634: mod_base = OTHER; note = thymine
  modified_base 3637: mod_base = OTHER; note = thymine
  modified_base 3643..3645: mod_base = OTHER; note = thymine
  modified_base 3648: mod_base = OTHER; note = thymine
  modified_base 3650: mod_base = OTHER; note = thymine
  modified_base 3653: mod_base = OTHER; note = thymine
  modified_base 3657: mod_base = OTHER; note = thymine
  modified_base 3660: mod_base = OTHER; note = thymine
  modified_base 3662: mod_base = OTHER; note = thymine
  modified_base 3665..3667: mod_base = OTHER; note = thymine
  modified_base 3676..3677: mod_base = OTHER; note = thymine
  modified_base 3686..3687: mod_base = OTHER; note = thymine
  modified_base 3690: mod_base = OTHER; note = thymine
  modified_base 3693: mod_base = OTHER; note = thymine
  modified_base 3696..3697: mod_base = OTHER; note = thymine
  modified_base 3705..3706: mod_base = OTHER; note = thymine
  modified_base 3710..3713: mod_base = OTHER; note = thymine
  modified_base 3718..3719: mod_base = OTHER; note = thymine
  modified_base 3722..3723: mod_base = OTHER; note = thymine
  modified_base 3725: mod_base = OTHER; note = thymine
  modified_base 3728..3729: mod_base = OTHER; note = thymine
  modified_base 3731..3733: mod_base = OTHER; note = thymine
  modified_base 3745: mod_base = OTHER; note = thymine
  modified_base 3756..3757: mod_base = OTHER; note = thymine
  modified_base 3767: mod_base = OTHER; note = thymine
  modified_base 3773: mod_base = OTHER; note = thymine
  modified_base 3776: mod_base = OTHER; note = thymine
  modified_base 3778..3779: mod_base = OTHER; note = thymine
  modified_base 3781: mod_base = OTHER; note = thymine
  modified_base 3783..3784: mod_base = OTHER; note = thymine
  modified_base 3789..3790: mod_base = OTHER; note = thymine
  modified_base 3792: mod_base = OTHER; note = thymine
  modified_base 3798..3802: mod_base = OTHER; note = thymine
  modified_base 3804: mod_base = OTHER; note = thymine
  modified_base 3807: mod_base = OTHER; note = thymine
  modified_base 3812: mod_base = OTHER; note = thymine
  modified_base 3814: mod_base = OTHER; note = thymine
  modified_base 3819: mod_base = OTHER; note = thymine
  modified_base 3821: mod_base = OTHER; note = thymine
  modified_base 3826..3827: mod_base = OTHER; note = thymine
  modified_base 3829: mod_base = OTHER; note = thymine
  modified_base 3831: mod_base = OTHER; note = thymine
  modified_base 3833..3835: mod_base = OTHER; note = thymine
  modified_base 3845: mod_base = OTHER; note = thymine
  modified_base 3848: mod_base = OTHER; note = thymine
  modified_base 3850..3852: mod_base = OTHER; note = thymine
  modified_base 3854: mod_base = OTHER; note = thymine
  modified_base 3859: mod_base = OTHER; note = thymine
  modified_base 3862: mod_base = OTHER; note = thymine
  modified_base 3868..3870: mod_base = OTHER; note = thymine
  modified_base 3876..3877: mod_base = OTHER; note = thymine
  modified_base 3884: mod_base = OTHER; note = thymine
  modified_base 3892: mod_base = OTHER; note = thymine
  modified_base 3895: mod_base = OTHER; note = thymine
  modified_base 3900: mod_base = OTHER; note = thymine
  modified_base 3902: mod_base = OTHER; note = thymine
  modified_base 3904: mod_base = OTHER; note = thymine
  modified_base 3907: mod_base = OTHER; note = thymine
  modified_base 3909..3910: mod_base = OTHER; note = thymine
  modified_base 3913: mod_base = OTHER; note = thymine
  modified_base 3916..3917: mod_base = OTHER; note = thymine
  modified_base 3919..3920: mod_base = OTHER; note = thymine
  modified_base 3922: mod_base = OTHER; note = thymine
  modified_base 3926: mod_base = OTHER; note = thymine
  modified_base 3938..3942: mod_base = OTHER; note = thymine
  modified_base 3954: mod_base = OTHER; note = thymine
  modified_base 3957: mod_base = OTHER; note = thymine
  modified_base 3961..3962: mod_base = OTHER; note = thymine
  modified_base 3964: mod_base = OTHER; note = thymine
  modified_base 3966: mod_base = OTHER; note = thymine
  modified_base 3979..3981: mod_base = OTHER; note = thymine
  modified_base 3984: mod_base = OTHER; note = thymine
  modified_base 3992: mod_base = OTHER; note = thymine
  modified_base 3998..3999: mod_base = OTHER; note = thymine
  modified_base 4002..4004: mod_base = OTHER; note = thymine
  modified_base 4008: mod_base = OTHER; note = thymine
  modified_base 4010: mod_base = OTHER; note = thymine
  modified_base 4014: mod_base = OTHER; note = thymine
  modified_base 4017..4018: mod_base = OTHER; note = thymine
  modified_base 4020: mod_base = OTHER; note = thymine
  modified_base 4025: mod_base = OTHER; note = thymine
  modified_base 4027..4028: mod_base = OTHER; note = thymine
  modified_base 4030..4031: mod_base = OTHER; note = thymine
  modified_base 4036..4038: mod_base = OTHER; note = thymine
  modified_base 4040: mod_base = OTHER; note = thymine
  modified_base 4046: mod_base = OTHER; note = thymine
  modified_base 4051..4053: mod_base = OTHER; note = thymine
  modified_base 4058..4059: mod_base = OTHER; note = thymine
  modified_base 4066..4067: mod_base = OTHER; note = thymine
  modified_base 4071..4073: mod_base = OTHER; note = thymine
  modified_base 4077: mod_base = OTHER; note = thymine
  modified_base 4079: mod_base = OTHER; note = thymine
  modified_base 4083: mod_base = OTHER; note = thymine
  modified_base 4085: mod_base = OTHER; note = thymine
  modified_base 4087..4089: mod_base = OTHER; note = thymine
  modified_base 4093: mod_base = OTHER; note = thymine
  modified_base 4097: mod_base = OTHER; note = thymine
  modified_base 4103..4105: mod_base = OTHER; note = thymine
  modified_base 4109..4110: mod_base = OTHER; note = thymine
  modified_base 4113..4115: mod_base = OTHER; note = thymine
  modified_base 4125: mod_base = OTHER; note = thymine
  modified_base 4128: mod_base = OTHER; note = thymine
  modified_base 4130..4131: mod_base = OTHER; note = thymine
  modified_base 4134: mod_base = OTHER; note = thymine
  modified_base 4148: mod_base = OTHER; note = thymine
  modified_base 4151..4152: mod_base = OTHER; note = thymine
  modified_base 4154: mod_base = OTHER; note = thymine
  modified_base 4166..4168: mod_base = OTHER; note = thymine
  modified_base 4170: mod_base = OTHER; note = thymine
  modified_base 4178..4183: mod_base = OTHER; note = thymine
  modified_base 4188: mod_base = OTHER; note = thymine
  modified_base 4190: mod_base = OTHER; note = thymine
  modified_base 4193: mod_base = OTHER; note = thymine
  modified_base 4205: mod_base = OTHER; note = thymine
  modified_base 4208: mod_base = OTHER; note = thymine
  modified_base 4210..4212: mod_base = OTHER; note = thymine
  modified_base 4215: mod_base = OTHER; note = thymine
  modified_base 4219: mod_base = OTHER; note = thymine
  modified_base 4224: mod_base = OTHER; note = thymine
  modified_base 4229: mod_base = OTHER; note = thymine
  modified_base 4237: mod_base = OTHER; note = thymine
  modified_base 4244: mod_base = OTHER; note = thymine
  modified_base 4246: mod_base = OTHER; note = thymine
  modified_base 4248: mod_base = OTHER; note = thymine
  modified_base 4256: mod_base = OTHER; note = thymine
  modified_base 4265: mod_base = OTHER; note = thymine
  modified_base 4272: mod_base = OTHER; note = thymine
  modified_base 4276..4278: mod_base = OTHER; note = thymine
  modified_base 4281: mod_base = OTHER; note = thymine
  modified_base 4283: mod_base = OTHER; note = thymine
  modified_base 4286: mod_base = OTHER; note = thymine
  modified_base 4289..4290: mod_base = OTHER; note = thymine
  modified_base 4293..4294: mod_base = OTHER; note = thymine
  modified_base 4300: mod_base = OTHER; note = thymine
  modified_base 4303: mod_base = OTHER; note = thymine
  modified_base 4307: mod_base = OTHER; note = thymine
  modified_base 4313: mod_base = OTHER; note = thymine
  modified_base 4315..4316: mod_base = OTHER; note = thymine
  modified_base 4318: mod_base = OTHER; note = thymine
  modified_base 4321: mod_base = OTHER; note = thymine
  modified_base 4325: mod_base = OTHER; note = thymine
  modified_base 4333: mod_base = OTHER; note = thymine
  modified_base 4335..4337: mod_base = OTHER; note = thymine
  modified_base 4339: mod_base = OTHER; note = thymine
  modified_base 4347: mod_base = OTHER; note = thymine
  modified_base 4351..4354: mod_base = OTHER; note = thymine
  modified_base 4356..4357: mod_base = OTHER; note = thymine
  modified_base 4361: mod_base = OTHER; note = thymine
  modified_base 4368: mod_base = OTHER; note = thymine
  modified_base 4370..4371: mod_base = OTHER; note = thymine
  modified_base 4375: mod_base = OTHER; note = thymine
  modified_base 4377..4378: mod_base = OTHER; note = thymine
  modified_base 4383: mod_base = OTHER; note = thymine
  modified_base 4388: mod_base = OTHER; note = thymine
  modified_base 4390: mod_base = OTHER; note = thymine
  modified_base 4394: mod_base = OTHER; note = thymine
  modified_base 4397: mod_base = OTHER; note = thymine
  modified_base 4399: mod_base = OTHER; note = thymine
  modified_base 4402: mod_base = OTHER; note = thymine
  modified_base 4405..4408: mod_base = OTHER; note = thymine
  modified_base 4410: mod_base = OTHER; note = thymine
  modified_base 4412..4414: mod_base = OTHER; note = thymine
  modified_base 4416: mod_base = OTHER; note = thymine
  modified_base 4420..4424: mod_base = OTHER; note = thymine
  modified_base 4426: mod_base = OTHER; note = thymine
  modified_base 4430: mod_base = OTHER; note = thymine
  modified_base 4432: mod_base = OTHER; note = thymine
  modified_base 4435..4437: mod_base = OTHER; note = thymine
  modified_base 4439: mod_base = OTHER; note = thymine
  modified_base 4442..4443: mod_base = OTHER; note = thymine
  modified_base 4449: mod_base = OTHER; note = thymine
  modified_base 4452: mod_base = OTHER; note = thymine
  modified_base 4456: mod_base = OTHER; note = thymine
  modified_base 4458..4459: mod_base = OTHER; note = thymine
  modified_base 4461: mod_base = OTHER; note = thymine
  modified_base 4463: mod_base = OTHER; note = thymine
  modified_base 4468: mod_base = OTHER; note = thymine
  modified_base 4471: mod_base = OTHER; note = thymine
  modified_base 4473..4474: mod_base = OTHER; note = thymine
  modified_base 4476: mod_base = OTHER; note = thymine
  modified_base 4479: mod_base = OTHER; note = thymine
  modified_base 4481..4482: mod_base = OTHER; note = thymine
  modified_base 4487..4490: mod_base = OTHER; note = thymine
  modified_base 4492..4493: mod_base = OTHER; note = thymine
  modified_base 4500..4503: mod_base = OTHER; note = thymine
  modified_base 4512..4513: mod_base = OTHER; note = thymine
  modified_base 4515: mod_base = OTHER; note = thymine
  modified_base 4517: mod_base = OTHER; note = thymine
  modified_base 4519..4520: mod_base = OTHER; note = thymine
  modified_base 4522..4523: mod_base = OTHER; note = thymine
  modified_base 4525: mod_base = OTHER; note = thymine
  modified_base 4529: mod_base = OTHER; note = thymine
  modified_base 4537: mod_base = OTHER; note = thymine
  modified_base 4539..4541: mod_base = OTHER; note = thymine
  modified_base 4543: mod_base = OTHER; note = thymine
  modified_base 4550..4551: mod_base = OTHER; note = thymine
  modified_base 4553: mod_base = OTHER; note = thymine
  modified_base 4555..4556: mod_base = OTHER; note = thymine
  modified_base 4561: mod_base = OTHER; note = thymine
  modified_base 4563: mod_base = OTHER; note = thymine
  modified_base 4567: mod_base = OTHER; note = thymine
  modified_base 4569: mod_base = OTHER; note = thymine
  modified_base 4571: mod_base = OTHER; note = thymine
  modified_base 4573..4574: mod_base = OTHER; note = thymine
  modified_base 4576: mod_base = OTHER; note = thymine
  modified_base 4580..4581: mod_base = OTHER; note = thymine
  modified_base 4590: mod_base = OTHER; note = thymine
  modified_base 4597: mod_base = OTHER; note = thymine
  modified_base 4599: mod_base = OTHER; note = thymine
  modified_base 4606: mod_base = OTHER; note = thymine
  modified_base 4610..4611: mod_base = OTHER; note = thymine
  modified_base 4618..4620: mod_base = OTHER; note = thymine
  modified_base 4622: mod_base = OTHER; note = thymine
  modified_base 4624..4625: mod_base = OTHER; note = thymine
  modified_base 4628: mod_base = OTHER; note = thymine
  modified_base 4631: mod_base = OTHER; note = thymine
  modified_base 4633: mod_base = OTHER; note = thymine
  modified_base 4637: mod_base = OTHER; note = thymine
  modified_base 4641: mod_base = OTHER; note = thymine
  modified_base 4646..4647: mod_base = OTHER; note = thymine
  modified_base 4649: mod_base = OTHER; note = thymine
  modified_base 4652..4655: mod_base = OTHER; note = thymine
  modified_base 4657..4660: mod_base = OTHER; note = thymine
  modified_base 4662..4663: mod_base = OTHER; note = thymine
  modified_base 4671: mod_base = OTHER; note = thymine
  modified_base 4673: mod_base = OTHER; note = thymine
  modified_base 4677: mod_base = OTHER; note = thymine
  modified_base 4680..4682: mod_base = OTHER; note = thymine
  modified_base 4686: mod_base = OTHER; note = thymine
  modified_base 4695: mod_base = OTHER; note = thymine
  modified_base 4705..4707: mod_base = OTHER; note = thymine
  modified_base 4709..4710: mod_base = OTHER; note = thymine
  modified_base 4715: mod_base = OTHER; note = thymine
  modified_base 4718: mod_base = OTHER; note = thymine
  modified_base 4720..4721: mod_base = OTHER; note = thymine
  modified_base 4729: mod_base = OTHER; note = thymine
  modified_base 4732..4733: mod_base = OTHER; note = thymine
  modified_base 4735: mod_base = OTHER; note = thymine
  modified_base 4739..4741: mod_base = OTHER; note = thymine
  modified_base 4745: mod_base = OTHER; note = thymine
  modified_base 4748..4749: mod_base = OTHER; note = thymine
  modified_base 4751..4752: mod_base = OTHER; note = thymine
  modified_base 4764..4767: mod_base = OTHER; note = thymine
  modified_base 4769..4770: mod_base = OTHER; note = thymine
  modified_base 4773: mod_base = OTHER; note = thymine
  modified_base 4781: mod_base = OTHER; note = thymine
  modified_base 4783: mod_base = OTHER; note = thymine
  modified_base 4786: mod_base = OTHER; note = thymine
  modified_base 4789: mod_base = OTHER; note = thymine
  modified_base 4791: mod_base = OTHER; note = thymine
  modified_base 4798: mod_base = OTHER; note = thymine
  modified_base 4805..4806: mod_base = OTHER; note = thymine
  modified_base 4809: mod_base = OTHER; note = thymine
  modified_base 4812: mod_base = OTHER; note = thymine
  modified_base 4817: mod_base = OTHER; note = thymine
  modified_base 4819: mod_base = OTHER; note = thymine
  modified_base 4824: mod_base = OTHER; note = thymine
  modified_base 4826..4828: mod_base = OTHER; note = thymine
  modified_base 4832..4833: mod_base = OTHER; note = thymine
  modified_base 4835..4836: mod_base = OTHER; note = thymine
  modified_base 4838: mod_base = OTHER; note = thymine
  modified_base 4840: mod_base = OTHER; note = thymine
  modified_base 4843..4845: mod_base = OTHER; note = thymine
  modified_base 4851..4852: mod_base = OTHER; note = thymine
  modified_base 4857: mod_base = OTHER; note = thymine
  modified_base 4862..4863: mod_base = OTHER; note = thymine
  modified_base 4865..4866: mod_base = OTHER; note = thymine
  modified_base 4870: mod_base = OTHER; note = thymine
  modified_base 4881..4882: mod_base = OTHER; note = thymine
  modified_base 4887: mod_base = OTHER; note = thymine
  modified_base 4894..4895: mod_base = OTHER; note = thymine
  modified_base 4901..4902: mod_base = OTHER; note = thymine
  modified_base 4909: mod_base = OTHER; note = thymine
  modified_base 4914..4915: mod_base = OTHER; note = thymine
  modified_base 4917: mod_base = OTHER; note = thymine
  modified_base 4919: mod_base = OTHER; note = thymine
  modified_base 4922: mod_base = OTHER; note = thymine
  modified_base 4928: mod_base = OTHER; note = thymine
  modified_base 4932..4934: mod_base = OTHER; note = thymine
  modified_base 4936..4938: mod_base = OTHER; note = thymine
  modified_base 4941: mod_base = OTHER; note = thymine
  modified_base 4944: mod_base = OTHER; note = thymine
  modified_base 4948: mod_base = OTHER; note = thymine
  modified_base 4950: mod_base = OTHER; note = thymine
  modified_base 4952..4953: mod_base = OTHER; note = thymine
  modified_base 4956..4958: mod_base = OTHER; note = thymine
  modified_base 4961: mod_base = OTHER; note = thymine
  modified_base 4964: mod_base = OTHER; note = thymine
  modified_base 4975..4976: mod_base = OTHER; note = thymine
  modified_base 4978..4979: mod_base = OTHER; note = thymine
  modified_base 4982: mod_base = OTHER; note = thymine
  modified_base 4989: mod_base = OTHER; note = thymine
  modified_base 4992..4993: mod_base = OTHER; note = thymine
  modified_base 4996..4997: mod_base = OTHER; note = thymine
  modified_base 5000..5001: mod_base = OTHER; note = thymine
  modified_base 5006..5007: mod_base = OTHER; note = thymine
  modified_base 5023..5024: mod_base = OTHER; note = thymine
  modified_base 5028..5031: mod_base = OTHER; note = thymine
  modified_base 5036: mod_base = OTHER; note = thymine
  modified_base 5045..5048: mod_base = OTHER; note = thymine
  modified_base 5056..5059: mod_base = OTHER; note = thymine
  modified_base 5061: mod_base = OTHER; note = thymine
  modified_base 5063: mod_base = OTHER; note = thymine
  modified_base 5066: mod_base = OTHER; note = thymine
  modified_base 5068: mod_base = OTHER; note = thymine
  modified_base 5070..5071: mod_base = OTHER; note = thymine
  modified_base 5076: mod_base = OTHER; note = thymine
  modified_base 5078: mod_base = OTHER; note = thymine
  modified_base 5085: mod_base = OTHER; note = thymine
  modified_base 5087: mod_base = OTHER; note = thymine
  modified_base 5090..5092: mod_base = OTHER; note = thymine
  modified_base 5094: mod_base = OTHER; note = thymine
  modified_base 5097: mod_base = OTHER; note = thymine
  modified_base 5102: mod_base = OTHER; note = thymine
  modified_base 5111: mod_base = OTHER; note = thymine
  modified_base 5113: mod_base = OTHER; note = thymine
  modified_base 5120..5121: mod_base = OTHER; note = thymine
  modified_base 5127: mod_base = OTHER; note = thymine
  modified_base 5131..5133: mod_base = OTHER; note = thymine
  modified_base 5137..5140: mod_base = OTHER; note = thymine
  modified_base 5145: mod_base = OTHER; note = thymine
  modified_base 5156..5157: mod_base = OTHER; note = thymine
  modified_base 5159: mod_base = OTHER; note = thymine
  modified_base 5166: mod_base = OTHER; note = thymine
  modified_base 5175: mod_base = OTHER; note = thymine
  modified_base 5178..5182: mod_base = OTHER; note = thymine
  modified_base 5184..5185: mod_base = OTHER; note = thymine
  modified_base 5191: mod_base = OTHER; note = thymine
  modified_base 5193: mod_base = OTHER; note = thymine
  modified_base 5201..5202: mod_base = OTHER; note = thymine
  modified_base 5204: mod_base = OTHER; note = thymine
gcgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcacccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcgatgtcctcggccaccgacccgcccagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatatagccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattgtgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgaaaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagatgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattcgactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattgtgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

(19) United States
(12) Patent Application Publication
(10) Pub. No.: US 2024/0252611 A1
(43) Pub. Date: Aug. 1, 2024

(54) ARENAVIRUSES FOR USE IN THE TREATMENT AND/OR PREVENTION OF TUMORS AND METHOD FOR PRODUCING ARENAVIRUSES WITH (IM-PROVED) TUMOR-REGRESSION PROPERTIES

(71) Applicant: ABALOS THERAPEUTICS GMBH, Duesseldorf (DE)

(72) Inventors: Karl Sebastian Lang, Essen (DE); Halime Kalkavan, Memphis, TN (US)

(73) Assignee: ABALOS THERAPEUTICS GMBH, Duesseldorf (DE)

(21) Appl. No.: 18/385,250

(22) Filed: Oct. 30, 2023

Related U.S. Application Data
(63) Continuation of application No. 16/267,095, filed on Feb. 4, 2019, now Pat. No. 11,801,294.
Application No. 16/267,095 is a division of application No. 15/567,343, filed on Oct. 17, 2017, now abandoned.

(30) Foreign Application Priority Data
Apr. 17, 2015 (DE) 102015207036.0

(51) Int. Cl. A61K 39/12; A61K 35/76; A61K 35/768; A61K 39/00; A61P 35/00; C12N 7/00

(52) U.S. Cl. CPC A61K 39/12; A61K 35/76; A61K 35/768; C12N 7/00; A61K 2039/5254; A61K 2039/585; A61P 35/00; C12N 2760/10011; C12N 2760/10021; C12N 2760/10032; C12N 2760/10033

(57) ABSTRACT

The invention relates to arenaviruses for use in the treatment and/or prevention of tumors and also a method for preparing arenaviruses with (improved) tumor-regressive properties.

### CROSS-REFERENCE TO RELATED APPLICATIONS

The present application is a continuation of U.S. patent application Ser. No. 16/267,095, filed Feb. 4, 2019, now U.S. Pat. No. 11,801,294, which is a divisional application of U.S. patent application Ser. No. 15/567,343, filed Oct. 17, 2017, which is the United States National stage patent application of International Application No. PCT/EP2016/058347, filed Apr. 15, 2016, which claims the right of priority of German patent application No. 102015207036.0 filed Apr. 17, 2015, with the German Patent Office, the entire contents of each of which are incorporated herein for all purposes.

### SEQUENCE LISTING

The application contains a Sequence Listing which has been submitted electronically in .XML format and is hereby incorporated by reference in its entirety. Said .XML copy, created on Mar. 27, 2024, is named “SCH-5000-CT1_SL.xml” and is 2,758, 175 bytes in size. The sequence listing contained in this .XML file is part of the specification and is hereby incorporated by reference herein in its entirety.

### FIELD OF THE INVENTION

The invention relates to arenaviruses for use in the treatment and/or prevention of tumors and also methods for preparing arenaviruses with (improved) tumor-regressive properties.

Arenaviruses belong to the family of human pathogenic, pleomorphic RNA viruses. Diseases with these viruses belong to the zoonoses due to their natural reservoir in animals, predominantly rodents. Zoonoses refer to diseases that can be transferred from the animal to humans and vice versa from humans to the animal.

At least eight arenaviruses are known to cause illness in humans. Typical are aseptic meningitis and haemorrhagic fever. Known viruses which can trigger a disease in humans are the lymphocytic choriomeningitis virus (LCMV), Guanarito virus (GTOV), Junin virus (JUNV), Lassa virus (LASV), Lujo virus (LUJV) Machupo virus (MACV), Sabia virus (SABV) and the Whitewater Arroyo virus (WWAV).

Arenaviruses are generally divided into two groups, namely the Old World arenaviruses and the New World arenaviruses. These groups differ geographically and genetically. Old World arenaviruses, such as the lymphocytic choriomeningitis virus, have been found in countries of the eastern hemisphere, such as European, Asian and African countries. In contrast, New World arenaviruses have been found in countries of the western hemisphere, such as Argentina, Bolivia, Venezuela, Brazil and the United States of America.

The name of the virus family is derived from the Latin arenosus (sandy) and arena (sand) to describe the sandy ribosomal structure within the virions. The virions of the arenaviruses have a round to irregular shape and have a diameter, depending on species and preparation of the test material, from 50 nm to 300 nm, usually between 110 nm and 130 nm. Club-shaped glycoprotein spikes, 8 nm to 10 nm long, are embedded in the virus envelope. The individual spikes consist of a tetramer of the viral envelope protein.

The virions also comprise two closed-ring capsids with helical symmetry. The length of the capsids varies between 450 nm and 1300 nm. One molecule of the viral RNA (ribonucleic acid) polymerase (L-protein) is attached to each of them.

Each capsid comprises one molecule of a single-stranded RNA with mixed (i.e. ambisense, +/−) polarity. The two single-stranded RNA molecules represent the viral genome. They are referred to as L (large) and S (small) and are about 7.5 kb (kilobases) or 3.5 kb (kilobases) large. Although the capsids are closed ring-shaped, the RNA strands are linear and thus not circular. A 19 to 30 base long sequence at the 3′ end of the RNA is present on both strands and is also conserved within the virus family.

Very exceptional morphologically is the presence of an alternating number of cellular ribosomes within the virions, which give the viral particles their “sandy” appearance. Similarly, in purified virus preparations, a number of different cellular RNAs (also including ribosomal RNA) and also replicative forms of viral RNA are found, as are diverse viral mRNAs (messenger ribonucleic acids bound to the ribosomes) and complete complementary strands of the virus genome. These non-genomic RNAs are found in varying amounts all lying outside the abovementioned capsids.

The use of arenaviruses as vaccination vectors is known. A prominent example is the vaccination virus Candid #1 used against Argentinian hemorrhagic fever. This is a vaccination variant of the Junin virus.

Known from WO 2009/083210 A1 is the use of replication defects, i.e. genetically modified arenavirus particles (virions), inter alia, for the treatment of neo-plastic diseases such as, for example, melanoma, prostate carcinoma, breast carcinoma and lung carcinoma. In the publication “Development of replication-defective lymphocytic choriomeningitis virus vectors for the induction of potent CD8⁺ T cell immunity” (Nature Medicine, Vol. 16, No. 3, Mar. 2010, pp. 339-345; doi: 10.1038/Nm.2104), cancer immunotherapy is mentioned as a potential area of application for such viral particles.

Furthermore, WO 2006/008074 A1 discloses the use of packaging cells, which produce retroviral virions pseudotyped with arenavirus glycoprotein, for gene therapy of solid tumors.

The methods for the treatment of tumors described in the prior art are based on the use of virus particles which are very complicated to produce by genetic engineering. In the case of gene therapy treatment methods, it is frequently not possible to achieve adequate, therapeutically effective transduction of the tumor tissue with genetically engineered virions or packaging cells which produce virions.

### SUMMARY OF THE INVENTION

### Object and Solution

The present invention is therefore based on the object of providing a simpler and, in particular, more efficient therapeutic solution for tumors, in particular carcinomas and sarcomas, compared to the prior art.

This object is achieved according to the invention by an arenavirus according to independent claim 1, by a medicament according to claim 14 and also by an in vitro method according to independent claim 15. Preferred embodiments are defined in the dependent claims. The wording of all claims is hereby incorporated by reference into the content of the description. An additional subject of the invention, which achieves the object of the invention, is disclosed in the description.

According to a first aspect, the invention relates to an arenavirus for use in the treatment and/or prevention of a tumor, preferably a malignant tumor, in humans or animals.

The arenavirus is preferably characterized in that it is free of genomic foreign RNA, i.e. it does not comprise any genomic foreign RNA. In other words, the genome of the arenavirus is preferably free of foreign RNA or preferably comprises no foreign RNA.

In the context of the present invention, the expression “genomic foreign RNA” is intended to mean an RNA (ribonucleic acid) or RNA sequence which does not occur or is not present in the genome of a wild-type arenavirus or in the genome of a mutant of a wild-type arenavirus (mutated arenavirus), in particular in the genome of a natural mutant of a wild-type arenavirus (naturally mutated arenavirus). Examples of foreign RNA are artificial or synthetic RNA molecules, RNA of organisms and RNA from other viruses.

In the context of the present invention, (in accordance with the understanding of those skilled in the art), the expression “wild-type arenavirus” is understood to mean an arenavirus whose genome is the genetically normal form occurring in nature.

In the context of the present invention, (in accordance with the understanding of those skilled in the art), the expression “mutant of a wild-type arenavirus” or “mutated arenavirus” is understood to mean an arenavirus whose genome comprises a spontaneous, i.e. naturally-induced, modification or modification induced by mutagenesis, compared to the wild-type genome.

Accordingly, in the context of the present invention, (in accordance with the understanding of those skilled in the art), the expression “natural mutant of a wild-type arenavirus” or “naturally mutated arenavirus” is understood to mean an arenavirus whose genome comprises a spontaneous, i.e. naturally-induced, modification, compared to the wild-type genome. A naturally mutated arenavirus can be produced preferably by passage, in particular serial passage, which will be discussed in more detail below.

The invention is based on the surprising finding that arenaviruses without genomic foreign RNA are able to effect tumor regression. Tumor regression is due to an activation or stimulation of congenital and adaptive immune cells caused by the arenaviruses. The activated immune cells secrete increased antitumoral cytokines such as interferon-α and interferon-γ, thereby counteracting or repelling the tumor. A further surprising finding is the realization that the arenaviruses cause a significantly increased secretion of antitumoral cytokines in the case of a tumor manifestation. Arenaviruses without genomic foreign RNA are thus suitable for use in tumor treatment. This has been successfully verified by the applicant by means of animal experiments. For this purpose, mice were used, inter alia, in which growth of human tumors is possible.

In a further embodiment, the arenavirus is also free of non-genomic foreign RNA. In the context of the present invention, the expression “non-genomic foreign RNA” is intended to mean an RNA or RNA sequence, apart from the arenavirus genome, which does not occur or is not present in a wild-type arenavirus or a mutant of a wild-type arenavirus (mutated arenavirus), in particular of a natural mutant of a wild-type arenavirus (naturally mutated arenavirus). In other words, in this embodiment the arenavirus does not comprise overall any foreign RNA, i.e neither genomic foreign RNA nor non-genomic foreign RNA.

In a preferred embodiment, the arenavirus is a wild-type arenavirus.

In a further embodiment, the arenavirus is a natural mutant of a wild-type arenavirus, i.e. a naturally mutated arenavirus.

The natural mutant or the naturally mutated arenavirus is preferably produced by passage, in particular multiple passage, in host animals and/or host cells.

The natural mutant or the naturally mutated arenavirus is particularly preferably produced by serial passage in host animals and/or host cells.

The arenavirus provided according to the invention is thus preferably an arenavirus which is produced starting from its wild-type form by passage, preferably serial passage, in host animals and/or host cells.

The host animals mentioned in the preceding paragraphs are preferably rodents, particularly mice. The host cells mentioned in the previous paragraphs, on the other hand, are preferably dendritic cells or tumor cells.

In the context of the present invention, in accordance with the understanding of those skilled in the art, the term “passage” is understood to mean a multiple, regular introduction of the arenavirus into host animals and/or host cells. In the context of the present invention, in accordance with the understanding of those skilled in the art, the expression “serial passage” is understood to mean a multiple, regular introduction of the arenavirus into different host animals, preferably of the same type, and/or different cells, preferably of the same type. Due to the multiple changes of environment (host animal and/or host cell), the arenavirus is subject to an increased adaptation pressure or mutational pressure, thereby increasing the likelihood of advantageous mutations occurring in the genome of the arenavirus from the perspective of tumor regression.

In a further embodiment, the tumor is selected from the group comprising or consisting of carcinoma, melanoma, blastoma, lymphoma and sarcoma.

In the context of the present invention, (in accordance with the understanding of those skilled in the art), the term “carcinoma” is intended to mean malignant neoplasia of epithelial origin.

In the context of the present invention, (in accordance with the understanding of those skilled in the art), the term “sarcoma” is intended to mean malignant neoplasia of mesodermal origin.

In the context of the present invention, (in accordance with the understanding of those skilled in the art), the term “melanoma” is intended to mean malignant neoplasia of melanocytic origin.

In the context of the present invention, (in accordance with the understanding of those skilled in the art), the term “lymphoma” is intended to mean malignant neoplasia of lymphocytic origin.

In the context of the present invention, (in accordance with the understanding of those skilled in the art), the term “blastoma” is intended to mean malignant neoplasia of embryonic origin.

In a preferred embodiment, the carcinoma is selected from the group comprising or consisting of anal carcinoma, bronchial carcinoma, lung carcinoma, endometrial carcinoma, gallbladder carcinoma, hepatocellular carcinoma, testicular carcinoma, colorectal carcinoma, laryngeal carcinoma, oesophogeal cancer, gastric carcinoma, breast carcinoma, renal carcinoma, ovarian carcinoma, pancreas tumor, pharyngeal carcinoma, prostate carcinoma, thyroid carcinoma and cervical carcinoma.

In a preferred embodiment, the sarcoma is selected from the group comprising or consisting of angiosarcoma, chondrosarcoma, Ewing's sarcoma, fibrosarcoma, Kaposi's sarcoma, liposarcoma, leiomyosarcoma, malignant fibrous histiocytoma, neurogenic sarcoma, osteosarcoma and rhabdomyosarcoma.

In a preferred embodiment, the arenavirus is an Old World arenavirus which is preferably selected from the group comprising or consisting of Ippy virus (IP-PYV), Lassa virus (LASV), lymphocytic choriomeningitis virus (LCMV), Mobala virus (MOBV) and Mopeia virus (MOPV).

In a particularly preferred embodiment, the arenavirus is the lymphocytic choriomeningitis virus, preferably a strain which is selected from the group comprising or consisting of WE, Armstrong, Clone 13 and Docile.

In a preferred embodiment, the arenavirus is a New World arenavirus, which is preferably selected from the group comprising or consisting of Allpaahuayo virus (ALLV), Amapari virus (AMAV), Bear Canyon virus (BCNV), Chapare virus, Cupixi virus (CPXV), Flexal virus (FLEV), Guanarito virus (GTOV), Junin virus (JUNV), Latino virus (LATV), Machupo virus (MACV), Oliveros virus (OLVV), Parana virus (PARV), Pichinde virus (PICV), Pirital virus (PIRV), Sabia virus (SABV), Tacaribe virus (TCRV), Tamiami virus (TAMV) and Whitewara Arroyo virus (WWAV).

In a particularly preferred embodiment, the arenavirus is a Junin virus, in particular the strain Candid #1 (Candid No.1).

In a further embodiment, the Junin virus, in particular the strain Candid #1 (Candid No.1), has a nucleic acid sequence, in particular an S-ribonucleic acid sequence or ambisense sequence, according to SEQ ID No. 1 (according to sequence listing).

In a further embodiment, the Junin virus, in particular the strain Candid #1 (Candid No.1), has a nucleic acid sequence, in particular an L-ribonucleic acid sequence or ambisense sequence, according to SEQ ID No. 2 (according to sequence listing).

In a further embodiment, the Junin virus, in particular the strain Candid #1 (Candid No.1), has a nucleic acid sequence, in particular an S-ribonucleic acid sequence or ambisense sequence, according to SEQ ID No. 3 (according to sequence listing).

In a further embodiment, the Junin virus, in particular the strain Candid #1 (Candid No.1), has a nucleic acid sequence, in particular an L-ribonucleic acid sequence or ambisense sequence, according to SEQ ID No. 4 (according to sequence listing).

In a further embodiment, the lymphocytic choriomeningitis virus (LCMV virus) mentioned above has a nucleic acid sequence, in particular an S-ribonucleic acid sequence or ambisense sequence, according to SEQ ID No. 5 (according to sequence listing).

In a further embodiment, the lymphocytic choriomeningitis virus (LCMV virus) mentioned above has a nucleic acid sequence, in particular an L-ribonucleic acid sequence or ambisense sequence, according to SEQ ID No. 6 (according to sequence listing).

In a further embodiment, the arenavirus is isolated from tumor lysates, or -gan lysates, urine or blood.

In an alternative embodiment, the arenavirus is isolated from a cell culture medium, in particular from a human tumor cell line.

In a further embodiment, the arenavirus is for administration in the form of virions, i.e. in the form of arenavirus particles, which are outside a cell.

In a further embodiment, the arenavirus is provided, preferably prepared, for local, in particular intramuscular, intraperitoneal or subcutaneous administration. In other words, the arenavirus is used in a further embodiment for local, in particular intramuscular, intraperitoneal or subcutaneous administration.

Preferably the arenavirus is provided, preferably prepared, for local administration at a dose of 1 PFU (Plaque Forming Unit)/kg body weight to 10¹² PFU/kg body weight, particularly 10² PFU/kg body weight to 10⁶ PFU/kg body weight, preferably 10³ PFU/kg body weight to 10⁵ PFU/kg body weight. In other words, the arenavirus is used, preferably for local administration, at a dose of 1 PFU (Plaque Forming Unit)/kg body weight to 10¹² PFU/kg body weight, particularly 10² PFU/kg body weight to 10⁶ PFU/kg body weight, preferably 10³ PFU/kg body weight to 10⁵ PFU/kg body weight.

In an alternative embodiment, the arenavirus is provided, preferably prepared, for systemic, in particular intravenous, administration. In other words, the arenavirus is used in an alternative embodiment for systemic, in particular intravenous, administration.

Preferably, the arenavirus is provided, preferably prepared, for systemic administration at a dose of 1 PFU/kg body weight to 10¹² PFU/kg body weight, particularly 10² PFU/kg body weight to 10⁶ PFU/kg body weight, preferably 10³ PFU/kg body weight to 10⁵ PFU/kg body weight. In other words, the arenavirus is used, preferably for systemic administration, at a dose of 1 PFU/kg body weight to 10¹² PFU/kg body weight, particularly 10² PFU/kg body weight to 10⁶ PFU/kg body weight, preferably 10³ PFU/kg body weight to 10⁵ PFU/kg body weight.

According to a second aspect, the invention relates to a medicament for use in the treatment and/or prevention of a tumor, in particular a malignant tumor.

The medicament is characterized in particular by the fact that it has an arenavirus according to the first aspect of the invention.

The medicament preferably further comprises a pharmaceutically acceptable carrier. The carrier may in particular be selected from the group comprising or consisting of water, saline solution, buffer solution and cell culture medium.

In a further embodiment, the medicament also comprises an active ingredient. The active ingredient can in particular be a cytostatic agent, an antibody and/or a cytokine.

With regard to further features and advantages of the medicament, in particular of the arenavirus and also the tumor, reference is made fully to the present description in order to avoid repetitions.

According to a third aspect, the invention relates to a method for producing an arenavirus with tumor-regressive, i.e. tumor-repelling/counteracting properties or improved tumor-regressive properties.

The tumor is preferably a malignant tumor, preferably a carcinoma, melanoma, blastoma, lymphoma or sarcoma. Accordingly, the method is preferably a method for producing an arenavirus with (improved) carcinoma-, melanoma-, blastoma-, lymphoma- or sarcoma-regressive properties.

The method comprises the following steps:
a) infecting dendritic cells or tumor cells with an arenavirus,
b) culturing the arenavirus in the infected dendritic cells or infected tumor cells and
c) isolating the cultured arenavirus or a subset of the cultured arenavirus from the infected dendritic cells or infected tumor cells.

The sequence of steps a) to c) may also be referred to as a (single) passage of the arenavirus in the dendritic cells or tumor cells.

In a preferred embodiment, the arenavirus is an arenavirus which has been subjected to a serial passage in host animals prior to carrying out step a). For further details and advantages, reference may be made to the corresponding statements made in the context of the fourth aspect of the invention.

In a further embodiment, the dendritic cells or tumor cells are in the form of a cell culture (dendritic cell culture or tumor cell culture).

The tumor cells are preferably malignant tumor cells, in particular carcinoma, melanoma, blastoma, lymphoma or sarcoma cells.

The carcinoma cells may be selected from the group comprising or consisting of anal carcinoma cells, bronchial carcinoma cells, lung carcinoma cells, endometrial carcinoma cells, gallbladder carcinoma cells, hepatocellular carcinoma cells, testicular carcinoma cells, colorectal carcinoma cells, laryngeal carcinoma cells, oesophogeal carcinoma cells, gastric carcinoma cells, breast carcinoma cells, renal carcinoma cells, ovarian carcinoma cells, pancreas tumor cells, pharyngeal carcinoma cells, prostate carcinoma cells, thyroid carcinoma cells and cervical carcinoma cells.

The sarcoma cells may be selected from the group comprising or consisting of angiosarcoma cells, chondrosarcoma cells, Ewing's sarcoma cells, fibrosarcoma cells, Kaposi's sarcoma cells, liposarcoma cells, leiomyosarcoma cells, malignant fibrous histiocytoma cells, neurogenic sarcoma cells, osteosarcoma cells and rhabdo-myosarcoma cells.

In a further embodiment, the tumor cells are immortalized immune cells, in particular immortalized macrophages.

The dendritic cells or tumor cells are infected according to step a) preferably by adding the arenavirus to the cells.

In a preferred embodiment, the sequence of steps a) to c) is repeated with new, in particular non-infected, dendritic cells, preferably of the same type, or with new, in particular non-infected, tumor cells, preferably of the same type (the same tumor type).

In a particularly preferred embodiment, the sequence of steps a) to c) is repeated many times. By way of preference, new, in particular non-infected, dendritic cells, preferably of the same type, or new, in particular non-infected, tumor cells, preferably of the same type, are used for each repetition.

The sequence of steps a) to c) is preferably repeated once to 1000 times, particularly 10 times to 100 times, preferably 30 times to 60 times, wherein new, in particular non-infected, dendritic cells, preferably of the same type, or new, in particular non-infected, tumor cells, preferably of the same type, are used for each repetition.

By multiple repetition of steps a) to c), the arenavirus is constantly forced to adapt to a new environment, i.e. to new dendritic cells or tumor cells. This permanent adaptation pressure favors the occurrence of mutations, which can produce or improve the tumor-regressive properties of the arenavirus.

During the culturing of the arenavirus according to step b), a replication of the arenavirus genome and a propagation of the arenavirus occur within the dendritic cells or tumor cells.

The arenavirus according to step b) is preferably cultured under standard cell culture conditions.

The arenavirus is preferably cultured in the dendritic cells or tumor cells for a period of 1 minute to 1 year, in particular 10 hours to 1 month, preferably 24 hours to 72 hours.

The cultured arenavirus according to step c) is preferably isolated from a cell culture supernatant.

Preferably, the dendritic cells or tumor cells are sorted according to specific properties, preferably by means of a cell sorting device, and subsequently cultured, prior to isolating according to step c). The sorted cells are preferably cultured over a period of 24 hours.

In a preferred embodiment, the method further comprises the following steps:
d) cloning the isolated arenavirus and
e) sequenzing the isolated arenavirus.

With regard to further features and advantages of the method, in particular of the arenavirus and also the tumor, reference is made to the present description.

According to a fourth aspect, the invention relates to a method for producing an arenavirus with tumor-regressive, i.e. tumor-repelling/counteracting, properties or improved tumor-regressive properties.

The tumor is preferably a malignant tumor, preferably a carcinoma, melanoma, blastoma, lymphoma or sarcoma. Accordingly, the method is preferably a method for producing an arenavirus with (improved) carcinoma-, melanoma-, blastoma-, lymphoma- or sarcoma-regressive properties.

The method comprises the following steps:
a) infecting a host animal, which has a tumor, with an arenavirus,
b) culturing the arenavirus in the infected host animal and
c) isolating the cultured arenavirus or a subset of the cultured arenavirus from the infected host animal.

The sequence of steps a) to c) may also be referred to as a (single) passage of the arenavirus in the host animal.

In a preferred embodiment, tumor tissue is transplanted to the host animal prior to carrying out step a).

In an alternative embodiment, a genetically modified host animal is used which spontaneously develops the tumor.

Rodents in particular, preferably mice, can be used as host animals. For example, NOD SCID mice or LoxP-Tag mice can be used as host animals. In the Nod SCID mice, the SCID (Severe Combined Immunodeficiency) mutation is combined with a NOD (non-obese diabetic) type. In mice that are homozygous for the SCID mutation, no functional T-cells or B-cells are formed. By means of this immunodeficiency, these animals are exceptionally suitable for tolerating foreign body cells, for example transplanted tumors.

The host animal can be infected according to step a), for example by systemic, particularly intravenous, or local, for example subcutaneous, administration of the arenavirus.

In particular, the host animal can be infected by administration, preferably injection, of the arenavirus into the tumor of the host animal.

In a preferred embodiment, the sequence of steps a) to c) is repeated with a new, in particular non-infected, host animal, preferably of the same type.

In a particularly preferred embodiment, the sequence of steps a) to c) is repeated many times. A new, in particular non-infected host animal, preferably of the same type, is preferably used for each repetition. In other words, it is preferred according to the invention if the arenavirus is subjected to serial passage in host animals, preferably of the same type.

The sequence of steps a) to c) is preferably repeated once to 1000 times, particularly 10 times to 100 times, preferably 30 times to 60 times, wherein a new, in particular non-infected, host animal, preferably of the same type, is used for each repetition.

By multiple repetition of steps a) to c), the arenavirus is constantly forced to adapt to a new environment, i.e. to a new host animal. This permanent adaptation pressure favors the occurrence of mutations, which can produce or improve the tumor-regressive properties of the arenavirus.

During the culturing of the arenavirus according to step b), a replication of the arenavirus genome and a propagation of the arenavirus occur within the host animal.

The arenavirus is preferably cultured in the host animal for a period of 1 minute to 500 days, in particular 10 minutes to 100 days, preferably 1 hour to 30 days.

In a preferred embodiment, the arenavirus is isolated from urine, blood, the tumor, or organ lysates of the host animal.

In a further embodiment, the method further comprises the following steps:
d) infecting dendritic cells or tumor cells with the arenavirus isolated according to step c),
e) culturing the arenavirus in the infected dendritic cells or infected tumor cells and
f) isolating the cultured arenavirus or a subset of the cultured arenavirus from the infected dendritic cells or infected tumor cells.

The sequence of steps d) to f) may also be referred to as a (single) passage of the arenavirus in the dendritic cells or tumor cells.

The dendritic cells or tumor cells are infected according to step d) preferably by adding the arenavirus to the cells.

In a preferred embodiment, the sequence of steps d) to f) is repeated with new, in particular non-infected, dendritic cells, preferably of the same type, or with new, in particular non-infected, tumor cells, preferably of the same type (the same tumor type).

In a particularly preferred embodiment, the sequence of steps d) to f) is repeated many times. By way of preference, new, in particular non-infected, dendritic cells, preferably of the same type, or new, in particular non-infected, tumor cells, preferably of the same type, are used for each repetition. In other words, it is particularly preferred according to the invention if the arenavirus is additionally subjected to a serial passage in dendritic cells, preferably of the same type, or tumor cells, preferably of the same type.

The sequence of steps d) to f) is preferably repeated once to 1000 times, particularly 10 times to 100 times, preferably 30 times to 60 times, wherein new, in particular non-infected, dendritic cells, preferably of the same type, or new, in particular non-infected, tumor cells, preferably of the same type, are preferably used for each repetition.

The combination of a serial passage of the arenavirus in host animals, preferably of the same type, with a serial passage of the arenavirus in dendritic cells, preferably of the same type, or tumor cells, preferably of the same type, is suitable, because of the additionally increased adaptation pressure or mutation pressure in a particular manner, for producing arenaviruses with (improved) tumor-regressive properties.

The arenavirus is preferably cultured in the dendritic cells or tumor cells for a period of 1 minute to 500 days, in particular 10 minutes to 100 days, preferably 1 hour to 30 days.

Preferably, the dendritic cells or tumor cells are sorted according to specific properties, preferably by means of a cell sorting device, and subsequently cultured, prior to isolating according to step f). The sorted cells are preferably cultured over a period of 24 hours.

In a preferred embodiment, the method further comprises the following steps:
g) cloning the arenavirus isolated according to step f) and
h) sequencing the isolated arenavirus.

Alternatively, it can be provided according to the invention that the arenavirus isolated according to step c) is cloned and subsequently sequenced.

With regard to other features and advantages of the method, in particular the arenavirus, the tumor and also the dendritic cells and tumor cells, the present description is also fully incorporated by way of reference in order to avoid unnecessary repetitions.

Further features and advantages of the invention will emerge from the following description of preferred embodiments in the form of working examples, the associated figures and the claims. The embodiments described below are merely for the purpose of illustration and for the better understanding of the invention and are in no way to be understood as limiting.

### BRIEF DESCRIPTION OF THE DRAWINGS

FIG. 1 shows LCMV (WE strain) replication results in immortalized macrophages (“tumour”) and macrophages (“primary”) generated from primary bone marrow.

FIG. 2 shows the effect of LCMV (WE strain) administration on MOPC (mouse plasmacytoma cell) tumor growth in C57BL/6 mice.

FIG. 3 shows the effect of LCMV (WE strain) administration on MC38 (mouse colon adenocarcinoma cell) tumor growth in C57BL/6 mice.

FIG. 4 shows the effect of LCMV (WE strain) administration on spontaneous liver carcinoma tumor growth in LoxP-TAg mice.

FIG. 5 shows the effect of LCMV (WE strain) on interferon-γ secretion in MOPC tumor-bearing C57BL/6 mice.

FIG. 6 shows the effect of LCMV (WE strain) administration on MOPC tumor growth in Map3k14^{aly/aly} mice compared to corresponding wild type mice.

FIG. 7 shows the effect of LCMV (WE strain) administration on MOPC tumor growth in Irf3×Ir7^{-/-} mice compared to corresponding wild type mice.

FIG. 8A shows effect of LCMV (WE strain) administration on tumor microvessel density (MDV) and vessel-vessel spacing in MOPC tumor-bearing wild type mice.

FIG. 8B shows effect of LCMV (WE strain) administration on tumor hypoxic regions in MOPC tumor-bearing wild type mice.

FIG. 9 shows the effect of LCMV (WE strain) administration (ipsilateral, contralateral, and intravenous) on MOPC tumor growth in C57BL/6 mice.

FIG. 10 shows effect of LCMV (WE strain) administration on survival in A431 (squamous carcinoma) tumor-bearing NOD.SCID mice.

FIG. 11 shows effect of LCMV (WE strain) administration on tumor growth in A431 (colon adenocarcinoma) tumor-bearing NOD.SCID mice.

FIG. 12 shows effect of Candid #1 administration on tumor growth in HeLa cell-bearing NOD.SCID mice.

FIG. 13 shows effect of Candid #1 administration on tumor growth in HepG2 (hepatocellular carcinoma) tumor-bearing NOD.SCID mice.

FIG. 14 shows LCMV (MOI 1) replication results in primary hepatocytes, colon epithelial cells, and melanocytes) and in tumour cells from the same tissue source.

FIG. 15A shows the effect of LCMV administration on metastatic MOPC tumor growth in C57BL/6 mice.

FIG. 15B shows the effect of LCMV administration on survival in MOPC metastatic tumor-bearing C57BL/6 mice.

FIG. 16 shows the effect of LCMV administration on B16F10 (melanoma) tumor growth in C57BL/6 mice.

FIG. 17 shows the effect of LCMV administration on spontaneous melanoma tumor growth in MT/ret mice.

FIG. 18A shows the effect of LCMV administration on Sw872 (fibrosarcoma) tumor growth in NOD.SCID mice.

FIG. 18B shows the effect of LCMV administration on survival in Sw872 (fibrosarcoma) tumor-bearing NOD.SCID mice.

FIG. 19A shows the effect of LCMV administration on FaDu (pharyngeal carcinoma) tumor growth in NOD.SCID mice.

FIG. 19B shows the effect of LCMV administration on survival in FaDu (pharyngeal carcinoma) tumor-bearing NOD.SCID mice.

FIG. 20 shows the effect of LCMV on T-cell expression of PD-1, IL2 receptor, and IL7 receptor in B16F10 tumor-bearing mice.

FIG. 21A shows the effect of LCMV administration on EL4 (lymphoma) tumor growth in C57BL/6 mice.

FIG. 21B shows the effect of LCMV administration on survival in EL4 (lymphoma) tumor-bearing C57BL/6 mice.

FIG. 22 shows the effect of LCMV administration on survival in MOPC tumor-bearing C57BL/6 and Pdcd1^{-/-}mice.

### EXPERIMENTAL SECTION

### 1. Methods and Materials

### 1.1 Mice

Unless mentioned otherwise, the mice used were from a C57BL/6 background. Map3k14^{aly/aly} mice lack NF-kB signals and are therefore highly immunosuppressed. Irf3×Ir7^{-/-} mice cannot produce any interferon. NOD.SCID mice have no adaptive immune system. Therefore, it is possible to grow human tumors in these mice. LoxP-Tag mice spontaneously develop liver tumors.

### 1.2 Cell Lines and Reagents

MOPC cells are murine oropharynx carcinoma cells. Mc38 are murine colon carcinoma cells. Raw cells are immortalized macrophages. A431 are human lung carcinoma cells; Sw40 are human colon carcinoma cells, Hela are human cervical carcinoma cells. Primary macrophages were cultured from bone marrow precursor cells by means of M-CSF. Cells were maintained in Dulbecco's modified Eagle's medium with 10% fetal bovine serum (Sigma-Aldrich), 2 mmol/l L-glutamine and 100 U/ml penicillin. All cells were cultured in 5% CO2.

### 1.3 Viruses

The LCMV strain WE was obtained from the laboratory of Prof. Zinkernagel (Experimental Immunology, Zurich, Switzerland) and was propagated in L929 cells. Candid #1 was obtained from Professor Paula Cannon (University of Southern California).

### 1.4 Tumor Growth and Treatments

Approximately 5×10⁵ tumor cells (in 100 microL) were injected subcutaneously into the right flank of 6 to 12 week old mice. The longest tumor diameter was measured by. Mice were treated by peritumoral injections of 2×10⁴ PFU LCMV-WE or Candid #1 (in 100-200 microL).

### 1.5 Morphometric Analysis of Tumor Vessels

Morphometric analyses were performed with successive frozen sections, in which the endothelial cell marker CD31 was stained. Quantification of the microvessel density (MVD) was calculated using the mean of three tumor sections. MVD was calculated as the number of vessels per tumor area.

### 1.6 Detection of Hypoxia

Hypoxic tumor regions were detected by the formation of pimonidazole adducts after injection of pimonidazole into tumor-transplanted animals for 30 min. The tumor sections were stained using the Hypoxyprobe-1 Plus kit according to the manufacturer's instructions (Pharmacia Natur International, Inc.).

### 1.7 IFN-α ELISA

Serum IFN-α levels were determined by ELISA according to the manufacturer's data (Research Diagnostics RDI, Flanders, NJ).

### 1.8 Statistical Analysis

The mean values were compared using an unpaired two-sided student t-test. The data are shown as mean+SEM. The level of statistical significance was set at p<0.05.

### 2. Investigations

2.1 Immortalized Macrophages (Tumour Cells) and Macrophages (Primary) generated from primary bone marrow were infected with LCMV (WE strain). Replication was measured after 24 hours (n=3).

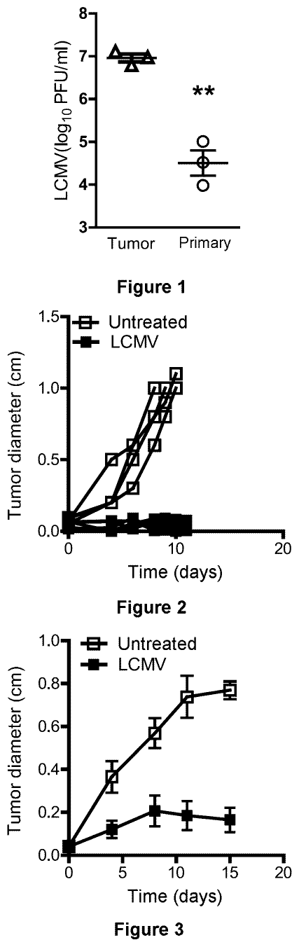

It could be shown that LCMV (WE strain) replicates both in immortalized and healthy cells. The results obtained are shown graphically in FIG. 1.
FIG. 1 has the following legend:
Ordinate: LCMV (log₁₀ PFU/ml)
Abscissa: Tumor cells/healthy macrophages (primary)

2.2 WT C57BL/6 mice were treated with 5×10⁵ MOPC cells (day 3). One group of mice was additionally treated peritumorally with 2×10⁴ PFU LCMV (WE strain) (day 0). Tumor growth was observed.

It could be shown that the treatment with LCMV caused almost complete tumor regression. The results obtained are shown graphically in FIG. 2.
FIG. 2 has the following legend:
Ordinate: Tumor diameter (cm)
Abscissa: Time (days)

2.3 WT C57BL/6 mice were treated with 5×10⁵ MC38 cells (day 3). One group of mice was additionally treated peritumorally with 2×10⁴ PFU LCMV (WE strain) (day 0). Tumor growth was observed.

It could be shown that the treatment with LCMV caused a significant tumor regression. The results obtained are shown graphically in FIG. 3.
FIG. 3 has the following legend:
Ordinate: Tumor diameter (cm)
Abscissa: Time (days)

2.4 About nine month old LoxP-TAg mice with spontaneously developed liver carcinomas were infected intravenously with 2×10⁶ PFU LCMV or left untreated. The tumor nodes (diameters>=3 mm) were quantified macroscopically on day 6 (n=3) and day 20 (n=4-5).

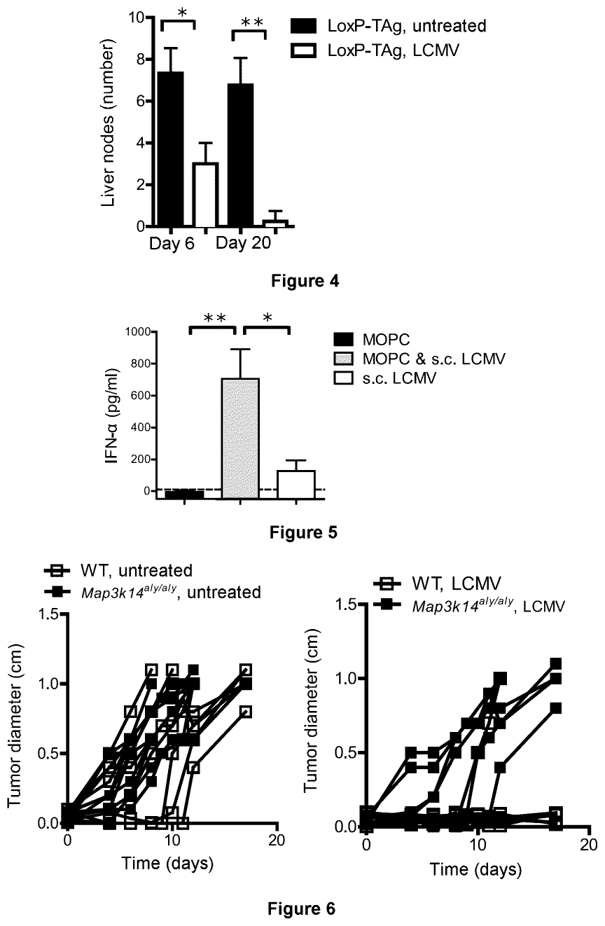

It could be shown that the treatment with LCMV significantly reduced the number of carcinomatous liver nodes. The results obtained are shown graphically in FIG. 4.
FIG. 4 has the following legend:
Ordinate: Liver nodes (number)
Abscissa: Time

2.5 WT C57BL/6 mice (n=4/group) were injected subcutaneously with 5×10⁵ MOPC cells (day −3) or LCMV (WE strain) 2×10⁴ PFU (day 0) or both 5×10⁵ MOPC cells (day −3) and 2 33 10⁴ PFU LCMV (day 0). Serum samples were collected on day 3 and an IFN-α-ELISA was performed.

It could be shown that the LCMV caused a drastically increased secretion of interferon-γ in experimental animals which were simultaneously administered carcinoma cells. The results obtained are shown graphically in FIG. 5.
FIG. 5 has the following legend:
Ordinate: IFN-α (pg/ml)

2.6 Map3k14^{aly/aly} mice and WT mice were treated with 5×10⁵ MOPC cells (day 3). One group of mice was additionally treated peritumorally with 2×10⁴ PFU LCMV (WE strain) (day 0). Tumor growth was observed.

It could be shown that the treatment with LCMV caused tumor regression. The results obtained are shown graphically in FIG. 6.
FIG. 6 has the following legend:
Ordinate: Tumor diameter (cm)
Abscissa: Time (days)

2.7 Irf3×Ir7^{-/-} mice and WT mice were treated with 5×10⁵ MOPC cells (day 3). One group of mice was additionally treated peritumorally with 2×10⁴ PFU LCMV (WE strain) (day 0). Tumor growth was observed.

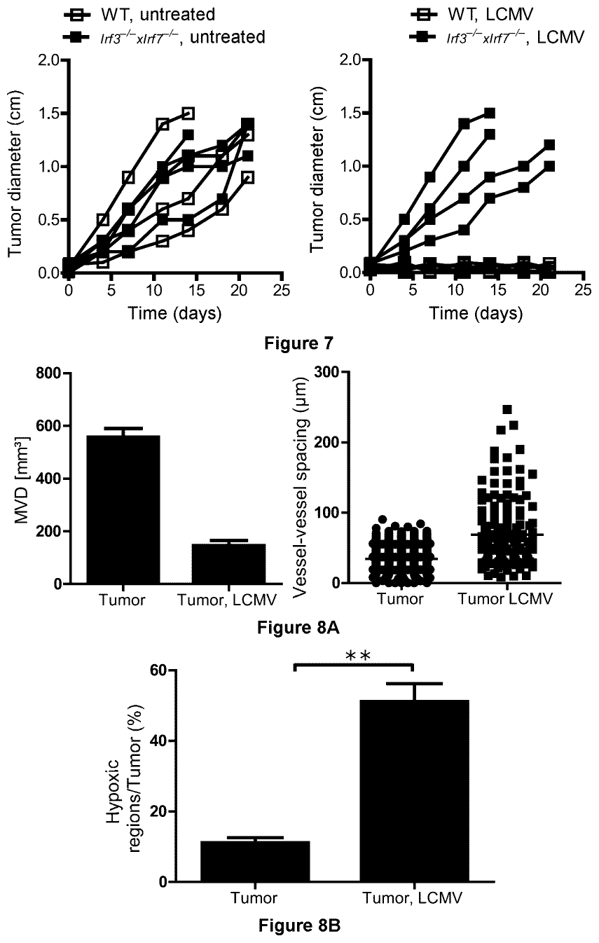

It could be shown that the treatment with LCMV caused tumor regression. The results obtained are shown graphically in FIG. 7.
FIG. 7 has the following legend:
Ordinate: Tumor diameter (cm)
Abscissa: Time (days)

2.8 WT mice were treated with 5×10⁵ MOPC cells (day 3). One group of mice was additionally treated peritumorally with 2×10⁴ PFU LCMV (WE strain) (day 0). On day 9 after the tumor graft, the tumors were analyzed histologically with CD31 staining. The microvessel density (MDV) and the vessel-vessel spacing were quantified.

It could be shown that the treatment with LCMV caused a decrease in tumor vessel density. The results obtained are shown graphically in FIG. 8A.
FIG. 8A has the following legends:
Left side: Right side:
Ordinate: MVD [mm³]
Ordinate: Vessel-vessel spacing (μm)
Abscissa: Tumor/Tumor LCMV Abscissa: Tumor/Tumor LCMV

2.9 WT mice were treated with 5×10⁵ MOPC cells (day 3). One group of mice was additionally treated peritumorally with 2×10⁴ PFU LCMV (WE strain) (day 0). On day 9, the animals were injected with pimonidazole, and the tumors were then analyzed histologically for hypoxic regions.

It could be shown that the treatment with LCMV caused oxygen deficiency in the carcinoma tissue. The results obtained are shown graphically in FIG. 8B.
FIG. 8B has the following legend:
Ordinate: Hypoxic regions/tumor (%)
Abscissa: Tumor/Tumor LCMV

2.10 WT C57BL/6 mice were injected subcutaneously with 5×10⁵ MOPC cells in the right flank (day 3). On day 0, a group of animals were treated with 2×10⁴ PFU LCMV (WE strain) in the right flank (ipsilateral), left flank (contralateral) or intravenously. Tumor growth was observed.

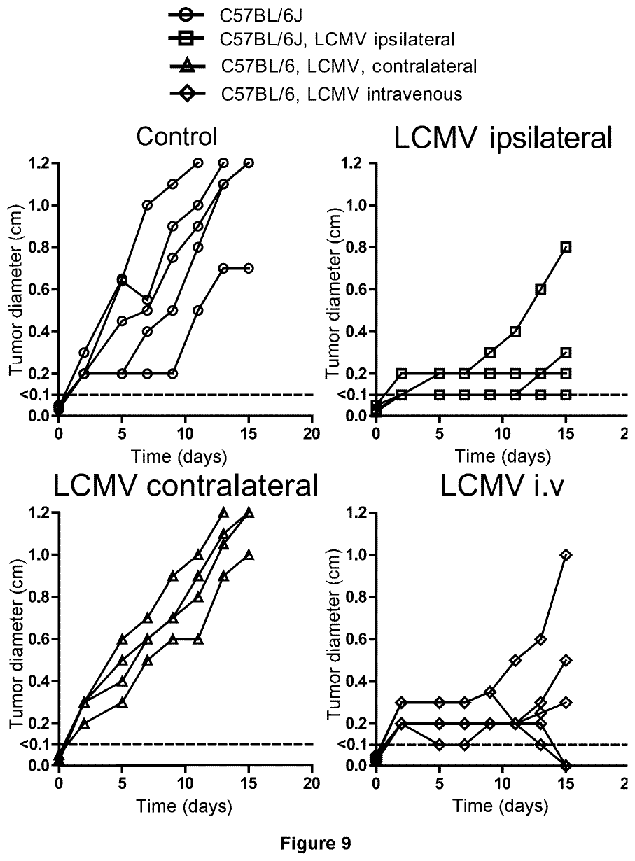

It could be shown that the treatment with LCMV caused tumor regression even with systemic administration. The results obtained are shown graphically in FIG. 9.
FIG. 9 has the following legend:
Ordinate: Tumor diameter (cm)
Abscissa: Time (days)

2.11 NOD.SCID mice were injected subcutaneously with 5×10⁵ A431 cells (day −3) and then either left untreated or treated with 2×10⁴ PFU LCMV (WE strain). The tumor size (longest diameter) was measured on the specified day. The mice were sacrificed when the tumor size reached 12 mm.

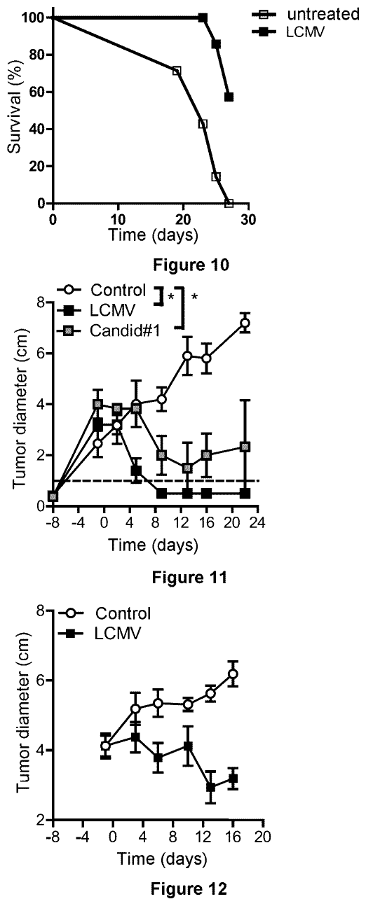

It could be shown that the treatment with LCMV increased the survival rate in the experimental animals. The results obtained are shown graphically in FIG. 10.
FIG. 10 has the following legend:
Ordinate: Survival (%)
Abscissa: Time (days)

2.12 NOD.SCID mice were treated with 5×10⁵ Sw40 cells (day 0). A group of mice was additionally treated peritumorally with 2×10⁴ PFU LCMV (WE strain) or 2×10⁴ PFU Candid #1 (day 0). Tumor growth was observed.

It could be shown that the treatment with LCMV and Candid #1 caused tumor regression. The results obtained are shown graphically in FIG. 11.
FIG. 11 has the following legend:
Ordinate: Tumor diameter (cm)
Abscissa: Time (days)

2.13 NOD.SCID mice were treated with 5×10⁵ Hela cells (day 0). A group of mice was additionally treated peritumorally with 2×10⁴ PFU LCMV (WE strain) (day 3). Tumor growth was observed.

It could be shown that the treatment with LCMV caused tumor regression. The results obtained are shown graphically in FIG. 12.
FIG. 12 has the following legend:
Ordinate: Tumor diameter (cm)
Abscissa: Time (days)

2.14 NOD.SCID mice were treated with 5×10⁵ HepG2 cells (day 10) and then additionally treated peritumorally with or without 2×10⁴ PFU Candid #1 (day 0). Tumor growth was observed.

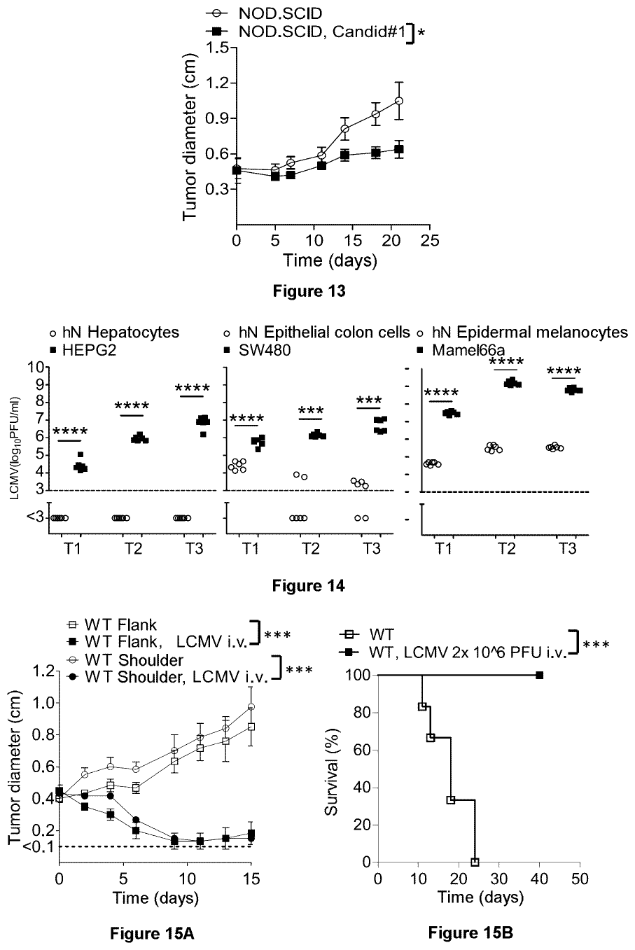

It could be shown that the treatment with Candid #1 caused tumor regression with this tumor type. The results obtained are shown graphically in FIG. 13.
FIG. 13 has the following legend:
Ordinate: Tumor diameter (cm)
Abscissa: Time (days)

2.15 Primary human cells (hepatocytes, colon epithelial cells, melanocytes) and tumour cells from the same tissue source were infected with LCMV (MOI 1). The amount of virus was measured in the supernatant after 1, 2 and 3 days.

In this experiment it was shown that arenaviruses are replicated in tumor cells in comparison to healthy tissue.
FIG. 14 has the following legend:
Ordinate: Infectious virus in cell culture supernatant (logarithmic plaque forming units)
Abscissa: Time (days)

2.16 Tumor diameter (A) and survival (B) of C57BL/6 mice bearing a metastasis in the shoulder and a metastasis in the flank (MOPC cells), which were left untreated or had been treated intravenously with 2×10⁶ PFU LCMV.

It could be shown in this experiment that intravenous therapy of LCMV acts very efficiently on two local metastases and thus prolongs survival.
FIG. 15A has the following legend:
Ordinate: Tumor diameter of both metastases (cm)
Abscissa: Time (days)
   FIG. 15B has the following legend:
Ordinate: Survival in percent
Abscissa: Time (days)

2.17 Tumor diameter of C57BL/6 mice bearing a melanoma (B16F10 cells) which were left untreated or were treated intratumorally with 2×10⁴ PFU LCMV.

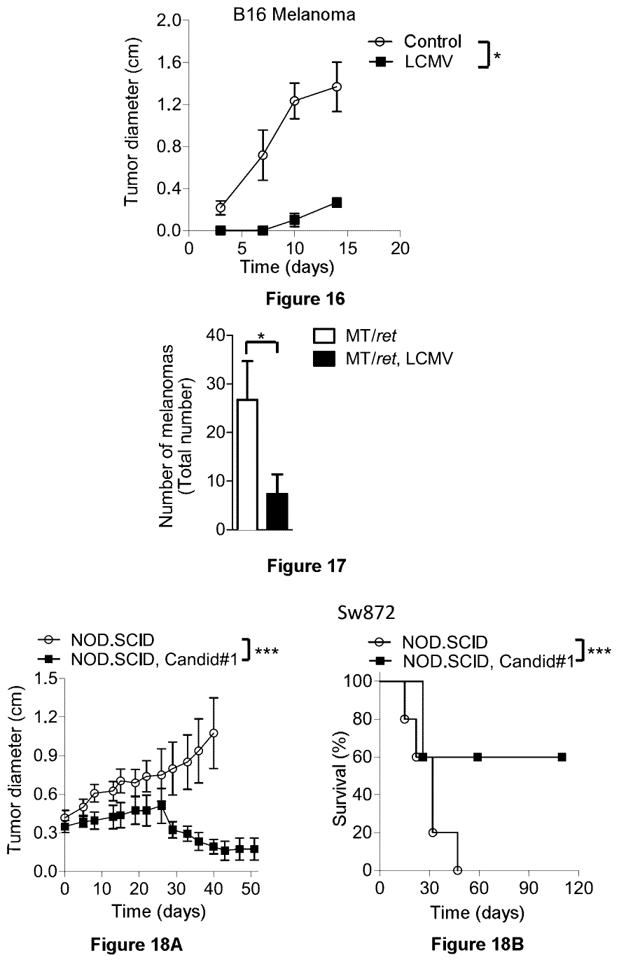

It could be shown in this experiment that local therapy with LCMV is very efficient in melanoma.
FIG. 16 has the following legend:
Ordinate: Tumor diameter of the melanoma (cm)
Abscissa: Time (days)

2.18 Number of melanomas of MT/ret mice (develop endogenous melanomas), which were left untreated or were treated intravenously with 2×10⁶ PFU LCMV.

It could be shown in this experiment that systemic therapy with LCMV is very efficient in melanoma.
FIG. 17 has the following legend:
Ordinate: Number of melanomas

2.19 Tumor diameter (A) and survival (B) of NOD.SCID mice bearing a human fibrosarcoma (Sw872 cells), which were left untreated or were treated intratumorally with 2×10⁶ PFU Candid #1.

It could be shown in this experiment that Candid #1 acts very efficiently also in the case of fibrosarcoma and thus prolongs survival.
FIG. 18A has the following legend:
Ordinate: Tumor diameter (cm)
Abscissa: Time (days)
   FIG. 18B has the following legend:
Ordinate: Survival in percent
Abscissa: Time (days)

2.20 Tumor diameter (A) and survival (B) of NOD.SCID mice bearing a human pharyngeal carcinoma (FaDu cells), which were left untreated or were treated intratumorally with 2×10⁶ PFU LCMV.

It could be shown in this experiment that LCMV acts very efficiently also in the case of pharyngeal carcinoma and thus prolongs survival.
FIG. 19A has the following legend:
Ordinate: Tumor diameter (cm)
Abscissa: Time (days)
   FIG. 19B has the following legend:
Ordinate: Survival in percent
Abscissa: Time (days)

2.21 Expression of receptors on tumor-specific T cells (PD-1, IL2 receptor, IL7 receptor), which influence the function of T cells. Tumor-specific T cells are derived from the blood of mice with B16F10 tumors, which were additionally treated intratumorally with or without LCMV.

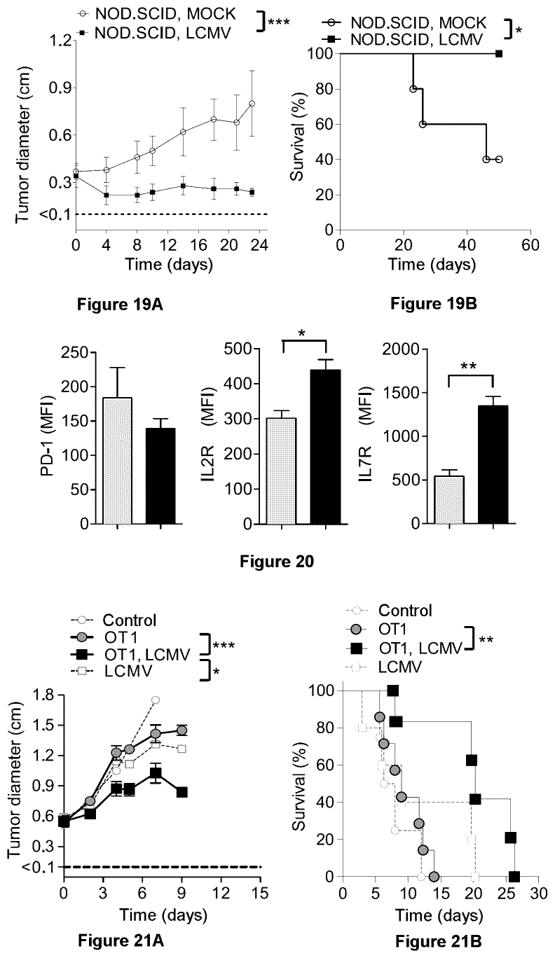

It could be shown in this experiment that LCMV positively influences the tumor-specific T cells.
FIG. 20 has the following legend:
Ordinate: Potency of the expression of the different receptors (mean fluorescence intensity)

2.22 Tumor diameter (A) and survival (B) of C57BL/6 mice bearing a murine subcutaneous lymphoma (EL4 cells) which were treated with or without tumor-specific T cells (OT1 cells) and additionally intratumorally with or without LCMV (2×10⁶ PFU).

It could be shown in this experiment that LCMV acts synergistically with T cell therapy.
FIG. 21A has the following legend:
Ordinate: Tumor diameter (cm)
Abscissa: Time (days)
   FIG. 21B has the following legend:
Ordinate: Survival in percent
Abscissa: Time (days)

2.23 Survival of C57BL/6 mice and PD-1 deficient mice (Pdcd1^{-/-} mice) bearing a murine pharyngeal carcinoma (MOPC cells) and which were treated intratumorally with LCMV (2×10⁴ PFU).

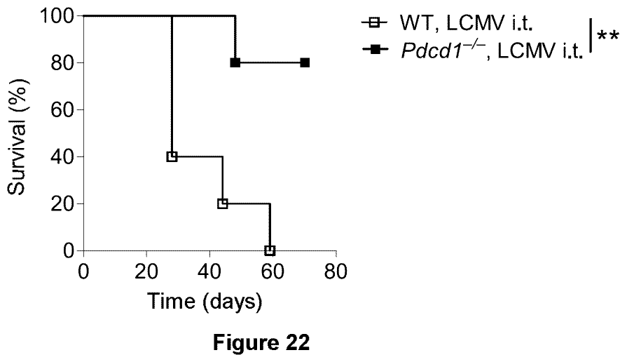

It could be shown in this experiment that LCMV acts synergistically with a PD-1 blockade.
FIG. 22 has the following legend:
Ordinate: Survival in percent
Abscissa: Time (days)

The nucleic acid sequences SEQ ID No. 1 to SEQ ID No. 6 mentioned in the general description correspond to the nucleic acid sequences disclosed in the following sequence listing.

1. A method of treating and/or preventing a tumor comprising administering to a subject in need thereof an arenavirus capable of effecting tumor regression by activating innate (congenital) immune cells, adaptive immune cells, or both innate (congenital) immune cells and adaptive immune cells.

2. The method of claim 1, wherein the method comprises activating innate (congenital) or adaptive immune cells and thereby effecting tumor regression.

3. The method of claim 1, wherein the method comprises effecting increased secretion of anti-tumoral cytokines by immune cells by activating said immune cells.

4. The method of claim 4, wherein the anti-tumoral cytokine is interferon-α, interferon-β or interferon-γ.

5. The method of claim 1, wherein the arenavirus is a wild-type arenavirus.

6. The method of claim 1, wherein the arenavirus is a natural mutant of a wild-type arenavirus.

7. The method of claim 1, wherein the arenavirus has been obtained starting from its wild-type form by serial passage in a host animal and/or a host cell.

8. The method of claim 1, wherein the tumor is selected from the group consisting of carcinoma, melanoma, blastoma, lymphoma, and sarcoma.

9. The method of claim 1, wherein the arenavirus is an Old World arenavirus.

10. The method of claim 1, wherein the arenavirus is lymphocytic choriomeningitis virus.

11. The method of claim 10, wherein the arenavirus comprises a nucleic acid comprising an S-ribonucleic acid sequence according to SEQ ID No. 5 or ambisense sequence thereof, or a nucleic acid comprising an L-ribonucleic acid sequence according to SEQ ID No. 6 or ambisense sequence thereof.

12. The method of claim 10, wherein the lymphocytic choriomeningitis virus is a lymphocytic choriomeningitis virus strain WE or a natural mutant thereof.

13. The method of claim 1, wherein the arenavirus is a New World arenavirus.

14. The method of claim 1, wherein the arenavirus is a Junin virus.

15. The method of claim 14, wherein the arenavirus comprises a nucleic acid comprising an S-ribonucleic acid sequence according to SEQ ID No. 1 or 3 or ambisense sequence thereof, or a nucleic acid comprising an L-ribonucleic acid sequence according to SEQ ID No. 2 or 4 or ambisense sequence thereof.

16. The method of claim 14, wherein the Junin virus is strain Candid #1.

17. The method of claim 1, wherein the subject is a human.

18. A pharmaceutical composition comprising an arenavirus of claim 1 and, optionally a pharmaceutically acceptable carrier.

19. A method for generating an arenavirus with tumor-regressive or improved tumor-regressive properties, in particular an arenavirus according to claim 1, comprising:
a) infecting dendritic cells or tumor cells with an arenavirus,
b) culturing the arenavirus in the infected dendritic cells or infected tumor cells and
c) isolating the cultured arenavirus or a subset of the cultured arenavirus from the infected dendritic cells or infected tumor cells.

20. The method of claim 19, wherein the sequence of steps a) to c) is repeated once to 1000 times, particularly 10 times to 100 times, preferably 30 times to 60 times, wherein new tumor cells, preferably of the same type, are used for each repetition.
